(19) United States
(12) Patent Application Publication
(10) Pub. No.: US 2026/0331217 A1
(43) Pub. Date: Oct. 22, 2026

(54) METHODS AND SYSTEMS FOR DETECTING HOWELL-JOLLY BODIES

(71) Applicant: Foundation Medicine, Inc., Boston, MA (US)

(72) Inventors: Dean PAVLICK, Cambridge, MA (US); Garrett M. FRAMPTON, Somerville, MA (US); Jonathan Keith KILLIAN, Cambridge, MA (US); Bernard FENDLER, Auburndale, MA (US)

(73) Assignee: Foundation Medicine, Inc., Boston, MA (US)

(21) Appl. No.: 19/464,161

(22) Filed: Jan. 29, 2026

Related U.S. Application Data
(60) Provisional application No. 63/752,155, filed on Jan. 31, 2025.
(60) Provisional application No. 63/962,174, filed on Jan. 16, 2026.

(51) Int. Cl. G16B 20/10; G16B 5/20; G16B 45/00; G16H 20/10; G16H 50/20

(52) U.S. Cl. CPC G16B 20/10; G16B 5/20; G16B 45/00; G16H 20/10; G16H 50/20

(57) ABSTRACT

Methods for detecting Howell-Jolly bodies from sequencing data are described. The methods may comprise, for example, receiving, using one or more processors, genomic data from a sample from a subject; determining, using the one or more processors, a copy number at one or more genomic loci based on the genomic data; determining, using the one or more processors, a distance for each of the one or more genomic loci from a centromere of a chromosome; and determining a correlation, using the one or more processors, wherein the correlation comprises a change in the determined copy number as a function of the determined distances. The methods may further comprise detecting, using the one or more processors, a presence or an absence of a disease marker in the sample from the subject based on the correlation, wherein the disease marker comprises a Howell-Jolly body.

### CROSS-REFERENCE TO RELATED APPLICATIONS

This application claims priority to and benefit of U.S. Provisional Application No. 63/752,155, filed Jan. 31, 2025, and U.S. Provisional Application No. 63/962,174, filed Jan. 16, 2026, the contents of each of which are incorporated herein by reference in their entireties.

### FIELD

The present disclosure relates generally to methods and systems for detecting Howell-Jolly bodies (HJBs) from genomic data, and more specifically to methods and systems for determining correlations between copy number variants and genomic distances to detect HJBs as markers of splenic dysfunction.

### BACKGROUND

Howell-Jolly bodies (HJBs) are nuclear remnants in erythrocytes (red blood cells). In healthy subjects, erythrocytes are produced in the bone marrow and released into the peripheral blood. As part of normal erythrocyte physiology, HJBs are generated and shed into the peripheral blood. However, in healthy subjects with normal splenic function, HJBs are filtered out by the spleen, and thus are typically not detectable in routine blood analyses. In subjects with asplenia (absent splenic function) or hyposplenia (reduced splenic function), HJBs can persist in peripheral blood. Asplenia or hyposplenia can be caused by splenectomy (anatomical removal of the spleen), congenital absence of the spleen, or disease impacting splenic function and/or blood cell physiology (including, but not limited to, heme diseases, immunological diseases, and hematological cancers).

The presence of HJBs can be used as a marker to identify absent or reduced splenic function and to monitor, diagnose, or treat diseases involving splenic dysfunction. Currently, HJB detection is limited to microscopic evaluation of peripheral blood smears by pathologists. HJBs appear as dark inclusions when erythrocytes are visualized microscopically. However, microscopic evaluation is low throughput, prone to inaccuracies, and requires pathologist expertise. Flow cytometry and automated hematology analyzers may also be used to detect the presence of abnormal erythrocytes harboring nucleic acid, some of which could be HJBs. However, these approaches are similarly limited in throughput. There is an ongoing need to develop improved approaches to detecting the presence of HJBs in peripheral blood for use in diagnosing, monitoring, and treating disease.

### BRIEF SUMMARY OF THE INVENTION

Disclosed herein are methods and systems for detecting HJBs from genomic data and determining correlations between copy number variants and genomic distances to detect HJBs based on genomic data. HJBs are nuclear remnants that are found in erythrocytes and typically removed from peripheral blood by the spleen in healthy subjects. In subjects with certain pathologies, HJBs may persist in the peripheral blood. Persistent HJBs are often caused by asplenia or hyposplenia, as normal splenic function is required for filtration of erythrocytes and the removal of abnormalities such as HJBs. HJB detection is important for understanding diseases that result in splenic dysfunction, and can inform diagnosis, monitoring, and treatment strategies.

While it is possible to detect HJBs using microscopic visualization of peripheral blood smears, this approach is low throughput, prone to inaccuracies, and requires pathologist evaluation. The present invention offers a higher throughput approach with increased accuracy and sensitivity. The disclosed methods and systems can be used to detect HJBs based on a characteristic genomic signature. This approach allows the use of highly sensitive genomic technologies for sequencing of samples from subjects and subsequent detection of HJBs. Liquid biopsy samples often contain peripheral blood and can be sequenced as described herein to detect HJBs as a marker of splenic dysfunction.

Using the methods and systems disclosed herein, HJBs can be detected and quantified at levels far below what may be achievable with routine histological detection in peripheral blood smears. Quantifying the characteristic genomic signature of HJBs can be used to assess and monitor splenic function in subjects where asplenia or hyposplenia are relevant to diagnosis and treatment. Monitoring for HJBs is not currently a part of routine cancer diagnosis and treatment. HJB detection as described herein could be used to signal the presence of cancer or other disease states impeding splenic function.

In some aspects, disclosed herein is a method comprising: providing a plurality of nucleic acid molecules obtained from a sample from a subject; ligating one or more adapters onto one or more nucleic acid molecules from the plurality of nucleic acid molecules; amplifying the one or more ligated nucleic acid molecules from the plurality of nucleic acid molecules; capturing amplified nucleic acid molecules from the amplified nucleic acid molecules; sequencing, by a sequencer, the captured nucleic acid molecules to obtain a plurality of sequence reads that represent the captured nucleic acid molecules; receiving, using one or more processors, sequence read data for the plurality of sequence reads; determining, using the one or more processors, a copy number at one or more genomic loci based on the sequence read data; determining, using the one or more processors, a distance for each of the one or more genomic loci from a centromere of a chromosome; and determining a correlation, using the one or more processors, wherein the correlation comprises a change in the determined copy number as a function of the determined distances.

In some aspects, the method further comprises detecting, using the one or more processors, a presence or an absence of a disease marker in the sample based on the correlation. In some aspects, the disease marker comprises a Howell-Jolly Body (HJB). In some aspects, the method further comprises diagnosing a disease in the subject based on the detecting of the presence or the absence of the disease marker in the sample from the subject. In some aspects, the method further comprises treating the subject for the disease based on the detecting of the presence or the absence of the disease marker in the sample from the subject. In some aspects, the disease comprises asplenia, hyposplenia, or leukopenia. In some aspects, the presence or the absence of HJBs informs a decision regarding treatment of the subject with an immunotherapy. In some aspects, the presence of HJBs informs a contraindication for an immunotherapy.

In some aspects, the correlation is a non-linear correlation. In some aspects, the non-linear correlation is an exponential correlation. In some aspects, the correlation comprises an exponential increase in the copy number as a function of the distance for each of the one or more genomic loci from the centromere of the chromosome. In some aspects, the correlation is a linear correlation. In some aspects, the correlation comprises a linear increase in the copy number as a function of the distance for each of the one or more genomic loci from the centromere of the chromosome.

In some aspects, the subject is suspected of having or is determined to have cancer. In some aspects, the cancer is a B cell cancer (multiple myeloma), a melanoma, breast cancer, lung cancer, bronchus cancer, colorectal cancer, prostate cancer, pancreatic cancer, stomach cancer, ovarian cancer, urinary bladder cancer, brain cancer, central nervous system cancer, peripheral nervous system cancer, esophageal cancer, cervical cancer, uterine cancer, endometrial cancer, cancer of an oral cavity, cancer of a pharynx, liver cancer, kidney cancer, testicular cancer, biliary tract cancer, small bowel cancer, appendix cancer, salivary gland cancer, thyroid gland cancer, adrenal gland cancer, osteosarcoma, chondrosarcoma, a cancer of hematological tissue, an adenocarcinoma, an inflammatory myofibroblastic tumor, a gastrointestinal stromal tumor (GIST), colon cancer, multiple myeloma (MM), myelodysplastic syndrome (MDS), myeloproliferative disorder (MPD), acute lymphocytic leukemia (ALL), acute myelocytic leukemia (AML), chronic myelocytic leukemia (CML), chronic lymphocytic leukemia (CLL), polycythemia Vera, Hodgkin lymphoma, non-Hodgkin lymphoma (NHL), soft-tissue sarcoma, fibrosarcoma, myxosarcoma, liposarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, squamous cell carcinoma, basal cell carcinoma, adenocarcinoma, sweat gland carcinoma, sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, seminoma, embryonal carcinoma, Wilms' tumor, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, neuroblastoma, retinoblastoma, follicular lymphoma, diffuse large B-cell lymphoma, mantle cell lymphoma, hepatocellular carcinoma, thyroid cancer, gastric cancer, head and neck cancer, small cell cancer, essential thrombocythemia, agnogenic myeloid metaplasia, hypereosinophilic syndrome, systemic mastocytosis, familiar hypereosinophilia, chronic eosinophilic leukemia, neuroendocrine cancers, or a carcinoid tumor.

In some aspects, the cancer comprises acute lymphoblastic leukemia (Philadelphia chromosome positive), acute lymphoblastic leukemia (precursor B-cell), acute myeloid leukemia (FLT3+), acute myeloid leukemia (with an IDH2 mutation), anaplastic large cell lymphoma, basal cell carcinoma, B-cell chronic lymphocytic leukemia, bladder cancer, breast cancer (HER2 overexpressed/amplified), breast cancer (HER2+), breast cancer (HR+, HER2−), cervical cancer, cholangiocarcinoma, chronic lymphocytic leukemia, chronic lymphocytic leukemia (with 17p deletion), chronic myelogenous leukemia, chronic myelogenous leukemia (Philadelphia chromosome positive), classical Hodgkin lymphoma, colorectal cancer, colorectal cancer (dMMR/MSI-H), colorectal cancer (KRAS wild type), cryopyrin-associated periodic syndrome, a cutaneous T-cell lymphoma, dermatofibrosarcoma protuberans, a diffuse large B-cell lymphoma, fallopian tube cancer, a follicular B-cell non-Hodgkin lymphoma, a follicular lymphoma, gastric cancer, gastric cancer (HER2+), gastroesophageal junction (GEJ) adenocarcinoma, a gastrointestinal stromal tumor, a gastrointestinal stromal tumor (KIT+), a giant cell tumor of the bone, a glioblastoma, granulomatosis with polyangiitis, a head and neck squamous cell carcinoma, a hepatocellular carcinoma, Hodgkin lymphoma, juvenile idiopathic arthritis, lupus erythematosus, a mantle cell lymphoma, medullary thyroid cancer, melanoma, a melanoma with a BRAF V600 mutation, a melanoma with a BRAF V600E or V600K mutation, Merkel cell carcinoma, multicentric Castleman's disease, multiple hematologic malignancies including Philadelphia chromosome-positive ALL and CML, multiple myeloma, myelofibrosis, a non-Hodgkin's lymphoma, a nonresectable subependymal giant cell astrocytoma associated with tuberous sclerosis, a non-small cell lung cancer, a non-small cell lung cancer (ALK+), a non-small cell lung cancer (PD-L1+), a non-small cell lung cancer (with ALK fusion or ROS1 gene alteration), a non-small cell lung cancer (with BRAF V600E mutation), a non-small cell lung cancer (with an EGFR exon 19 deletion or exon 21 substitution (L858R) mutations), a non-small cell lung cancer (with an EGFR T790M mutation), ovarian cancer, ovarian cancer (with a BRCA mutation), pancreatic cancer, a pancreatic, gastrointestinal, or lung origin neuroendocrine tumor, a pediatric neuroblastoma, a peripheral T-cell lymphoma, peritoneal cancer, prostate cancer, a renal cell carcinoma, rheumatoid arthritis, a small lymphocytic lymphoma, a soft tissue sarcoma, a solid tumor (MSI-H/dMMR), a squamous cell cancer of the head and neck, a squamous non-small cell lung cancer, thyroid cancer, a thyroid carcinoma, urothelial cancer, a urothelial carcinoma, or Waldenstrom's macroglobulinemia.

In some aspects, the method further comprises treating the subject with an anti-cancer therapy. In some aspects, the anti-cancer therapy comprises a targeted anti-cancer therapy. In some aspects, the targeted anti-cancer therapy comprises abemaciclib (Verzenio), abiraterone acetate (Zytiga), acalabrutinib (Calquence), ado-trastuzumab emtansine (Kadcyla), afatinib dimaleate (Gilotrif), alectinib (Alecensa), alemtuzumab (Campath), alitretinoin (Panretin), alpelisib (Piqray), amivantamab-vmjw (Rybrevant), anastrozole (Arimidex), apalutamide (Erleada), asciminib hydrochloride (Scemblix), atezolizumab (Tecentriq), avapritinib (Ayvakit), avelumab (Bavencio), axicabtagene ciloleucel (Yescarta), axitinib (Inlyta), belantamab mafodotin-blmf (Blenrep), belimumab (Benlysta), belinostat (Beleodaq), belzutifan (Welireg), bevacizumab (Avastin), bexarotene (Targretin), binimetinib (Mektovi), blinatumomab (Blincyto), bortezomib (Velcade), bosutinib (Bosulif), brentuximab vedotin (Adcetris), brexucabtagene autoleucel (Tecartus), brigatinib (Alunbrig), cabazitaxel (Jevtana), cabozantinib (Cabometyx), cabozantinib (Cabometyx, Cometriq), canakinumab (Ilaris), capmatinib hydrochloride (Tabrecta), carfilzomib (Kyprolis), cemiplimab-rwlc (Libtayo), ceritinib (LDK378/Zykadia), cetuximab (Erbitux), cobimetinib (Cotellic), crizotinib (Xalkori), dabrafenib (Tafinlar), dacomitinib (Vizimpro), daratumumab (Darzalex), daratumumab and hyaluronidase-fihj (Darzalex Faspro), darolutamide (Nubeqa), dasatinib (Sprycel), denileukin diftitox (Ontak), denosumab (Xgeva), dinutuximab (Unituxin), dostarlimab-gxly (Jemperli), durvalumab (Imfinzi), duvelisib (Copiktra), elotuzumab (Empliciti), enasidenib mesylate (Idhifa), encorafenib (Braftovi), enfortumab vedotin-ejfv (Padcev), entrectinib (Rozlytrek), enzalutamide (Xtandi), erdafitinib (Balversa), erlotinib (Tarceva), everolimus (Afinitor), exemestane (Aromasin), fam-trastuzumab deruxtecan-nxki (Enhertu), fedratinib hydrochloride (Inrebic), fulvestrant (Faslodex), gefitinib (Iressa), gemtuzumab ozogamicin (Mylotarg), gilteritinib (Xospata), glasdegib maleate (Daurismo), hyaluronidase-zzxf (Phesgo), ibrutinib (Imbruvica), ibritumomab tiuxetan (Zevalin), idecabtagene vicleucel (Abecma), idelalisib (Zydelig), imatinib mesylate (Gleevec), infigratinib phosphate (Truseltiq), inotuzumab ozogamicin (Besponsa), ipilimumab (Yervoy), isatuximab-irfc (Sarclisa), ivosidenib (Tibsovo), ixazomib citrate (Ninlaro), lanreotide acetate (Somatuline Depot), lapatinib (Tykerb), larotrectinib sulfate (Vitrakvi), lenvatinib mesylate (Lenvima), letrozole (Femara), lisocabtagene maraleucel (Breyanzi), loncastuximab tesirine-lpyl (Zynlonta), lorlatinib (Lorbrena), lutetium Lu 177-dotatate (Lutathera), margetuximab-cmkb (Margenza), midostaurin (Rydapt), mobocertinib succinate (Exkivity), mogamulizumab-kpkc (Poteligeo), moxetumomab pasudotox-tdfk (Lumoxiti), naxitamab-gqgk (Danyelza), necitumumab (Portrazza), neratinib maleate (Nerlynx), nilotinib (Tasigna), niraparib tosylate monohydrate (Zejula), nivolumab (Opdivo), obinutuzumab (Gazyva), ofatumumab (Arzerra), olaparib (Lynparza), olaratumab (Lartruvo), osimertinib (Tagrisso), palbociclib (Ibrance), panitumumab (Vectibix), pazopanib (Votrient), pembrolizumab (Keytruda), pemigatinib (Pemazyre), pertuzumab (Perjeta), pexidartinib hydrochloride (Turalio), polatuzumab vedotin-piiq (Polivy), ponatinib hydrochloride (Iclusig), pralatrexate (Folotyn), pralsetinib (Gavreto), radium 223 dichloride (Xofigo), ramucirumab (Cyramza), regorafenib (Stivarga), ribociclib (Kisqali), ripretinib (Qinlock), rituximab (Rituxan), rituximab and hyaluronidase human (Rituxan Hycela), romidepsin (Istodax), rucaparib camsylate (Rubraca), ruxolitinib phosphate (Jakafi), sacituzumab govitecan-hziy (Trodelvy), seliciclib, selinexor (Xpovio), selpercatinib (Retevmo), selumetinib sulfate (Koselugo), siltuximab (Sylvant), sirolimus protein-bound particles (Fyarro), sonidegib (Odomzo), sorafenib (Nexavar), sotorasib (Lumakras), sunitinib (Sutent), tafasitamab-cxix (Monjuvi), tagraxofusp-erzs (Elzonris), talazoparib tosylate (Talzenna), tamoxifen (Nolvadex), tazemetostat hydrobromide (Tazverik), tebentafusp-tebn (Kimmtrak), temsirolimus (Torisel), tepotinib hydrochloride (Tepmetko), tisagenlecleucel (Kymriah), tisotumab vedotin-tftv (Tivdak), tocilizumab (Actemra), tofacitinib (Xeljanz), tositumomab (Bexxar), trametinib (Mekinist), trastuzumab (Herceptin), tretinoin (Vesanoid), tivozanib hydrochloride (Fotivda), toremifene (Fareston), tucatinib (Tukysa), umbralisib tosylate (Ukoniq), vandetanib (Caprelsa), vemurafenib (Zelboraf), venetoclax (Venclexta), vismodegib (Erivedge), vorinostat (Zolinza), zanubrutinib (Brukinsa), ziv-aflibercept (Zaltrap), or any combination thereof.

In some aspects, the method further comprises obtaining the sample from the subject. In some aspects, the sample comprises a tissue biopsy sample, a liquid biopsy sample, or a normal control. In some aspects, the sample is a liquid biopsy sample and comprises blood, plasma, cerebrospinal fluid, sputum, stool, urine, or saliva. In some aspects, the sample is a liquid biopsy sample and comprises circulating tumor cells (CTCs). In some aspects, the sample is a liquid biopsy sample and comprises cell-free DNA (cfDNA). In some aspects, the cell-free DNA (cfDNA) or a portion thereof comprises circulating tumor DNA (ctDNA). In some aspects, the plurality of nucleic acid molecules comprises a mixture of tumor nucleic acid molecules and non-tumor nucleic acid molecules. In some aspects, the tumor nucleic acid molecules are derived from a tumor portion of a heterogeneous tissue biopsy sample, and the non-tumor nucleic acid molecules are derived from a normal portion of the heterogeneous tissue biopsy sample. In some aspects, the sample comprises a liquid biopsy sample, and wherein the tumor nucleic acid molecules are derived from a circulating tumor DNA (ctDNA) fraction of the liquid biopsy sample, and the non-tumor nucleic acid molecules are derived from a non-tumor, cell-free DNA (cfDNA) fraction of the liquid biopsy sample.

In some aspects, the one or more adapters comprise amplification primers, flow cell adaptor sequences, substrate adapter sequences, or sample index sequences. In some aspects, the captured nucleic acid molecules are captured from the amplified nucleic acid molecules by hybridization to one or more bait molecules. In some aspects, the one or more bait molecules comprise one or more nucleic acid molecules, each comprising a region that is complementary to a region of a captured nucleic acid molecule. In some aspects, amplifying nucleic acid molecules comprises performing a polymerase chain reaction (PCR) amplification technique, a non-PCR amplification technique, or an isothermal amplification technique. In some aspects, the sequencing comprises use of a massively parallel sequencing (MPS) technique, whole genome sequencing (WGS), whole exome sequencing, targeted sequencing, direct sequencing, or Sanger sequencing technique. In some aspects, the sequencing comprises massively parallel sequencing, and the massively parallel sequencing technique comprises next generation sequencing (NGS). In some aspects, the sequencer comprises a next generation sequencer.

In some aspects, one or more of the plurality of sequencing reads overlap one or more gene loci within one or more subgenomic intervals in the sample. In some aspects, the one or more genomic loci comprises between 10 and 20 loci, between 10 and 40 loci, between 10 and 60 loci, between 10 and 80 loci, between 10 and 100 loci, between 10 and 150 loci, between 10 and 200 loci, between 10 and 250 loci, between 10 and 300 loci, between 10 and 350 loci, between 10 and 400 loci, between 10 and 450 loci, between 10 and 500 loci, between 20 and 40 loci, between 20 and 60 loci, between 20 and 80 loci, between 20 and 100 loci, between 20 and 150 loci, between 20 and 200 loci, between 20 and 250 loci, between 20 and 300 loci, between 20 and 350 loci, between 20 and 400 loci, between 20 and 500 loci, between 40 and 60 loci, between 40 and 80 loci, between 40 and 100 loci, between 40 and 150 loci, between 40 and 200 loci, between 40 and 250 loci, between 40 and 300 loci, between 40 and 350 loci, between 40 and 400 loci, between 40 and 500 loci, between 60 and 80 loci, between 60 and 100 loci, between 60 and 150 loci, between 60 and 200 loci, between 60 and 250 loci, between 60 and 300 loci, between 60 and 350 loci, between 60 and 400 loci, between 60 and 500 loci, between 80 and 100 loci, between 80 and 150 loci, between 80 and 200 loci, between 80 and 250 loci, between 80 and 300 loci, between 80 and 350 loci, between 80 and 400 loci, between 80 and 500 loci, between 100 and 150 loci, between 100 and 200 loci, between 100 and 250 loci, between 100 and 300 loci, between 100 and 350 loci, between 100 and 400 loci, between 100 and 500 loci, between 150 and 200 loci, between 150 and 250 loci, between 150 and 300 loci, between 150 and 350 loci, between 150 and 400 loci, between 150 and 500 loci, between 200 and 250 loci, between 200 and 300 loci, between 200 and 350 loci, between 200 and 400 loci, between 200 and 500 loci, between 250 and 300 loci, between 250 and 350 loci, between 250 and 400 loci, between 250 and 500 loci, between 300 and 350 loci, between 300 and 400 loci, between 300 and 500 loci, between 350 and 400 loci, between 350 and 500 loci, or between 400 and 500 loci.

In some aspects, the one or more genomic loci comprise ABL1, ACVR1B, AKT1, AKT2, AKT3, ALK, ALOX12B, AMER1, APC, AR, ARAF, ARFRP1, ARID1A, ASXL1, ATM, ATR, ATRX, AURKA, AURKB, AXIN1, AXL, BAP1, BARD1, BCL2, BCL2L1, BCL2L2, BCL6, BCOR, BCORL1, BCR, BRAF, BRCA1, BRCA2, BRD4, BRIP1, BTG1, BTG2, BTK, CALR, CARD11, CASP8, CBFB, CBL, CCND1, CCND2, CCND3, CCNE1, CD22, CD274, CD70, CD74, CD79A, CD79B, CDC73, CDH1, CDK12, CDK4, CDK6, CDK8, CDKN1A, CDKN1B, CDKN2A, CDKN2B, CDKN2C, CEBPA, CHEK1, CHEK2, CIC, CREBBP, CRKL, CSF1R, CSF3R, CTCF, CTNNA1, CTNNB1, CUL3, CUL4A, CXCR4, CYP17A1, DAXX, DDR1, DDR2, DIS3, DNMT3A, DOT1L, EED, EGFR, EMSY (C11orf30), EP300, EPHA3, EPHB1, EPHB4, ERBB2, ERBB3, ERBB4, ERCC4, ERG, ERRFI1, ESR1, ETV4, ETV5, ETV6, EWSR1, EZH2, EZR, FAM46C, FANCA, FANCC, FANCG, FANCL, FAS, FBXW7, FGF10, FGF12, FGF14, FGF19, FGF23, FGF3, FGF4, FGF6, FGFR1, FGFR2, FGFR3, FGFR4, FH, FLCN, FLT1, FLT3, FOXL2, FUBP1, GABRA6, GATA3, GATA4, GATA6, GID4 (C17orf39), GNA11, GNA13, GNAQ, GNAS, GRM3, GSK3B, H3F3A, HDAC1, HGF, HNF1A, HRAS, HSD3B1, ID3, IDH1, IDH2, IGF1R, IKBKE, IKZF1, INPP4B, IRF2, IRF4, IRS2, JAK1, JAK2, JAK3, JUN, KDM5A, KDM5C, KDM6A, KDR, KEAP1, KEL, KIT, KLHL6, KMT2A (MLL), KMT2D (MLL2), KRAS, LTK, LYN, MAF, MAP2K1, MAP2K2, MAP2K4, MAP3K1, MAP3K13, MAPK1, MCL1, MDM2, MDM4, MED12, MEF2B, MEN1, MERTK, MET, MITF, MKNK1, MLH1, MPL, MRE11A, MSH2, MSH3, MSH6, MST1R, MTAP, MTOR, MUTYH, MYB, MYC, MYCL, MYCN, MYD88, NBN, NF1, NF2, NFE2L2, NFKB1A, NKX2-1, NOTCH1, NOTCH2, NOTCH3, NPM1, NRAS, NT5C2, NTRK1, NTRK2, NTRK3, NUTM1, P2RY8, PALB2, PARK2, PARP1, PARP2, PARP3, PAX5, PBRM1, PDCD1, PDCD1LG2, PDGFRA, PDGFRB, PDK1, PIK3C2B, PIK3C2G, PIK3CA, PIK3CB, PIK3R1, PIM1, PMS2, POLD1, POLE, PPARG, PPP2R1A, PPP2R2A, PRDM1, PRKAR1A, PRKC1, PTCH1, PTEN, PTPN11, PTPRO, QKI, RAC1, RAD21, RAD51, RAD51B, RAD51C, RAD51D, RAD52, RAD54L, RAF1, RARA, RB1, RBM10, REL, RET, RICTOR, RNF43, ROS1, RPTOR, RSPO2, SDC4, SDHA, SDHB, SDHC, SDHD, SETD2, SF3B1, SGK1, SLC34A2, SMAD2, SMAD4, SMARCA4, SMARCB1, SMO, SNCAIP, SOCS1, SOX2, SOX9, SPEN, SPOP, SRC, STAG2, STAT3, STK11, SUFU, SYK, TBX3, TEK, TERC, TERT, TET2, TGFBR2, TIPARP, TMPRSS2, TNFAIP3, TNFRSF14, TP53, TSC1, TSC2, TYRO3, U2AF1, VEGFA, VHL, WHSC1, WHSC1L1, WT1, XPO1, XRCC2, ZNF217, ZNF703, or any combination thereof. In some aspects, the one or more genomic loci comprise ABL, ALK, ALL, B4GALNT1, BAFF, BCL2, BRAF, BRCA, BTK, CD19, CD20, CD3, CD30, CD319, CD38, CD52, CDK4, CDK6, CML, CRACC, CS1, CTLA-4, dMMR, EGFR, ERBB1, ERBB2, FGFR1-3, FLT3, GD2, HDAC, HER1, HER2, HR, IDH2, IL-1β, IL-6, IL-6R, JAK1, JAK2, JAK3, KIT, KRAS, MEK, MET, MSI-H, mTOR, PARP, PD-1, PDGFR, PDGFRα, PDGFRB, PD-L1, PI3Kδ, PIGF, PTCH, RAF, RANKL, RET, ROS1, SLAMF7, VEGF, VEGFA, VEGFB, or any combination thereof.

In some aspects, the method further comprises generating, by the one or more processors, a report indicating the presence or absence of the correlation. In some aspects, the method further comprises transmitting the report to a healthcare provider. In some aspects, the report is transmitted via a computer network or a peer-to-peer connection.

In some aspects, disclosed herein is a method comprising: receiving, using one or more processors, genomic data derived from a sample from a subject; determining, using the one or more processors, a copy number at one or more genomic loci based on the genomic data; determining, using the one or more processors, a distance for each of the one or more genomic loci from a centromere of a chromosome; and determining a correlation, using the one or more processors, wherein the correlation comprises a change in the determined copy number as a function of the determined distances.

In some aspects, the method further comprises detecting, using the one or more processors, a presence or an absence of a disease marker in the sample based on the correlation. In some aspects, the disease marker comprises a Howell-Jolly Body (HJB). In some aspects, the method further comprises diagnosing a disease in the subject based on the presence or the absence of the disease marker in the sample from the subject. In some aspects, the method further comprises treating the subject for the disease based on the presence or the absence of the disease marker in the sample from the subject. In some aspects, the disease comprises asplenia, hyposplenia, or leukopenia. In some aspects, the presence or the absence of HJBs informs a decision regarding treatment of the subject with an immunotherapy. In some aspects, the presence of HJBs informs a contraindication for an immunotherapy.

In some aspects, the correlation is a non-linear correlation. In some aspects, the non-linear correlation is an exponential correlation. In some aspects, the correlation comprises an exponential increase in the copy number as a function of the distance for each of the one or more genomic loci from the centromere of the chromosome. In some aspects, the correlation is a linear correlation. In some aspects, the correlation comprises a linear increase in the copy number as a function of the distance for each of the one or more genomic loci from the centromere of the chromosome. In some aspects, the correlation is a positive correlation. In some aspects, the correlation is positive when the one or more genomic loci are on a q-arm of the chromosome. In some aspects, the correlation is a negative correlation. In some aspects, the correlation is negative when the one or more genomic loci are on a p-arm of the chromosome.

In some aspects, the copy number at the one or more genomic loci comprises a gain or a loss in copy number relative to a baseline copy number. In some aspects, the baseline copy number is 2. In some aspects, the gain or the loss in copy number relative to the baseline copy number is a log-normalized gain or loss in the copy number relative to the baseline copy number. In some aspects, the log-normalized gain or loss in copy number is log-normalized to base 2.

In some aspects, determining the correlation comprises visualizing the change in the determined copy number as a function of the distance for each of the one or more genomic loci from the centromere of the chromosome in a plot of copy number versus chromosomal position. In some aspects, the plot of copy number versus chromosomal position comprises a plot of log-normalized gain or loss in copy number as a function of the distance for each of the one or more genomic loci from the centromere of the chromosome. In some aspects, determining the correlation comprises fitting a regression model to the change in the determined copy number as a function of the distance for each of the one or In some aspects, the fitted regression model comprises a linear regression model. In some aspects, the linear regression model comprises a linear regression of a change in log-normalized gain or loss in the copy number as a function of the distance for each of the one or more genomic loci from the centromere of the chromosome. In some aspects, the correlation is indicative of the presence of HJBs in the sample when the linear regression model comprises a slope greater than a predetermined slope or a y-intercept greater than a predetermined y-intercept. In some aspects, the fitted regression model comprises a non-linear regression model. In some aspects, the non-linear regression model comprises an exponential regression model, a logarithmic In some aspects, the non-linear regression model comprises a non-linear regression of the change in the gain or loss in the copy number, as the function of the determined distances.

In some aspects, the method further comprises performing quality control analysis on the received genomic data. In some aspects, performing the quality control analysis comprises analyzing a tumor cell fraction for the sample, a genome ploidy for the sample, or a size of a subclonal population of the sample containing copy number variants (CNVs). In some aspects, the analyzing the quality control analysis comprises filtering the received genomic data based on the tumor cell fraction for the sample, the genome ploidy for the sample, or the fraction of the subclonal population of the sample containing the CNVs. In some aspects, the determined distances comprise negative or positive values relative to the centromere. In some aspects, the determined distances absolute values of the determined distances relative to the centromere. In some aspects, the received genomic data comprises data for one or more on-target genomic regions and/or one or more off-target genomic regions.

In some aspects, the method further comprises normalizing the received genomic data. In some aspects, the normalizing the received genomic data is normalized based on a synthetic normal set of sequence read count data. In some aspects, the synthetic normal set of sequence read count data is based on a non-subject profile based on a plurality of non-subject normal samples. In some aspects, the normalizing the received genomic data comprises normalizing the received genomic data based on the tumor cell fraction for the sample, the genome ploidy for the sample, or the fraction of the subclonal population of the sample containing the CNVs. In some aspects, the received genomic data comprises sequence read data obtained from next-generation sequencing of the sample. In some aspects, the received genomic data is obtained from a microarray assay, a PCR-based assay, or a probe displacement-based assay.

In some aspects, the method further comprises receiving a histopathology image from a blood sample from the subject; and detecting a HJB from the received histopathology image. In some aspects, wherein detecting the HJB in the sample is used to diagnose or confirm a diagnosis of disease in the subject. In some aspects, the subject previously underwent a splenectomy, a bone marrow transplant, chemotherapy, or radiation therapy. In some aspects, the disease comprises asplenia, hyposplenia, or leukopenia. In some aspects, the disease further comprises sepsis, anemia, immune dysfunction, and/or amyloidosis. In some aspects, the disease is cancer. In some aspects, the cancer is a hematological cancer.

In some aspects, the method further comprises selecting a therapy to administer to the subject based on the determination of the correlation. In some aspects, the method further comprises determining an effective amount of a therapy to administer to the subject based on the determination of the correlation. In some aspects, the method further comprises administering a therapy to the subject based on the determination of the correlation. In some aspects, the therapy is an anti-cancer therapy and comprises chemotherapy, radiation therapy, immunotherapy, a targeted therapy, or surgery. In some aspects, the therapy is a therapy for a disease comprising asplenia, hyposplenia, or leukopenia.

In some aspects, disclosed herein is a method for diagnosing a disease, the method comprising diagnosing that a subject has the disease based on a determination of a correlation between copy number at one or more genomic loci and distance for each of the one or more genomic loci from a centromere of a chromosome for a sample from the subject, wherein the correlation is determined according to any of the methods disclosed herein. In some aspects, disclosed herein is a method of selecting a disease therapy, the method comprising: responsive to determining a correlation for a sample from a subject, selecting a disease therapy for the subject, wherein the correlation is determined according to any of the methods disclosed herein. In some aspects, disclosed herein is a method of treating a disease in a subject, comprising: responsive to determining a correlation for a sample from the subject, administering an effective amount of a disease therapy to the subject, wherein the correlation is determined according to any of the methods disclosed herein.

In some aspects, disclosed herein is a method for monitoring disease progression or recurrence in a subject, the method comprising: determining a first correlation in a first sample obtained from the subject at a first time point according to any of the methods disclosed herein; determining a second correlation in a second sample obtained from the subject at a second time point; and comparing the first correlation to the second correlation, thereby monitoring the disease progression or recurrence. In some aspects, the second correlation for the second sample is determined according to any of the methods disclosed herein. In some aspects, the method further comprises selecting a therapy for the subject in response to the disease progression. In some aspects, the method further comprises administering a therapy to the subject in response to the disease progression. In some aspects, the method further comprises adjusting a disease therapy for the subject in response to the disease progression. In some aspects, the method further comprises adjusting a dosage of the disease therapy or selecting a different disease therapy in response to the disease progression. In some aspects, the method further comprises administering the adjusted disease therapy to the subject. In some aspects, the first time point is before the subject has been administered a disease therapy, and wherein the second time point is after the subject has been administered the disease therapy.

In some aspects, the subject has a disease, is at risk of having a disease, is being routine tested for disease, or is suspected of having a disease. In some aspects, the disease comprises asplenia, hyposplenia, or leukopenia. In some aspects, the disease further comprises sepsis, anemia, immune dysfunction, and/or amyloidosis. In some aspects, the disease is a hematological cancer. In some aspects, the therapy is a therapy for a disease comprising asplenia, hyposplenia, or leukopenia. In some aspects, the therapy comprises chemotherapy, radiation therapy, immunotherapy, a targeted therapy, or surgery.

In some aspects, the method further comprises generating a genomic profile for the subject based on the determination of the correlation. In some aspects, the genomic profile for the subject further comprises results from a comprehensive genomic profiling (CGP) test, a gene expression profiling test, a cancer hotspot panel test, a DNA methylation test, a DNA fragmentation test, an RNA fragmentation test, or any combination thereof. In some aspects, the genomic profile for the subject further comprises results from a nucleic acid sequencing-based test. In some aspects, the method further comprises selecting a therapy, administering a therapy, or applying a therapy to the subject based on the generated genomic profile. In some aspects, the determination of the correlation for the sample is used in making suggested treatment decisions for the subject. In some aspects, the determination of the correlation for the sample is used in applying or administering a treatment to the subject.

In some aspects, disclosed herein is a system comprising: one or more processors; and a memory communicatively coupled to the one or more processors and configured to store instructions that, when executed by the one or more processors, cause the system to: receive, using the one or more processors, genomic data from a sample from a subject; determine, using the one or more processors, a copy number at one or more genomic loci based on the genomic data; determine, using the one or more processors, a distance for each of the one or more genomic loci from a centromere of a chromosome; and determine a correlation, using the one or more processors, wherein the correlation comprises a change in the determined copy number as a function of the determined distances. In some aspects, the system comprises further instructions that, when executed by the one or more processors, cause the system to: detect a presence or an absence of a disease marker in the sample based on the correlation. In some aspects, the disease marker comprises a Howell-Jolly Body (HJB).

In some aspects, the system comprises further instructions that, when executed by the one or more processors, cause the system to: diagnose a disease in the subject based on the presence or the absence of the disease marker in the sample from the subject. In some aspects, the system comprises further instructions that, when executed by the one or more processors, cause the system to: treat the subject for the disease based on presence or the absence of the disease marker in the sample from the subject. In some aspects, the system comprises further instructions that, when executed by the one or more processors, cause the system to: perform quality control analysis on the received genomic data.

In some aspects, disclosed herein is a non-transitory computer-readable storage medium storing one or more programs, the one or more programs comprising instructions, which when executed by one or more processors of a system, cause the system to: receive genomic data from a sample from a subject; determine a copy number at one or more genomic loci based on the genomic data; determine a distance for each of the one or more genomic loci from a centromere of a chromosome; and determine a correlation, wherein the correlation comprises a change in the determined copy number as a function of the determined distances. In some aspects, the non-transitory computer-readable storage medium comprises further instructions that, when executed by the one or more processors, cause the system to: detect a presence or an absence of a disease marker in the sample based on the correlation. In some aspects, the disease marker comprises a Howell-Jolly Body (HJB).

In some aspects, the non-transitory computer-readable storage medium comprises further instructions that, when executed by the one or more processors, cause the system to: diagnose a disease in the subject based on the presence of the HJB in the sample from the subject. In some aspects, the non-transitory computer-readable storage medium comprises further instructions that, when executed by the one or more processors, cause the system to: treat the subject for the disease based on the presence or the absence of the disease marker in the sample from the subject. In some aspects, the non-transitory computer-readable storage medium comprises further instructions that, when executed by the one or more processors, cause the system to: perform quality control analysis on the received genomic data.

It should be appreciated that all combinations of the foregoing concepts and additional concepts discussed in greater detail below (provided such concepts are not mutually inconsistent) are contemplated as being part of the inventive subject matter disclosed herein. In particular, all combinations of claimed subject matter appearing at the end of this disclosure are contemplated as being part of the inventive subject matter disclosed herein.

### INCORPORATION BY REFERENCE

All publications, patents, and patent applications mentioned in this specification are herein incorporated by reference in their entirety to the same extent as if each individual publication, patent, or patent application was specifically and individually indicated to be incorporated by reference in its entirety. In the event of a conflict between a term herein and a term in an incorporated reference, the term herein controls.

### BRIEF DESCRIPTION OF THE DRAWINGS

Various aspects of the disclosed methods, devices, and systems are set forth with particularity in the appended claims. A better understanding of the features and advantages of the disclosed methods, devices, and systems will be obtained by reference to the following detailed description of illustrative embodiments and the accompanying drawings, of which:

FIG. 1 provides a non-limiting example of a process for detecting a Howell-Jolly body (HJB) from genomic (e.g., sequencing) data.

FIG. 2 provides another non-limiting example of a process for detecting a HJB from genomic (e.g., sequencing) data following quality control analysis.

FIG. 3 provides another non-limiting example of a process for detecting a HJB from copy number data for a plurality of genomic loci.

FIG. 4 depicts an exemplary computing device or system in accordance with one or more embodiments of the present disclosure.

FIG. 5 depicts an exemplary computer system or computer network, in accordance with some instances of the systems described herein.

FIG. 6 provides a non-limiting example of correlating copy number with chromosomal positions to detect a HJB. The y-axis depicts the log 2 ratio of copy number variants at each chromosomal position, and each panel along the x-axis depicts a different chromosome of the human genome.

### DETAILED DESCRIPTION

Provided herein are methods and systems for detecting HJBs. Using a characteristic genomic signature of HJBs, the disclosed methods and systems allow for HJB detection from genomic data (e.g., nucleic acid sequencing data) derived from peripheral blood. In some aspects of the present invention, the characteristic genomic signature is a “V-shaped” pattern that reflects a copy number profile associated with the presence of HJBs. A “V-shaped” pattern may be formed by an exponential negative increase in copy number on one arm of a chromosome and an exponential decrease in copy number on the other arm of a chromosome, or a linear negative increase in copy number on one arm of a chromosome and a linear increase in copy number on the other arm of a chromosome.

In some instances, for example, a method is described that comprises receiving, using one or more processors, sequence read data from a sample from a subject; determining, using the one or more processors, a copy number at one or more genomic loci based on the sequence read data; determining, using the one or more processors, a distance for each of the one or more genomic loci from a centromere of a chromosome; and determining a correlation, using the one or more processors, wherein the correlation comprises a change in the determined copy number as a function of the determined distances.

In some instances, the method further comprises detecting, using the one or more processors, a presence or absence of a disease marker in the sample based on the correlation. In some instances, the disease marker comprises a HJB. In some instances, the correlation comprises an exponential increase in the copy number as a function of the distance for each of the one or more genomic loci from the centromere of the chromosome. In some instances, the correlation is a negative correlation when the one or more genomic loci reside on the p-arm of the chromosome. In some instances, the correlation is a positive correlation when the one or more genomic loci reside on the q-arm of the chromosome.

In some instances, for example, a system is described that comprises one or more processors; and a memory communicatively coupled to the one or more processors and configured to store instructions that, when executed by the one or more processors, cause the system to: receive, using the one or more processors, genomic data from a sample from a subject; determine, using the one or more processors, a copy number at one or more genomic loci based on the genomic data; determine, using the one or more processors, a distance for each of the one or more genomic loci from a centromere of a chromosome; and determine a correlation, using the one or more processors, wherein the correlation comprises a change in the determined copy number as a function of the determined distances.

In some instances, for example, a non-transitory computer-readable storage medium storing one or more programs is described, the one or more programs comprising instructions, which when executed by one or more processors of a system, cause the system to: receive genomic data from a sample from a subject; determine a copy number at one or more genomic loci based on the genomic data; determine a distance for each of the one or more genomic loci from a centromere of a chromosome; and determine a correlation, wherein the correlation comprises a change in the determined copy number as a function of the determined distances.

### Definitions

Unless otherwise defined, all of the technical terms used herein have the same meaning as commonly understood by one of ordinary skill in the art in the field to which this disclosure belongs.

As used in this specification and the appended claims, the singular forms “a”, “an”, and “the” include plural references unless the context clearly dictates otherwise. Any reference to “or” herein is intended to encompass “and/or” unless otherwise stated.

“About” and “approximately” shall generally mean an acceptable degree of error for the quantity measured given the nature or precision of the measurements. Exemplary degrees of error are within 20 percent (%), typically, within 10%, and more typically, within 5% of a given value or range of values.

As used herein, the terms “comprising” (and any form or variant of comprising, such as “comprise” and “comprises”), “having” (and any form or variant of having, such as “have” and “has”), “including” (and any form or variant of including, such as “includes” and “include”), or “containing” (and any form or variant of containing, such as “contains” and “contain”), are inclusive or open-ended and do not exclude additional, un-recited additives, components, integers, elements, or method steps.

As used herein, the terms “individual,” “patient,” or “subject” are used interchangeably and refer to any single animal, e.g., a mammal (including such non-human animals as, for example, dogs, cats, horses, rabbits, zoo animals, cows, pigs, sheep, and non-human primates) for which treatment is desired. In particular embodiments, the individual, patient, or subject herein is a human.

The terms “cancer” and “tumor” are used interchangeably herein. These terms refer to the presence of cells possessing characteristics typical of cancer-causing cells, such as uncontrolled proliferation, immortality, metastatic potential, rapid growth and proliferation rate, and certain characteristic morphological features. Cancer cells are often in the form of a tumor, but such cells can exist alone within an animal, or can be a non-tumorigenic cancer cell, such as a leukemia cell. These terms include a solid tumor, a soft tissue tumor, or a metastatic lesion. As used herein, the term “cancer” includes premalignant, as well as malignant cancers.

As used herein, “treatment” (and grammatical variations thereof such as “treat” or “treating”) refers to clinical intervention (e.g., administration of an anti-cancer agent or anti-cancer therapy) in an attempt to alter the natural course of the individual being treated, and can be performed either for prophylaxis during the course of clinical pathology. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis.

As used herein, the term “subgenomic interval” (or “subgenomic sequence interval”) refers to a portion of a genomic sequence.

As used herein, the term “subject interval” refers to a subgenomic interval or an expressed subgenomic interval (e.g., the transcribed sequence of a subgenomic interval).

As used herein, the term “sequence read” is a computationally generated sequence generated by a sequencer to represent a sequence of bases of a single strand of a sequenced fragment. A sequence read can refer to a raw sequence read (e.g., a sequence read as obtained directly from a sequencing instrument), an aligned sequence read (e.g., a sequence read that has been aligned to a reference genome), a single-end sequence read, a paired-end sequence read, a merged sequence read (e.g., a sequence read based on merging a group of overlapping paired-end reads), a consensus sequence read (e.g., a sequence read based on performing error correction on a merged sequence read), a computationally reconstructed sequence read (e.g., a sequence read that has been computationally truncated at the 5′ and/or 3′ end), or any combination thereof.

As used herein, the terms “variant sequence” or “variant” are used interchangeably and refer to a modified nucleic acid sequence relative to a corresponding “normal” or “wild-type” sequence. In some instances, a variant sequence may be a “short variant sequence” (or “short variant”), i.e., a variant sequence of less than about 50 base pairs in length.

The terms “allele frequency” and “allele fraction” are used interchangeably herein and refer to the fraction of sequence reads corresponding to a particular allele relative to the total number of sequence reads for a genomic locus.

The terms “variant allele frequency” and “variant allele fraction” are used interchangeably herein and refer to the fraction of sequence reads corresponding to a particular variant allele relative to the total number of sequence reads for a genomic locus.

The section headings used herein are for organizational purposes only and are not to be construed as limiting the subject matter described.

### Methods for Detecting Howell-Jolly Bodies

Erythrocytes can contain Howell-Jolly bodies (HJBs), which are nuclear remnants. Because HJBs contain DNA, genomic sequencing technologies can be used to detect the presence of HJBs in samples. Although erythrocytes and HJBs are normally released into the peripheral blood, subjects with healthy splenic function filter out HJBs leaving them undetectable in typical blood analyses. However, where splenic function is reduced or absent, HJBs can persist in peripheral blood. A characteristic genomic signature can be observed following sequencing of liquid samples containing peripheral blood with persistent HJBs, making it possible to detect HJBs from genomic data.

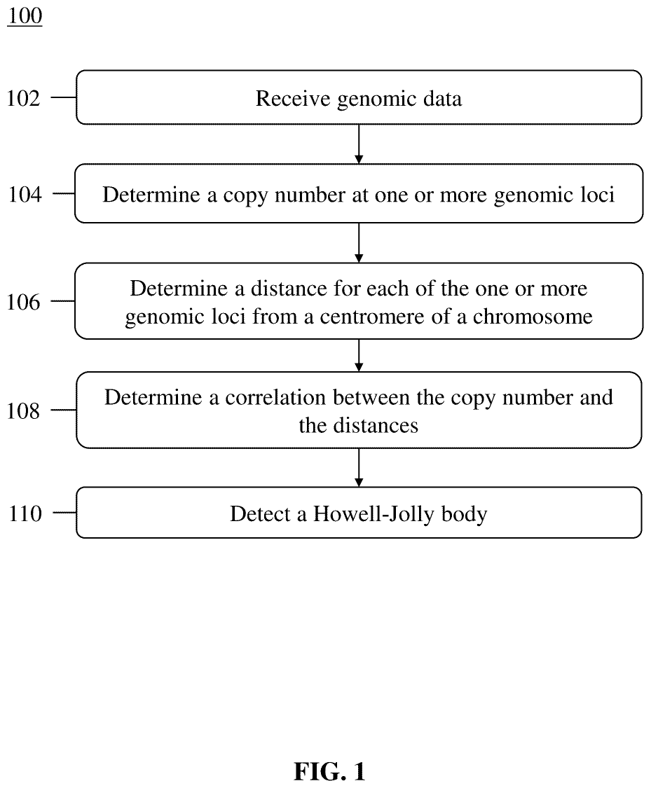

FIG. 1 provides a non-limiting exemplary schematic showing general process 100 for detecting a HJB from genomic data. The method for detecting a HJB can include: receiving genomic data, e.g., sequence read data (102); determining a copy number at one or more genomic loci (104); determining a distance for each of the one or more genomic loci from a centromere of a chromosome (106); determining a correlation between the copy number and the distances (108); and detecting a HJB (110).

At step 102 in FIG. 1, genomic data, e.g., sequence read data, is received. Genomic data can be derived from a sample from a subject. The subject can be an individual who is known to have a disease, suspected to have a disease, at risk of having a disease, is being routine tested for a disease, or known to not have a disease. The type of disease can be, for example, a disease that is known to impact splenic function, such as a disease that results in asplenia or hyposplenia. Asplenia is well-understood to constitute absent splenic function and can be caused by the anatomical absence of the spleen (e.g., following splenectomy or due to congenital absence) or other conditions resulting in splenic abnormalities. Hyposplenia is well-understood to constitute reduced splenic function, and can be caused by conditions such as, but not limited to, hematological cancer (e.g., lymphoma, leukemia), heme-related disease (e.g., sickle cell anemia, megaloblastic anemia, severe hemolysis, myelophthisic anemia), immunological disease (e.g., sarcoidosis, autoimmune disease), gastrointestinal disease (e.g., celiac disease), amyloidosis, hepatic disease (e.g., alcoholic liver disease, hepatic cirrhosis, chronic ciral hepatitis, portal hypotension, primary biliary cirrhosis), infectious disease (e.g., sepsis) rheumatological disease, and iatrogenic disease.

In the disclosed methods and systems, the subject can be an individual who is known to have, suspected to have, or known to not have any of the above diseases. The subject can also be an individual who previously underwent a splenectomy, a bone marrow transplant, chemotherapy, or radiation therapy. The subject may have a condition that leads to low white blood cell counts (i.e., leukopenia, neutropenia), for example, the subject may have an immunodeficiency or may be undergoing immunosuppressive treatment that reduces white blood cell counts. The presence of white blood cells in peripheral blood inhibits HJB detection when using bulk sequencing, thus, a high HJB to white blood cell count ratio in subjects can be leveraged for HJB detection in the disclosed methods and systems.

The sample from the subject can be a tissue biopsy sample, a liquid biopsy sample, or a normal control. In some aspects, the sample can be a liquid biopsy sample that comprises blood, plasma, cerebrospinal fluid, sputum, stool, urine, or saliva. In some aspects, the liquid biopsy sample is a blood sample that comprises circulating tumor cells, cfDNA, and/or ctDNA. In some aspects, the sample is pre-treated to optimize downstream processing. For example, certain genomic pathologies (e.g., aneuploidy, segmental copy number alterations, the presence of ctDNA, leukemic DNA inclusions in erythrocytes such as atypical HJBs) may co-exist in samples with HJBs. In these instances, pre-treatment of the samples can be used to enhance HJB detection and mitigate any focal alteration of the HJB signature. For example, white blood cells or tumor cells may be removed from a sample to enhance HJB detection.

The sample from the subject may be sequenced to generate genomic data. Genomic data can comprise sequence read data obtained from sequencing the sample. Multiple sequencing approaches may be used to obtain the genomic data from the sample. For example, a massively parallel sequencing (MPS) technique, whole genome sequencing (WGS), whole exome sequencing, targeted sequencing, direct sequencing, or Sanger sequencing technique may be used. In some aspects, the sequencing comprises massively parallel sequencing. In some aspects, the sequencing comprises a next generation sequencing approach. Genomic data can also comprise gene expression data. In some aspects, the received genomic data is obtained from a microarray assay, a PCR-based assay, or a probe displacement-based assay such as a Taqman-based assay (e.g., RT-PCR).

In some aspects, the received genomic data may be normalized to control data, which may be described as a synthetic normal set of data. In some aspects, the normalization is based on a synthetic normal set of sequence read count data. In some aspects, the synthetic normal set of sequence read count data is based on a non-subject profile based on a plurality of non-subject normal samples. The normalization of the received genomic data can include normalizing for purity or ploidy in the sample. Methods of data normalization are provided in PCT/US2023/069150 which is incorporated herein by reference.

At step 104 in FIG. 1, a copy number is determined at one or more genomic loci. A copy number is the number of copies of a gene in a genome. A copy number can be examined at a specific genomic locus, or at more than one genomic loci, in reference to a specific gene or more than one gene, or in reference to a specific genomic position or more than one genomic position. Multiple copies of a gene can be referred to as copy number variants (CNVs). Copy numbers may vary between different genes and for different individuals. This variation can be referred to as copy number variation. CNVs can have different biological roles in a genome and may indicate the presence of disease. In some instances, CNVs may be present in a genome due to insertions, deletions, duplications, or other types of genetic variants.

In some aspects of the present invention, the copy number at one or more genomic loci is at least 0, at least 1, at least 2, at least 3, at least 4, at least 5, at least 6, at least 7, at least 8, at least 9, at least 10, at least 20, at least 30, at least 40, at least 50, at least 60, at least 70, at least 80, at least 90, at least 100, at least 200, at least 300, at least 400, or at least 500. In some aspects, the copy number at a genomic locus is 1 and the locus is heterozygous having 2 non-identical alleles at that locus. In some aspects, the copy number at a genomic locus is 2 and the locus is homozygous having 2 identical alleles at that locus. In some aspects, the copy number at one or more genomic loci is an absolute value that is independently determined. In some aspects, the copy number at one or more genomic loci comprises a relative value compared to a baseline copy number. The copy number can comprise a gain or loss in copy number relative to a baseline copy number. The baseline copy number can represent the ploidy of the genome of the subject from which the genomic data was derived.

For example, in a human genome, the baseline copy number can be 2. A copy number of 2 can represent a genome within a diploid cell, i.e., a cell that has 2 sets of chromosomes. Individual homozygous genes within a diploid genome are often expected to exist in 2 copies. To account for any genetic variation in copy number, the copy number at one or more genomic loci may comprise a gain or loss in copy number relative to 2. For example, for a locus that has experienced a genetic duplication event, the copy number of a gene at the locus may be 3, comprising a gain of 1 relative to the baseline copy number of 2. For a locus that has experienced a genetic deletion event or for a locus that is heterozygous, the copy number of a gene at the locus may be 1, comprising a loss of 1 relative to the baseline copy number of 2. In some instances, the baseline copy number is 2 at all genomic loci within the genome of the subject. In some instances, the baseline copy number is 2 at some of the genomic loci within the genome of the subject. In some instances, the baseline copy number is at least 0, at least 1, at least 2, at least 3, at least 4, at least 5, at least 6, at least 7, at least 8, at least 9, or at least 10, at least 20, at least 30, at least 40, at least 50, at least 60, at least 70, at least 80, at least 90, at least 100, or more than 100 at some of the genomic loci within the genome of the subject.

In any of the above instances, the determined copy number can be a copy number value. For example, the copy number value can be the copy number at one or more genomic loci that is determined and visualized as a log-normalized ratio, or a log-normalized to base 2 ratio. For example, the gain or loss in copy number relative to the baseline copy number can be a log-normalized gain or loss in the copy number relative to the baseline copy number, or a log-normalized to base 2 gain or loss in the copy number relative to the baseline copy number. In some embodiments, the copy number value is a log 2 ratio of sequencing reads, for example, the number of sequencing reads from a sample at a given genomic locus as compared to the number of sequencing reads from a sample at a reference genomic locus. As above, the copy number value can be determined using genomic data that has been normalized for tumor purity and/or ploidy.

In some embodiments, the copy number value is determined using copy number modeling with a ratio equation determined from sequencing read coverage, i.e., a ratio of sequencing read coverage. Sequencing read coverage can be determined, e.g., as set forth in Equation 1, wherein rcᵢ is read coverage at a given genomic region i, γᵢ is a proportionality constant at a given genomic region i, ρ is tumor purity, N is number of cells, and CNᵢ is copy number at a given genomic region i.

rcᵢ=γᵢρNCNᵢ Equation 1: Model read coverage with physical parameters

In some embodiments, the ratio equation used to determine the copy number value is formed by dividing the modeled test coverage by the reference coverage, e.g., as set forth in Equation 2, wherein R is the ratio, rc_{total,i} is the modeled test coverage, and rc_{total,n} is the reference coverage.

$$Equation 2$$

$$Ratio equation for modeled test coverage and reference coverage$$

$$R = \frac{r {c}_{total , t}}{r {c}_{total , n}}$$

In some embodiments, the ratio equation may be transformed to a different representation, e.g., as set forth in Equation 3, wherein R is the ratio,

$$( \frac{{γ}_{t}}{{γ}_{r}} \frac{{N}_{t}}{{N}_{r}} )$$

is a proportionality constant for normalization where t is a tumor sample and r is a reference sample, ρ is tumor purity, N is number of cells, and CN is copy number.

$$Transformed ratio equation \qquad Equation 3$$

$$R = ( \frac{{γ}_{t}}{{γ}_{r}} \frac{{N}_{t}}{{N}_{r}} ) \frac{ρ C N + 2 ( 1 - ρ )}{2}$$

In some embodiments, a statistic of the ratio can be defined (e.g., mean of the ratio, mode of the ratio, median of the ratio), which is represented by an appropriate distribution (e.g., log-normal distribution, ratio distribution), where the statistic is the central value to which the measured ratio data will be shifted, for example, as set forth in Equation 4, wherein <R>_{loci} is the set of ratios at all genomic loci, μ is the mean ratio, and σ is the log-normal distribution representing the variance of the read coverage ratio. In some embodiments, the measured <R>_{loci} is used as a normalizing value.

<R>_{loci}≡e^{μ+σ}^{²}^{/2} Equation 4: Mean ratio from a log-normal distribution

In some embodiments, the proportionality constant γᵢ (see Equation 1) is used to constrain the statistic of the ratio R. For example, with the mean ratio μ set to 0, Equation 5 relates the proportionality constants and the modeled parameters, wherein

$$( \frac{{γ}_{t}}{{γ}_{r}} \frac{{N}_{t}}{{N}_{r}} )$$

is a proportionality constant for normalization where t is a tumor sample and r is a reference sample, ρ is tumor purity, N is number of cells, CN is copy number, and ψ is ploidy.

$$Equation 5$$

$$\begin{matrix}Ratio equation relating the proportionality \\ constants and modeled parameters\end{matrix}$$

$$< R {>}_{l o c i} = ( \frac{{γ}_{t}}{{γ}_{r}} \frac{{N}_{t}}{{N}_{r}} ) \frac{1}{N} {\sum }_{i}^{N} \frac{ρ C {N}_{i} + 2 ( 1 - p )}{2} = ( \frac{{γ}_{t}}{{γ}_{r}} \frac{{N}_{t}}{{N}_{r}} ) \frac{ρ ψ + 2 ( 1 - ρ )}{2} = {e}^{{σ}^{2} / 2}$$

$$( \frac{{γ}_{t}}{{γ}_{r}} \frac{{N}_{t}}{{N}_{r}} ) = \frac{2 {e}^{{σ}^{2} / 2}}{ρ ψ + 2 ( 1 - ρ )}$$

In some embodiments, the normalized modeled ratio equation used to determine the copy number value is set forth in Equation 6, wherein the equation is based on the proportionality constants, modeled parameters, and constraint determined as described above, and wherein R is the ratio, ρ is tumor purity, CN is copy number, ψ is ploidy, and σ is the distribution representing the variance of the read coverage ratio.

$$Normalized model ratio equation \qquad Equation 6$$

$$R = \frac{ρ C N + 2 ( 1 - ρ )}{ρ ψ + 2 ( 1 - ρ )} {e}^{{σ}^{2} / 2}$$

In some embodiments, a statistic of the log 2 ratio can be defined (e.g., mean of the log 2 ratio) and set to 0, i.e., <log₂(R)>_{loci}≡μ, where μ=0, and wherein this constraint provides another normalized modified ratio equation that can be used to determine the copy number value, as set forth in Equation 7, wherein R̅_{geom} is the average taken in log space and is called, rather than the arithmetic mean, the geometric mean.

$$Normalized model ratio equation \qquad Equation 7$$

$${R}_{i} = \frac{ρ {CN}_{i} + 2 ( 1 - ρ )}{{\overline{R}}_{geom}} = \frac{ρ {CN}_{i} + 2 ( 1 - ρ )}{{Π}_{j} ( {pCN}_{j} + 2 {( 1 - ρ )}^{1 / N}}$$

At step 106 in FIG. 1, a distance is determined for each of the one or more genomic loci from a centromere of a chromosome. A centromere is a fixed position on a chromosome and is unique to each chromosome. Centromeres do not contain genes and are often flanked by genomic regions comprising gene deserts. Because of this, when samples are sequenced to generate genomic data, there is often no data generated very close to the centromere.

It is well-understood in the art that a centromere is a region on a chromosome that separates the p-arm (short arm) of the chromosome and the q-arm (long arm) of the chromosome. However, the position of the centromere for each chromosome may be different depending on the type of chromosome. Human chromosomes can be metacentric or acrocentric. A metacentric chromosome has a centromere that is positioned approximately in the middle of the chromosome's distal ends, separating the p-arm and the q-arm of the chromosome. An acrocentric chromosome has an off-center centromere that is positioned near one of the chromosome's distal ends, separating the p-arm and the q-arm of the chromosome. The p-arm of an acrocentric chromosome is typically even smaller than the p-arm of a metacentric chromosome.

Genomic loci may be in any position within metacentric or acrocentric chromosomes. In the disclosed methods and systems, a genomic locus may be located on the p-arm of a chromosome or the q-arm of a metacentric or acrocentric chromosome. On either the p-arm or the q-arm, a genomic locus may be located anywhere up to the telomere of a chromosome. In some aspects, a genomic locus is located about 2,500 base pairs away from a centromere on either side, that is, on either the p-arm or the q-arm of a chromosome. In some aspects, a genomic locus is located more than 2,500 base pairs away from a centromere on either the p-arm or the q-arm of a chromosome.

The determined distance between a genomic locus and a centromere can be a physical chromosomal or genomic distance described in base pairs. For example, a determined distance between a genomic locus and a centromere can be between about 2,500 base pairs and 5,000 base pairs, between about 5,000 base pairs and 7,500 base pairs, between about 7,500 base pairs and 10,000 base pairs, between about 10,000 base pairs and 50,000 base pairs, between about 50,000 base pairs and 100,000 base pairs, between about 100,000 base pairs and 200,000 base pairs, between about 200,000 base pairs and 300,000 base pairs, between about 300,000 base pairs and 400,000 base pairs, between about 400,000 base pairs and 500,000 base pairs, between about 500,000 base pairs and 1,000,000 base pairs, between about 1,000,000 base pairs and 10,000,000 base pairs, between about 10,000,000 base pairs and 100,000,00 base pairs, or more than 100,000,000 base pairs. In some aspects, the determined distance is a scalar distance representing the number of base pairs between a genomic locus and a centromere. In some aspects, the determined distance is a vector distance representing the number of base pairs between a genomic locus and a centromere and the relative position of the genomic locus to the centromere. For example, the determined distance may be a negative value or a positive value relative to the centromere. A negative value may mean that the genomic locus is located on one side of a centromere and a positive value may mean that the genomic locus is located on the other side of a centromere. The determined distance may also be an absolute value relative to the centromere representing only the physical base pair distance of a genomic locus from a centromere.

At step 108 in FIG. 1, a correlation is determined between the copy number and the distances. The correlation can comprise a change in copy number as determined at step 104 in FIG. 1 as a function of distance as determined at step 106 in FIG. 1. In some aspects, the correlation is a non-linear correlation. In some aspects, the non-linear correlation is an exponential correlation. An exponential correlation can comprise an exponential increase in copy number as a function of the distance for each genomic locus from the centromere of a chromosome. For example, as the distance from the centromere of a chromosome increases, the copy number at each genomic locus may exponentially increase. In some aspects, the correlation is a linear correlation. A linear correlation can comprise a linear increase in copy number as a function of the distance for each genomic locus from the centromere of a chromosome. For example, as the distance of the centromere of a chromosome increases, the copy number at each genomic locus may show a linear increase. In some aspects, the correlation is positive. In some aspects, the correlation is negative. The correlation may be non-linear and positive, non-linear and negative, exponential and positive, exponential and negative, liner and positive, or linear and negative.

In some aspects, the correlation can be used to identify a “V-shaped” pattern in the received genomic data. A “V” may be observed in the genomic data surrounding a centromere, with a negative correlation on one side of the centromere, and a positive correlation on the other side of the centromere. The negative correlation can be on the p-arm of the centromere and the positive correlation can be on the q-arm of the centromere. For example, in some aspects, the correlation is negative when the one or more genomic loci for which distance is being determined are located on the p-arm of the chromosome, and the correlation is positive when the one or more genomic loci for which distance is being determined are located on the q-arm of the chromosome. On either side of the chromosome, for the negative or positive correlation may be non-linear, exponential, or linear.

In some aspects, the correlation can be used to identify the absence of a “V-shaped” pattern in the received genomic data. For example, there may be a positive correlation on both sides of the centromere, or a negative correlation on both sides of the centromere. Alternatively, the negative correlation can be on the q-arm of the centromere and the positive correlation can be on the p-arm of the centromere. For example, in some aspects, the correlation is negative when the one or more genomic loci for which distance is being determined are located on the q-arm of the chromosome, and the correlation is positive when the one or more genomic loci for which distance is being determined are located on the p-arm of the chromosome. On either side of the chromosome, for the negative or positive correlation may be non-linear, exponential, or linear.

Each chromosome in a genome can have a unique set of correlations. In some aspects, every chromosome in a genome has a similar set of correlations. For example, every chromosome in a genome may show a characteristic “V-shaped” pattern. Alternatively, only chromosomes in a genome may show a “V-shaped” pattern while others have different patterns depending on the relationship between copy number and genomic distance within the chromosome.

Determining the correlation can comprise visualizing the change in the determined copy number as a function of the distance for each of the one or more genomic loci from the centromere of the chromosome in a plot of copy number versus chromosomal position. In some instances, a plot of copy number versus chromosomal position can be used to identify the “V-shaped” patterns as described above. In some instances, the plot comprises a plot of log-normalized gain or loss in copy number as a function of the distance for each of the one or more genomic loci from the centromere of the chromosome.

At step 110 in FIG. 1, a HJB is detected. A HJB can be detected based on a “V-shaped” pattern in the received genomic data, based on the correlations as determined at step 108 in FIG. 1. For example, a HJB can be detected from an exponential negative correlation between copy number and distance from the centromere on the p-arm of the chromosome and an exponential positive correlation between copy number and distance from the centromere on the q-arm of the chromosome. Alternatively, a HJB can be detected from a linear negative correlation between copy number and distance from the centromere on the p-arm of the chromosome and a linear positive correlation between copy number and distance from the centromere on the q-arm of the chromosome.

HJBs can be markers of disease. The detection of the HJB at step 110 can mean that HJBs are present and persistent in the peripheral blood, which may signify splenic dysfunction. The detection of a HJB may be used to diagnose a disease in the subject based on the presence of the HJBs, and to treat the subject for the disease. The presence or absence of HJBs can inform a decision regarding treatment of the subject with an immunotherapy, wherein the presence of HJBs can inform a contraindication for an immunotherapy. The presence of HJBs as identified from the disclosed methods can also be validated by receiving a histopathology image from a blood sample and evaluating the image for the presence of HJBs.

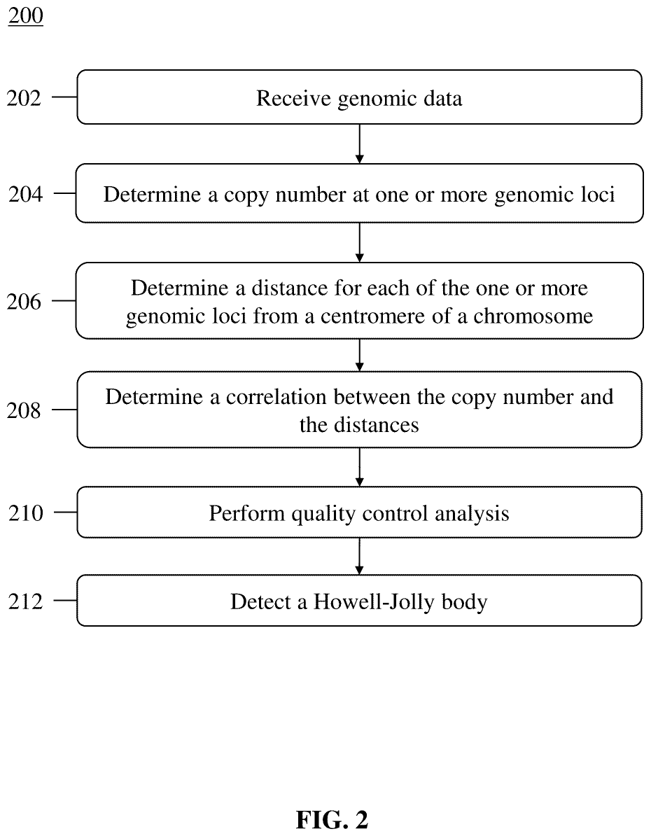

FIG. 2 provides another non-limiting exemplary schematic showing general process 200 for detecting a HJB from genomic data following quality control analysis. The method for detecting a HJB can include: receiving genomic data (202); determining a copy number at one or more genomic loci (204); determining a distance for each of the one or more genomic loci from a centromere of a chromosome (206); determining a correlation between the copy number and the distances (208); performing quality control analysis (210); and detecting a HJB (212).

At step 202 in FIG. 2, genomic data (e.g., sequence read data) is received. Genomic data can be received from a sample from a subject as described at step 102 in FIG. 1. At step 204 in FIG. 2, a copy number is determined at one or more genomic loci. Copy numbers can be determined as described at step 104 in FIG. 1. At step 206 in FIG. 2, a distance is determined for each of the one or more genomic loci from a centromere of a chromosome. Distances can be determined as described at step 106 in FIG. 1. At step 208 in FIG. 2, a correlation is determined between the copy number and the distances. Correlations can be determined as described at step 108 in FIG. 1.

At step 210 in FIG. 2, quality control analysis is performed. Quality control analysis can be done to further process the received genomic data and increase the accuracy and sensitivity of the method in detecting HJBs. Certain features of samples obtained from subjects can lead to false positives or other inaccuracies in data analysis, such as, but not limited to, the purity of a tumor cell fraction, the ploidy of the sample, or the fraction of the subclonal population of the sample containing the CNVs (e.g., the percentage of tumor cells in a sample exhibiting the CNVs among the tumor cell population). Quality control analysis can include filtering out received genomic data associated with any of these features to decrease the likelihood of false positives or other inaccuracies in HJB detection.

For example, a sample obtained from a subject having cancer may comprise a tumor cell fraction. An insufficient purity of the fraction may create genomic data and characteristic genomic signatures that are indiscernible from signatures associated with HJB. Therefore, quality control analysis can include analyzing a tumor cell fraction for a sample and filtering the received genomic data based on the purity of the tumor cell fraction. The received genomic data can then be filtered using a specific cutoff describing a numeric value of the purity of the tumor cell fraction.

A sample obtained from a subject may comprise aneuploidy. For example, one or more cells obtained from a sample from a subject may have an abnormal number of chromosomes, deviating from the typical diploid status of the human genome. Aneuploidy may create genomic signatures that mirror copy number variation not associated with HJB. Therefore, quality control analysis can include analyzing received genomic data for the presence of aneuploidy and filtering the received genomic data based on the ploidy of the sample. The received genomic data can then be filtered using a specific cutoff relating to the ploidy of the sample.

A sample obtained from a subject may also comprise CNVs in only a subset of a subpopulation. For example, a fraction or percentage of tumor cells in a sample may exhibit CNVs among the tumor cell population. In these instances, quality control analysis can include analyzing the data to limit calling to only high-confidence positives for CNVs in the subpopulation.

At step 212 in FIG. 2, a HJB is detected. A HJB can be detected as described for step 110 in FIG. 1.

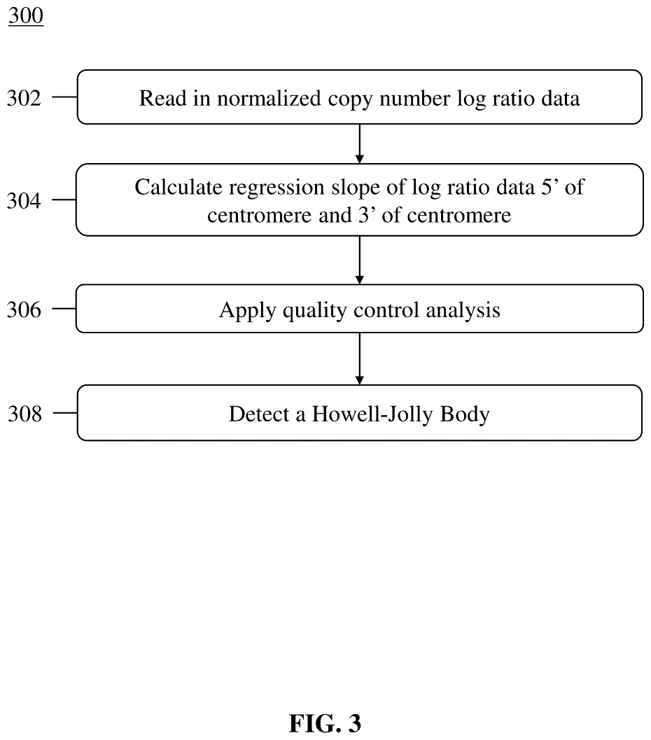

FIG. 3 provides another non-limiting exemplary schematic showing general process 300 for detecting a HJB from genomic data, i.e., in plots of copy number data as a function of the distance of a given genomic locus that exhibits copy number variation from the centromere of the corresponding chromosome. The method for detecting a HJB can include: reading in normalized copy number log ratio data (302); calculating the regression slope of log ratio data 5′ of centromere and 3′ of centromere (304); applying quality control analysis (306); and detecting a HJB (308).

At step 302 in FIG. 3, normalized copy number log ratio data is read in. This data can be generated from genomic data received from a sample from a subject as described for step 102 in FIG. 1. A copy number can be determined at one or more genomic loci and then normalized as described for step 104 in FIG. 1. The normalized copy number log ratio can be calculated by log-normalizing to base 2 the gain or loss in copy number relative to the baseline copy number.

At step 304 in FIG. 3, the regression slope of log ratio data 5′ of the centromere and 3′ of the centromere is calculated. The regression slope is determined by examining the correlation between log ratio data and distance from the centromere in the 5′ or 3′ direction. For a metacentric or acrocentric chromosome, the 5′ direction from the centromere is the p-arm, and the 3′ direction from the centromere is the q-arm. To calculate the regression slope, distances can be determined from the centromere for genomic loci in the 5′ direction on the p-arm and for genomic loci in the 3′ direction on the q-arm, as described for step 106 in FIG. 1. The distance can be a physical chromosomal or genomic distance described in base pairs. A distance in the 5′ or 3′ direction can be determined for each of one or more genomic loci located on either the p-arm or the q-arm of each chromosome.

The normalized copy number log ratio as determined at step 302 in FIG. 3 can be examined as a function of the determined distances for the genomic loci. The relationship between normalized copy number log ratio and determined distance can be represented as a correlation that is non-linear, non-linear and exponential, or linear, as described for step 108 in FIG. 1. Determining the correlation can comprise visualizing the change in the normalized copy number log ratio as a function of the distance in the 5′ or 3′ direction from the centromere of the chromosome in a plot of normalized copy number log ratio versus chromosomal position. In some instances, this type of plot can be used to identify the “V-shaped” patterns as described above.

The “V-shaped” pattern can also be described with the determination of slopes based on the correlations. Determining the correlation can comprise fitting a regression model to the change in the normalized copy number log ratio as a function of the distances for genomic loci from the centromere of the chromosome. In some aspects, the fitted regression model comprises a linear regression model, indicating a linear regression of a change in the normalized copy number log ratio as a function of the distances for genomic loci from the centromere of the chromosome. In some aspects, the fitted regression model is a non-linear regression model, such as an exponential regression model, a logarithmic regression model, or a logistic regression model.

At step 306 in FIG. 3, quality control analysis is performed. Quality control analysis can be done to further process the received genomic data and increase the accuracy and sensitivity of the method in detecting HJBs. Certain features of samples obtained from subjects can lead to false positives or other inaccuracies in data analysis, such as, but not limited to, the purity of a tumor cell fraction, genome ploidy or aneuploidy, or subclonal population size. Quality control analysis can include filtering out received genomic data associated with any of these features to decrease the likelihood of false positives or other inaccuracies in HJB detection.

For example, a sample obtained from a subject having cancer may comprise a tumor cell fraction. An insufficient purity of the fraction may create genomic data and characteristic genomic signatures that are indiscernible from signatures associated with HJB. Therefore, quality control analysis can include analyzing a tumor cell fraction for a sample and filtering the received genomic data based on the purity of the tumor cell fraction. The received genomic data can then be filtered using a specific cutoff describing a numeric value of the purity of the tumor cell fraction.

A sample obtained from a subject may comprise aneuploidy. For example, one or more cells obtained from a sample from a subject may have an abnormal number of chromosomes, deviating from the typical diploid status of the human genome. Aneuploidy may create genomic signatures that mirror copy number variation not associated with HJB. Therefore, quality control analysis can include analyzing received genomic data for the presence of aneuploidy and filtering the received genomic data based on the ploidy of the sample. The received genomic data can then be filtered using a specific cutoff relating to the ploidy of the sample.

At step 308 in FIG. 3, a HJB is detected. A HJB can be detected based on a “V-shaped” pattern in the received genomic data, as described at step 108 in FIG. 1 or based on the regression slopes as described at step 304 in FIG. 3. For example, a HJB can be detected by a linear regression model comprising a slope greater than a predetermined slope or a y-intercept greater than a predetermined y-intercept. The predetermined slope or predetermined y-intercept may be determined depending on the purity of a sample as assessed at step 306 in FIG. 3, the number of genes tested on the p-arm and the q-arm, and/or the normalization of the log ratio data as determined at step 304 in FIG. 3. In any of FIGS. 1-3, a visual, computational, or mathematical approach can be used to detect a HJB. For example, a HJB can be detected visually by examining a “V-shaped” pattern in the received genomic data, or computationally or mathematically using any of the correlation and/or regression-based approaches described herein.

Process 100, 200 or 300 can be performed, for example, using one or more electronic devices implementing a software platform. In some examples, process 100, 200 or 300 is performed using a client-server system, and the blocks of process 100, 200 or 300 are divided up in any manner between the server and a client device. In other examples, the blocks of process 100, 200 or 300 are divided up between the server and multiple client devices. Thus, while portions of process 100, 200 or 300 are described herein as being performed by particular devices of a client-server system, it will be appreciated that process 100, 200 or 300 is not so limited. In other examples, process 100, 200 or 300 is performed using only a client device or only multiple client devices. In process 100, 200 or 300, some blocks are, optionally, combined, the order of some blocks is, optionally, changed, and some blocks are, optionally, omitted. In some examples, additional steps may be performed in combination with the process 100, 200 or 300. Accordingly, the operations as illustrated (and described in greater detail below) are exemplary by nature and, as such, should not be viewed as limiting.

In some instances, the panel of genomic loci (comprising copy number variants) for which distances are determined in any of the methods described herein may comprise at least 1, at least 2, at least 3, at least 4, at least 5, at least 6, at least 7, at least 8, at least 9, at least 10, at least 20, at least 30, at least 40, at least 50, at least 60, at least 70, at least 80, at least 90, at least 100, at least 500, at least 1,000, at least 5,000, at least 10,000, at least 50,000, at least 100,000, at least 500,000, or more than 1,000,000 genomic loci (e.g., gene loci). In some instances, the panel of genomic loci may comprise between about 10 genomic loci to about 1,000 genomic loci. In some instances, the panel of genomic loci may comprise between 10 and 50 genomic loci, between 10 and 100 genomic loci, between 10 and 150 genomic loci, between 10 and 200 genomic loci, between 10 and 250 genomic loci, between 10 and 300 genomic loci, between 10 and 350 genomic loci, between 10 and 400 genomic loci, between 10 and 450 genomic loci, between 10 and 500 genomic loci, between 10 and 550 genomic loci, between 10 and 600 genomic loci, between 10 and 650 genomic loci, between 10 and 700 genomic loci, between 10 and 750 genomic loci, between 10 and 800 genomic loci, between 10 and 850 genomic loci, between 10 and 900 genomic loci, between 10 and 950 genomic loci, between 10 and 1000 genomic loci, between 100 and 150 genomic loci, between 100 and 200 genomic loci, between 100 and 250 genomic loci, between 100 and 300 genomic loci, between 100 and 350 genomic loci, between 100 and 400 genomic loci, between 100 and 450 genomic loci, between 100 and 500 genomic loci, between 100 and 550 genomic loci, between 100 and 600 genomic loci, between 100 and 650 genomic loci, between 100 and 700 genomic loci, between 100 and 750 genomic loci, between 100 and 800 genomic loci, between 100 and 850 genomic loci, between 100 and 900 genomic loci, between 100 and 950 genomic loci, or between 100 and 1,000 genomic loci. In some instances, the panel of genomic loci may comprise between about 1,000 genomic loci to about 5,000 genomic loci, between about 5,000 genomic loci to about 10,000 genomic loci, between about 10,000 genomic loci to about 50,000 genomic loci, between about 50,000 genomic loci to about 100,000 genomic loci, between about 100,000 genomic loci to about 500,000 genomic loci, or between about 500,000 genomic loci to about 1,000,000 genomic loci.

In some instances, the panel of genomic loci (comprising copy number variants) for which distances are determined in any of the disclosed methods may comprise genomic loci located in the ABL1, ACVR1B, AKT1, AKT2, AKT3, ALK, ALOX12B, AMER1, APC, AR, ARAF, ARFRP1, ARID1A, ASXL1, ATM, ATR, ATRX, AURKA, AURKB, AXIN1, AXL, BAP1, BARD1, BCL2, BCL2L1, BCL2L2, BCL6, BCOR, BCORL1, BCR, BRAF, BRCA1, BRCA2, BRD4, BRIP1, BTG1, BTG2, BTK, CALR, CARD11, CASP8, CBFB, CBL, CCND1, CCND2, CCND3, CCNE1, CD22, CD274, CD70, CD74, CD79A, CD79B, CDC73, CDH1, CDK12, CDK4, CDK6, CDK8, CDKN1A, CDKN1B, CDKN2A, CDKN2B, CDKN2C, CEBPA, CHEK1, CHEK2, CIC, CREBBP, CRKL, CSF1R, CSF3R, CTCF, CTNNA1, CTNNB1, CUL3, CUL4A, CXCR4, CYP17A1, DAXX, DDR1, DDR2, DIS3, DNMT3A, DOT1L, EED, EGFR, EMSY (C11orf30), EP300, EPHA3, EPHB1, EPHB4, ERBB2, ERBB3, ERBB4, ERCC4, ERG, ERRFI1, ESR1, ETV4, ETV5, ETV6, EWSR1, EZH2, EZR, FAM46C, FANCA, FANCC, FANCG, FANCL, FAS, FBXW7, FGF10, FGF12, FGF14, FGF19, FGF23, FGF3, FGF4, FGF6, FGFR1, FGFR2, FGFR3, FGFR4, FH, FLCN, FLT1, FLT3, FOXL2, FUBP1, GABRA6, GATA3, GATA4, GATA6, GID4 (C17orf39), GNA11, GNA13, GNAQ, GNAS, GRM3, GSK3B, H3F3A, HDAC1, HGF, HNF1A, HRAS, HSD3B1, ID3, IDH1, IDH2, IGF1R, IKBKE, IKZF1, INPP4B, IRF2, IRF4, IRS2, JAK1, JAK2, JAK3, JUN, KDM5A, KDM5C, KDM6A, KDR, KEAP1, KEL, KIT, KLHL6, KMT2A (MLL), KMT2D (MLL2), KRAS, LTK, LYN, MAF, MAP2K1, MAP2K2, MAP2K4, MAP3K1, MAP3K13, MAPK1, MCL1, MDM2, MDM4, MED12, MEF2B, MEN1, MERTK, MET, MITF, MKNK1, MLH1, MPL, MRE11A, MSH2, MSH3, MSH6, MST1R, MTAP, MTOR, MUTYH, MYB, MYC, MYCL, MYCN, MYD88, NBN, NF1, NF2, NFE2L2, NFKB1A, NKX2-1, NOTCH1, NOTCH2, NOTCH3, NPM1, NRAS, NT5C2, NTRK1, NTRK2, NTRK3, NUTM1, P2RY8, PALB2, PARK2, PARP1, PARP2, PARP3, PAX5, PBRM1, PDCD1, PDCD1LG2, PDGFRA, PDGFRB, PDK1, PIK3C2B, PIK3C2G, PIK3CA, PIK3CB, PIK3R1, PIM1, PMS2, POLD1, POLE, PPARG, PPP2R1A, PPP2R2A, PRDM1, PRKAR1A, PRKC1, PTCH1, PTEN, PTPN11, PTPRO, QKI, RAC1, RAD21, RAD51, RAD51B, RAD51C, RAD51D, RAD52, RAD54L, RAF1, RARA, RB1, RBM10, REL, RET, RICTOR, RNF43, ROS1, RPTOR, RSPO2, SDC4, SDHA, SDHB, SDHC, SDHD, SETD2, SF3B1, SGK1, SLC34A2, SMAD2, SMAD4, SMARCA4, SMARCB1, SMO, SNCAIP, SOCS1, SOX2, SOX9, SPEN, SPOP, SRC, STAG2, STAT3, STK11, SUFU, SYK, TBX3, TEK, TERC, TERT, TET2, TGFBR2, TIPARP, TMPRSS2, TNFAIP3, TNFRSF14, TP53, TSC1, TSC2, TYRO3, U2AF1, VEGFA, VHL, WHSC1, WHSC1L1, WT1, XPO1, XRCC2, ZNF217, or ZNF703 gene locus, or any combination thereof.

In some instances, the panel of genomic loci (comprising copy number variants) for which distances are determined in any of the disclosed methods may comprise genomic loci located in the ABL, ALK, ALL, B4GALNT1, BAFF, BCL2, BRAF, BRCA, BTK, CD19, CD20, CD3, CD30, CD319, CD38, CD52, CDK4, CDK6, CML, CRACC, CS1, CTLA-4, dMMR, EGFR, ERBB1, ERBB2, FGFR1-3, FLT3, GD2, HDAC, HER1, HER2, HR, IDH2, IL-1β, IL-6, IL-6R, JAK1, JAK2, JAK3, KIT, KRAS, MEK, MET, MSI-H, mTOR, PARP, PD-1, PDGFR, PDGFRα, PDGFRB, PD-L1, PI3Kδ, PIGF, PTCH, RAF, RANKL, RET, ROS1, SLAMF7, VEGF, VEGFA, or VEGFB gene locus, or any combination thereof.

### Methods of Use

In some instances, the disclosed methods may further comprise one or more of the steps of: (i) obtaining the sample from the subject (e.g., a subject suspected of having or determined to have cancer), (ii) extracting nucleic acid molecules (e.g., a mixture of tumor nucleic acid molecules and non-tumor nucleic acid molecules) from the sample, (iii) ligating one or more adapters to the nucleic acid molecules extracted from the sample (e.g., one or more amplification primers, flow cell adaptor sequences, substrate adapter sequences, or sample index sequences), (iv) performing a methylation conversion reaction to convert, e.g., non-methylated cytosine to uracil, (v) amplifying the nucleic acid molecules (e.g., using a polymerase chain reaction (PCR) amplification technique, a non-PCR amplification technique, or an isothermal amplification technique), (vi) capturing nucleic acid molecules from the amplified nucleic acid molecules (e.g., by hybridization to one or more bait molecules, where the bait molecules each comprise one or more nucleic acid molecules that each comprising a region that is complementary to a region of a captured nucleic acid molecule), (vii) sequencing the nucleic acid molecules extracted from the sample (or library proxies derived therefrom) using, e.g., a next-generation (massively parallel) sequencing technique, a whole genome sequencing (WGS) technique, a whole exome sequencing technique, a targeted sequencing technique, a direct sequencing technique, or a Sanger sequencing technique) using, e.g., a next-generation (massively parallel) sequencer, (viii) combining the nucleic acid sequence data (including, e.g., variant data, copy number data, methylation status data, etc., of the sequenced nucleic acid molecules) with other biomarker data modalities including, but not limited to, proteomics-based biomarker data (e.g., the detection of specific polypeptides, such as proteins) or fragmentomics-based biomarker data (e.g., the detection of certain attributes related to nucleic acid fragments, such as fragment size or the sequences of fragment ends), to determine, for example, the presence of ctDNA in the sample and/or to determine a diagnostic, prognostic, and/or treatment response prediction for the subject, and (ix) generating, displaying, transmitting, and/or delivering a report (e.g., an electronic, web-based, or paper report) to the subject (or patient), a caregiver, a healthcare provider, a physician, an oncologist, an electronic medical record system, a hospital, a clinic, a third-party payer, an insurance company, or a government office. In some instances, the report comprises output from the methods described herein. In some instances, all or a portion of the report may be displayed in the graphical user interface of an online or web-based healthcare portal. In some instances, the report is transmitted via a computer network or peer-to-peer connection.

The disclosed methods may be used with any of a variety of samples. For example, in some instances, the sample may comprise a tissue biopsy sample, a liquid biopsy sample, or a normal control. In some instances, the sample may be a liquid biopsy sample and may comprise blood, plasma, cerebrospinal fluid, sputum, stool, urine, or saliva. In some instances, the sample may be a liquid biopsy sample and may comprise circulating tumor cells (CTCs). In some instances, the sample may be a liquid biopsy sample and may comprise cell-free DNA (cfDNA). In some instances, the cell-free DNA (cfDNA), or a portion thereof, may comprise circulating tumor DNA (ctDNA). In some instances, the liquid biopsy sample may comprise a combination of cell-free DNA (cfDNA) and circulating tumor DNA (ctDNA).

In some instances, the nucleic acid molecules extracted from a sample may comprise a mixture of tumor nucleic acid molecules and non-tumor nucleic acid molecules. In some instances, the tumor nucleic acid molecules may be derived from a tumor portion of a heterogeneous tissue biopsy sample, and the non-tumor nucleic acid molecules may be derived from a normal portion of the heterogeneous tissue biopsy sample. In some instances, the sample may comprise a liquid biopsy sample, and the tumor nucleic acid molecules may be derived from a circulating tumor DNA (ctDNA) fraction of the liquid biopsy sample while the non-tumor nucleic acid molecules may be derived from a non-tumor, cell-free DNA (cfDNA) fraction of the liquid biopsy sample.

In some instances, the disclosed methods for detecting HJBs may be used to diagnose (or as part of a diagnosis of) the presence of disease or other condition (e.g., cancer, genetic disorders (such as Down Syndrome and Fragile X), neurological disorders, or any other disease type where detection of variants, e.g., copy number alternations, are relevant to diagnosing, treating, or predicting said disease) in a subject (e.g., a patient). In some instances, the disclosed methods may be applicable to diagnosis of any of a variety of cancers as described elsewhere herein.

In some instances, the disclosed methods for detecting HJBs may be used to predict genetic disorders in fetal DNA (e.g., for invasive or non-invasive prenatal testing). For example, sequence read data obtained by sequencing fetal DNA extracted from samples obtained using invasive amniocentesis, chorionic villus sampling (cVS), or fetal umbilical cord sampling techniques, or obtained using non-invasive sampling of cell-free DNA (cfDNA) samples (which comprises a mix of maternal cfDNA and fetal cfDNA), may be processed according to the disclosed methods to identify variants, e.g., copy number alterations, associated with, e.g., Down Syndrome (trisomy 21), trisomy 18, trisomy 13, and extra or missing copies of the X and Y chromosomes.

In some instances, the disclosed methods for detecting HJBs may be used to select a subject (e.g., a patient) for a clinical trial based on the presence or absence of HJBs. In some instances, patient selection for clinical trials based on, e.g., the presence or absence of HJBs, may accelerate the development of targeted therapies and improve the healthcare outcomes for treatment decisions.

In some instances, the disclosed methods for detecting HJBs may be used to select an appropriate therapy or treatment (e.g., an anti-cancer therapy or anti-cancer treatment) for a subject. In some instances, for example, the anti-cancer therapy or treatment may comprise use of a poly (ADP-ribose) polymerase inhibitor (PARPi), a platinum compound, chemotherapy, radiation therapy, a targeted therapy, an immunotherapy, a neoantigen-based therapy, surgery, or any combination thereof.

In some instances, the anti-cancer therapy or treatment may comprise a targeted anti-cancer therapy or treatment (e.g., a monoclonal antibody-based therapy, an enzyme inhibitor-based therapy, an antibody-drug conjugate therapy, a hormone therapy, and/or a targeted radiotherapy) that targets specific molecules required for cancer cell growth, division, and spreading. In some instances, the targeted anti-cancer therapy or treatment may comprise abemaciclib (Verzenio), abiraterone acetate (Zytiga), acalabrutinib (Calquence), ado-trastuzumab emtansine (Kadcyla), afatinib dimaleate (Gilotrif), alectinib (Alecensa), alemtuzumab (Campath), alitretinoin (Panretin), alpelisib (Piqray), amivantamab-vmjw (Rybrevant), anastrozole (Arimidex), apalutamide (Erleada), asciminib hydrochloride (Scemblix), atezolizumab (Tecentriq), avapritinib (Ayvakit), avelumab (Bavencio), axicabtagene ciloleucel (Yescarta), axitinib (Inlyta), belantamab mafodotin-blmf (Blenrep), belimumab (Benlysta), belinostat (Beleodaq), belzutifan (Welireg), bevacizumab (Avastin), bexarotene (Targretin), binimetinib (Mektovi), blinatumomab (Blincyto), bortezomib (Velcade), bosutinib (Bosulif), brentuximab vedotin (Adcetris), brexucabtagene autoleucel (Tecartus), brigatinib (Alunbrig), cabazitaxel (Jevtana), cabozantinib (Cabometyx), cabozantinib (Cabometyx, Cometriq), canakinumab (Ilaris), capmatinib hydrochloride (Tabrecta), carfilzomib (Kyprolis), cemiplimab-rwlc (Libtayo), ceritinib (LDK378/Zykadia), cetuximab (Erbitux), cobimetinib (Cotellic), crizotinib (Xalkori), dabrafenib (Tafinlar), dacomitinib (Vizimpro), daratumumab (Darzalex), daratumumab and hyaluronidase-fihj (Darzalex Faspro), darolutamide (Nubeqa), dasatinib (Sprycel), denileukin diftitox (Ontak), denosumab (Xgeva), dinutuximab (Unituxin), dostarlimab-gxly (Jemperli), durvalumab (Imfinzi), duvelisib (Copiktra), elotuzumab (Empliciti), enasidenib mesylate (Idhifa), encorafenib (Braftovi), enfortumab vedotin-ejfv (Padcev), entrectinib (Rozlytrek), enzalutamide (Xtandi), erdafitinib (Balversa), erlotinib (Tarceva), everolimus (Afinitor), exemestane (Aromasin), fam-trastuzumab deruxtecan-nxki (Enhertu), fedratinib hydrochloride (Inrebic), fulvestrant (Faslodex), gefitinib (Iressa), gemtuzumab ozogamicin (Mylotarg), gilteritinib (Xospata), glasdegib maleate (Daurismo), hyaluronidase-zzxf (Phesgo), ibrutinib (Imbruvica), ibritumomab tiuxetan (Zevalin), idecabtagene vicleucel (Abecma), idelalisib (Zydelig), imatinib mesylate (Gleevec), infigratinib phosphate (Truseltiq), inotuzumab ozogamicin (Besponsa), ipilimumab (Yervoy), isatuximab-irfc (Sarclisa), ivosidenib (Tibsovo), ixazomib citrate (Ninlaro), lanreotide acetate (Somatuline Depot), lapatinib (Tykerb), larotrectinib sulfate (Vitrakvi), lenvatinib mesylate (Lenvima), letrozole (Femara), lisocabtagene maraleucel (Breyanzi), loncastuximab tesirine-lpyl (Zynlonta), lorlatinib (Lorbrena), lutetium Lu 177-dotatate (Lutathera), margetuximab-cmkb (Margenza), midostaurin (Rydapt), mobocertinib succinate (Exkivity), mogamulizumab-kpkc (Poteligeo), moxetumomab pasudotox-tdfk (Lumoxiti), naxitamab-gqgk (Danyelza), necitumumab (Portrazza), neratinib maleate (Nerlynx), nilotinib (Tasigna), niraparib tosylate monohydrate (Zejula), nivolumab (Opdivo), obinutuzumab (Gazyva), ofatumumab (Arzerra), olaparib (Lynparza), olaratumab (Lartruvo), osimertinib (Tagrisso), palbociclib (Ibrance), panitumumab (Vectibix), pazopanib (Votrient), pembrolizumab (Keytruda), pemigatinib (Pemazyre), pertuzumab (Perjeta), pexidartinib hydrochloride (Turalio), polatuzumab vedotin-piiq (Polivy), ponatinib hydrochloride (Iclusig), pralatrexate (Folotyn), pralsetinib (Gavreto), radium 223 dichloride (Xofigo), ramucirumab (Cyramza), regorafenib (Stivarga), ribociclib (Kisqali), ripretinib (Qinlock), rituximab (Rituxan), rituximab and hyaluronidase human (Rituxan Hycela), romidepsin (Istodax), rucaparib camsylate (Rubraca), ruxolitinib phosphate (Jakafi), sacituzumab govitecan-hziy (Trodelvy), seliciclib, selinexor (Xpovio), selpercatinib (Retevmo), selumetinib sulfate (Koselugo), siltuximab (Sylvant), sirolimus protein-bound particles (Fyarro), sonidegib (Odomzo), sorafenib (Nexavar), sotorasib (Lumakras), sunitinib (Sutent), tafasitamab-cxix (Monjuvi), tagraxofusp-erzs (Elzonris), talazoparib tosylate (Talzenna), tamoxifen (Nolvadex), tazemetostat hydrobromide (Tazverik), tebentafusp-tebn (Kimmtrak), temsirolimus (Torisel), tepotinib hydrochloride (Tepmetko), tisagenlecleucel (Kymriah), tisotumab vedotin-tftv (Tivdak), tocilizumab (Actemra), tofacitinib (Xeljanz), tositumomab (Bexxar), trametinib (Mekinist), trastuzumab (Herceptin), tretinoin (Vesanoid), tivozanib hydrochloride (Fotivda), toremifene (Fareston), tucatinib (Tukysa), umbralisib tosylate (Ukoniq), vandetanib (Caprelsa), vemurafenib (Zelboraf), venetoclax (Venclexta), vismodegib (Erivedge), vorinostat (Zolinza), zanubrutinib (Brukinsa), ziv-aflibercept (Zaltrap), or any combination thereof.

In some instances, the anti-cancer therapy or treatment may comprise an immunotherapy (e.g., a cancer treatment that acts by stimulating the immune system to fight cancer). In some instances, the immunotherapy can be, for example, an immune system modulator (e.g., a cytokine, such as an interferon or interleukin), an immune checkpoint inhibitor (such as an anti-PD-1 or anti-PD-L1 antibody), a T-cell transfer therapy (e.g., a tumor infiltrating lymphocyte (TIL) therapy in lymphocytes extracted from a patient's tumor are selected for their ability to recognize tumor cells and propagated prior to reintroduction into the patient, or a CAR T-cell therapy in which a patient's T-cells are modified to express the CAR protein prior to reintroduction into the patient), a monoclonal antibody-based therapy (e.g., a monoclonal antibody that binds to cell surface markers on cancer cells to facilitate recognition by the immune system), or a cancer treatment vaccine (e.g., a vaccine based on tumor cells, tumor-associated neoantigens, or dendritic cells, etc., that stimulates the immune system to fight cancer).

In some instances, the anti-cancer therapy or treatment may comprise a neoantigen-based therapy. Non-limiting examples of neoantigen-based therapies include T-cell receptor (TCR) engineered T-cell (TCR-T) therapies, chimeric antigen receptor T-cell (CAR-T) therapies, TCR bispecific antibody therapies, and cancer vaccines. TCR-T therapies are produced by genetically engineering a patient's T-cells to express T-cell receptors that are specific to neoantigens of interest, and then infusing them back into the patient. CAR-T therapies are produced by genetically engineering a patient's T-cells to express chimeric antigen receptor molecules which contain an intracellular signaling and co-signaling domain as well as an extracellular antigen-binding domain; CAR-T therapies don't always rely on neoantigen presentation, but can be designed to be directed towards neoantigens. TCR bispecific antibody therapies are small, engineered antibody molecules that comprise a neoantigen-specific TCR on one end and a CD3-directed single-chain variable fragment on the other end. Cancer vaccines can include RNA molecules, DNA molecules, peptides, or a combination thereof that are designed to boost the immune system's ability to find and destroy neoantigen-presenting cells.

In some instances, the disclosed methods for detecting HJBs may be used in treating a disease (e.g., a cancer) in a subject. For example, in response to detecting HJBs using any of the methods disclosed herein, an effective amount of an anti-cancer therapy or anti-cancer treatment may be administered to the subject.

In some instances, the disclosed methods for detecting HJBs may be used for monitoring disease progression or recurrence (e.g., cancer or tumor progression or recurrence) in a subject. For example, in some instances, the methods may be used to determine a correlation between copy number at one or more genomic loci and distance for each of the one or more genomic loci from a centromere of a chromosome in a first sample obtained from the subject at a first time point, and used to determine a correlation between copy number at one or more genomic loci and distance for each of the one or more genomic loci from a centromere of a chromosome in a second sample obtained from the subject at a second time point, where comparison of the first determination of a correlation between copy number at one or more genomic loci and distance for each of the one or more genomic loci from a centromere of a chromosome and the second determination of a correlation between copy number at one or more genomic loci and distance for each of the one or more genomic loci from a centromere of a chromosome allows one to monitor disease progression or recurrence. In some instances, the first time point is chosen before the subject has been administered a therapy or treatment, and the second time point is chosen after the subject has been administered the therapy or treatment.

In some instances, the disclosed methods may be used for adjusting a therapy or treatment (e.g., an anti-cancer treatment or anti-cancer therapy) for a subject, e.g., by adjusting a treatment dose and/or selecting a different treatment in response to a change in the determination of a correlation between copy number at one or more genomic loci and distance for each of the one or more genomic loci from a centromere of a chromosome.

In some instances, the value of a correlation between copy number at one or more genomic loci and distance for each of the one or more genomic loci from a centromere of a chromosome determined using the disclosed methods may be used as a prognostic or diagnostic indicator associated with the sample. For example, in some instances, the prognostic or diagnostic indicator may comprise an indicator of the presence of a disease (e.g., cancer) in the sample, an indicator of the probability that a disease (e.g., cancer) is present in the sample, an indicator of the probability that the subject from which the sample was derived will develop a disease (e.g., cancer) (i.e., a risk factor), or an indicator of the likelihood that the subject from which the sample was derived will respond to a particular therapy or treatment.

In some instances, the disclosed methods for detecting HJBs may be implemented as part of a genomic profiling process that comprises identification of the presence of variant sequences at one or more gene loci in a sample derived from a subject as part of detecting, monitoring, predicting a risk factor, or selecting a treatment for a particular disease, e.g., cancer. In some instances, the variant panel selected for genomic profiling may comprise the detection of variant sequences at a selected set of gene loci. In some instances, the variant panel selected for genomic profiling may comprise detection of variant sequences at a number of gene loci through comprehensive genomic profiling (CGP), which is a next-generation sequencing (NGS) approach used to assess hundreds of genes (including relevant cancer biomarkers) in a single assay. Inclusion of the disclosed methods for detecting HJBs as part of a genomic profiling process (or inclusion of the output from the disclosed methods for detecting HJBs as part of the genomic profile of the subject) can improve the validity of, e.g., disease detection calls and treatment decisions, made on the basis of the genomic profile by, for example, independently confirming the presence of HJBs in a given patient sample.

In some instances, a genomic profile may comprise information on the presence of genes (or variant sequences thereof), copy number variations, epigenetic traits, proteins (or modifications thereof), and/or other biomarkers in an individual's genome and/or proteome, as well as information on the individual's corresponding phenotypic traits and the interaction between genetic or genomic traits, phenotypic traits, and environmental factors.

In some instances, a genomic profile for the subject may comprise results from a comprehensive genomic profiling (CGP) test, a nucleic acid sequencing-based test, a gene expression profiling test, a cancer hotspot panel test, a DNA methylation test, a DNA fragmentation test, an RNA fragmentation test, or any combination thereof.

In some instances, the method can further include administering or applying a treatment or therapy (e.g., an anti-cancer agent, anti-cancer treatment, or anti-cancer therapy) to the subject based on the generated genomic profile. An anti-cancer agent or anti-cancer treatment may refer to a compound that is effective in the treatment of cancer cells. Examples of anti-cancer agents or anti-cancer therapies include, but not limited to, alkylating agents, antimetabolites, natural products, hormones, chemotherapy, radiation therapy, immunotherapy, surgery, or a therapy configured to target a defect in a specific cell signaling pathway, e.g., a defect in a DNA mismatch repair (MMR) pathway.

### Samples

The disclosed methods and systems may be used with any of a variety of samples (also referred to herein as specimens) comprising nucleic acids (e.g., DNA or RNA) that are collected from a subject (e.g., a patient). Examples of a sample include, but are not limited to, a tumor sample, a tissue sample, a biopsy sample (e.g., a tissue biopsy, a liquid biopsy, or both), a blood sample (e.g., a peripheral whole blood sample), a blood plasma sample, a blood serum sample, a lymph sample, a saliva sample, a sputum sample, a urine sample, a gynecological fluid sample, a circulating tumor cell (CTC) sample, a cerebral spinal fluid (CSF) sample, a pericardial fluid sample, a pleural fluid sample, an ascites (peritoneal fluid) sample, a feces (or stool) sample, or other body fluid, secretion, and/or excretion sample (or cell sample derived therefrom). In certain instances, the sample may be frozen sample or a formalin-fixed paraffin-embedded (FFPE) sample.

In some instances, the sample may be collected by tissue resection (e.g., surgical resection), needle biopsy, bone marrow biopsy, bone marrow aspiration, skin biopsy, endoscopic biopsy, fine needle aspiration, oral swab, nasal swab, vaginal swab or a cytology smear, scrapings, washings or lavages (such as a ductal lavage or bronchoalveolar lavage), etc.

In some instances, the sample is a liquid biopsy sample, and may comprise, e.g., whole blood, blood plasma, blood serum, urine, stool, sputum, saliva, or cerebrospinal fluid. In some instances, the sample may be a liquid biopsy sample and may comprise circulating tumor cells (CTCs). In some instances, the sample may be a liquid biopsy sample and may comprise cell-free DNA (cfDNA), circulating tumor DNA (ctDNA), or any combination thereof.

In some instances, the sample may comprise one or more premalignant or malignant cells. Premalignant, as used herein, refers to a cell or tissue that is not yet malignant but is poised to become malignant. In certain instances, the sample may be acquired from a solid tumor, a soft tissue tumor, or a metastatic lesion. In certain instances, the sample may be acquired from a hematologic malignancy or pre-malignancy. In other instances, the sample may comprise a tissue or cells from a surgical margin. In certain instances, the sample may comprise tumor-infiltrating lymphocytes. In some instances, the sample may comprise one or more non-malignant cells. In some instances, the sample may be, or is part of, a primary tumor or a metastasis (e.g., a metastasis biopsy sample). In some instances, the sample may be obtained from a site (e.g., a tumor site) with the highest percentage of tumor (e.g., tumor cells) as compared to adjacent sites (e.g., sites adjacent to the tumor). In some instances, the sample may be obtained from a site (e.g., a tumor site) with the largest tumor focus (e.g., the largest number of tumor cells as visualized under a microscope) as compared to adjacent sites (e.g., sites adjacent to the tumor).

In some instances, the disclosed methods may further comprise analyzing a primary control (e.g., a normal tissue sample). In some instances, the disclosed methods may further comprise determining if a primary control is available and, if so, isolating a control nucleic acid (e.g., DNA) from said primary control. In some instances, the sample may comprise any normal control (e.g., a normal adjacent tissue (NAT)) if no primary control is available. In some instances, the sample may be or may comprise histologically normal tissue. In some instances, the method includes evaluating a sample, e.g., a histologically normal sample (e.g., from a surgical tissue margin) using the methods described herein. In some instances, the disclosed methods may further comprise acquiring a sub-sample enriched for non-tumor cells, e.g., by macro-dissecting non-tumor tissue from said NAT in a sample not accompanied by a primary control. In some instances, the disclosed methods may further comprise determining that no primary control and no NAT is available, and marking said sample for analysis without a matched control.

In some instances, samples obtained from histologically normal tissues (e.g., otherwise histologically normal surgical tissue margins) may still comprise a genetic alteration such as a variant sequence as described herein. The methods may thus further comprise re-classifying a sample based on the presence of the detected genetic alteration. In some instances, multiple samples (e.g., from different subjects) are processed simultaneously.

The disclosed methods and systems may be applied to the analysis of nucleic acids extracted from any of variety of tissue samples (or disease states thereof), e.g., solid tissue samples, soft tissue samples, metastatic lesions, or liquid biopsy samples. Examples of tissues include, but are not limited to, connective tissue, muscle tissue, nervous tissue, epithelial tissue, and blood. Tissue samples may be collected from any of the organs within an animal or human body. Examples of human organs include, but are not limited to, the brain, heart, lungs, liver, kidneys, pancreas, spleen, thyroid, mammary glands, uterus, prostate, large intestine, small intestine, bladder, bone, skin, etc.

In some instances, the nucleic acids extracted from the sample may comprise deoxyribonucleic acid (DNA) molecules. Examples of DNA that may be suitable for analysis by the disclosed methods include, but are not limited to, genomic DNA or fragments thereof, mitochondrial DNA or fragments thereof, cell-free DNA (cfDNA), and circulating tumor DNA (ctDNA). Cell-free DNA (cfDNA) is comprised of fragments of DNA that are released from normal and/or cancerous cells during apoptosis and necrosis, and circulate in the blood stream and/or accumulate in other bodily fluids. Circulating tumor DNA (ctDNA) is comprised of fragments of DNA that are released from cancerous cells and tumors that circulate in the blood stream and/or accumulate in other bodily fluids.

In some instances, DNA is extracted from nucleated cells from the sample. In some instances, a sample may have a low nucleated cellularity, e.g., when the sample is comprised mainly of erythrocytes, lesional cells that contain excessive cytoplasm, or tissue with fibrosis. In some instances, a sample with low nucleated cellularity may require more, e.g., greater, tissue volume for DNA extraction.

In some instances, the nucleic acids extracted from the sample may comprise ribonucleic acid (RNA) molecules. Examples of RNA that may be suitable for analysis by the disclosed methods include, but are not limited to, total cellular RNA, total cellular RNA after depletion of certain abundant RNA sequences (e.g., ribosomal RNAs), cell-free RNA (cfRNA), messenger RNA (mRNA) or fragments thereof, the poly(A)-tailed mRNA fraction of the total RNA, ribosomal RNA (rRNA) or fragments thereof, transfer RNA (tRNA) or fragments thereof, and mitochondrial RNA or fragments thereof. In some instances, RNA may be extracted from the sample and converted to complementary DNA (cDNA) using, e.g., a reverse transcription reaction. In some instances, the cDNA is produced by random-primed cDNA synthesis methods. In other instances, the cDNA synthesis is initiated at the poly(A) tail of mature mRNAs by priming with oligo(dT)-containing oligonucleotides. Methods for depletion, poly(A) enrichment, and cDNA synthesis are well known to those of skill in the art.

In some instances, the sample may comprise a tumor content (e.g., comprising tumor cells or tumor cell nuclei), or a non-tumor content (e.g., immune cells, fibroblasts, and other non-tumor cells). In some instances, the tumor content of the sample may constitute a sample metric. In some instances, the sample may comprise a tumor content of at least 5-50%, 10-40%, 15-25%, or 20-30% tumor cell nuclei. In some instances, the sample may comprise a tumor content of at least 5%, at least 10%, at least 20%, at least 30%, at least 40%, or at least 50% tumor cell nuclei. In some instances, the percent tumor cell nuclei (e.g., sample fraction) is determined (e.g., calculated) by dividing the number of tumor cells in the sample by the total number of all cells within the sample that have nuclei. In some instances, for example when the sample is a liver sample comprising hepatocytes, a different tumor content calculation may be required due to the presence of hepatocytes having nuclei with twice, or more than twice, the DNA content of other, e.g., non-hepatocyte, somatic cell nuclei. In some instances, the sensitivity of detection of a genetic alteration, e.g., a variant sequence, or a determination of, e.g., microsatellite instability, may depend on the tumor content of the sample. For example, a sample having a lower tumor content can result in lower sensitivity of detection for a given size sample.

In some instances, as noted above, the sample comprises nucleic acid (e.g., DNA, RNA (or a cDNA derived from the RNA), or both), e.g., from a tumor or from normal tissue. In certain instances, the sample may further comprise a non-nucleic acid component, e.g., cells, protein, carbohydrate, or lipid, e.g., from the tumor or normal tissue.

### Subjects

In some instances, the sample is obtained (e.g., collected) from a subject (e.g., patient) with a condition or disease (e.g., a hyperproliferative disease or a non-cancer indication) or suspected of having the condition or disease. In some instances, the hyperproliferative disease is a cancer. In some instances, the cancer is a solid tumor or a metastatic form thereof. In some instances, the cancer is a hematological cancer, e.g., a leukemia or lymphoma.

In some instances, the subject has a cancer or is at risk of having a cancer. For example, in some instances, the subject has a genetic predisposition to a cancer (e.g., having a genetic mutation that increases his or her baseline risk for developing a cancer). In some instances, the subject has been exposed to an environmental perturbation (e.g., radiation or a chemical) that increases his or her risk for developing a cancer. In some instances, the subject is in need of being monitored for development of a cancer. In some instances, the subject is in need of being monitored for cancer progression or regression, e.g., after being treated with an anti-cancer therapy (or anti-cancer treatment). In some instances, the subject is in need of being monitored for relapse of cancer. In some instances, the subject is in need of being monitored for minimum residual disease (MRD). In some instances, the subject has been, or is being treated, for cancer. In some instances, the subject has not been treated with an anti-cancer therapy (or anti-cancer treatment).

In some instances, the subject (e.g., a patient) is being treated, or has been previously treated, with one or more targeted therapies. In some instances, e.g., for a patient who has been previously treated with a targeted therapy, a post-targeted therapy sample (e.g., specimen) is obtained (e.g., collected). In some instances, the post-targeted therapy sample is a sample obtained after the completion of the targeted therapy.

In some instances, the patient has not been previously treated with a targeted therapy. In some instances, e.g., for a patient who has not been previously treated with a targeted therapy, the sample comprises a resection, e.g., an original resection, or a resection following recurrence (e.g., following a disease recurrence post-therapy).

### Cancers

In some instances, the sample is acquired from a subject having a cancer. Exemplary cancers include, but are not limited to, B cell cancer (e.g., multiple myeloma), melanomas, breast cancer, lung cancer (such as non-small cell lung carcinoma or NSCLC), bronchus cancer, colorectal cancer, prostate cancer, pancreatic cancer, stomach cancer, ovarian cancer, urinary bladder cancer, brain or central nervous system cancer, peripheral nervous system cancer, esophageal cancer, cervical cancer, uterine or endometrial cancer, cancer of the oral cavity or pharynx, liver cancer, kidney cancer, testicular cancer, biliary tract cancer, small bowel or appendix cancer, salivary gland cancer, thyroid gland cancer, adrenal gland cancer, osteosarcoma, chondrosarcoma, cancer of hematological tissues, adenocarcinomas, inflammatory myofibroblastic tumors, gastrointestinal stromal tumor (GIST), colon cancer, multiple myeloma (MM), myelodysplastic syndrome (MDS), myeloproliferative disorder (MPD), acute lymphocytic leukemia (ALL), acute myelocytic leukemia (AML), chronic myelocytic leukemia (CML), chronic lymphocytic leukemia (CLL), polycythemia Vera, Hodgkin lymphoma, non-Hodgkin lymphoma (NHL), soft-tissue sarcoma, fibrosarcoma, myxosarcoma, liposarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, squamous cell carcinoma, basal cell carcinoma, adenocarcinoma, sweat gland carcinoma, sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, seminoma, embryonal carcinoma, Wilms' tumor, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, neuroblastoma, retinoblastoma, follicular lymphoma, diffuse large B-cell lymphoma, mantle cell lymphoma, hepatocellular carcinoma, thyroid cancer, gastric cancer, head and neck cancer, small cell cancers, essential thrombocythemia, agnogenic myeloid metaplasia, hypereosinophilic syndrome, systemic mastocytosis, familiar hypereosinophilia, chronic eosinophilic leukemia, neuroendocrine cancers, carcinoid tumors, and the like.

In some instances, the cancer comprises acute lymphoblastic leukemia (Philadelphia chromosome positive), acute lymphoblastic leukemia (precursor B-cell), acute myeloid leukemia (FLT3+), acute myeloid leukemia (with an IDH2 mutation), anaplastic large cell lymphoma, basal cell carcinoma, B-cell chronic lymphocytic leukemia, bladder cancer, breast cancer (HER2 overexpressed/amplified), breast cancer (HER2+), breast cancer (HR+, HER2−), cervical cancer, cholangiocarcinoma, chronic lymphocytic leukemia, chronic lymphocytic leukemia (with 17p deletion), chronic myelogenous leukemia, chronic myelogenous leukemia (Philadelphia chromosome positive), classical Hodgkin lymphoma, colorectal cancer, colorectal cancer (dMMR and MSI-H), colorectal cancer (KRAS wild type), cryopyrin-associated periodic syndrome, a cutaneous T-cell lymphoma, dermatofibrosarcoma protuberans, a diffuse large B-cell lymphoma, fallopian tube cancer, a follicular B-cell non-Hodgkin lymphoma, a follicular lymphoma, gastric cancer, gastric cancer (HER2+), a gastroesophageal junction (GEJ) adenocarcinoma, a gastrointestinal stromal tumor, a gastrointestinal stromal tumor (KIT+), a giant cell tumor of the bone, a glioblastoma, granulomatosis with polyangiitis, a head and neck squamous cell carcinoma, a hepatocellular carcinoma, Hodgkin lymphoma, juvenile idiopathic arthritis, lupus erythematosus, a mantle cell lymphoma, medullary thyroid cancer, melanoma, a melanoma with a BRAF V600 mutation, a melanoma with a BRAF V600E or V600K mutation, Merkel cell carcinoma, multicentric Castleman's disease, multiple hematologic malignancies including Philadelphia chromosome-positive ALL and CML, multiple myeloma, myelofibrosis, a non-Hodgkin's lymphoma, a nonresectable subependymal giant cell astrocytoma associated with tuberous sclerosis, a non-small cell lung cancer, a non-small cell lung cancer (ALK+), a non-small cell lung cancer (PD-L1+), a non-small cell lung cancer (with ALK fusion or ROS1 gene alteration), a non-small cell lung cancer (with BRAF V600E mutation), a non-small cell lung cancer (with an EGFR exon 19 deletion or exon 21 substitution (L858R) mutations), a non-small cell lung cancer (with an EGFR T790M mutation), ovarian cancer, ovarian cancer (with a BRCA mutation), pancreatic cancer, a pancreatic, gastrointestinal, or lung origin neuroendocrine tumor, a pediatric neuroblastoma, a peripheral T-cell lymphoma, peritoneal cancer, prostate cancer, a renal cell carcinoma, rheumatoid arthritis, a small lymphocytic lymphoma, a soft tissue sarcoma, a solid tumor (MSI-H/dMMR), a squamous cell cancer of the head and neck, a squamous non-small cell lung cancer, thyroid cancer, a thyroid carcinoma, urothelial cancer, a urothelial carcinoma, or Waldenstrom's macroglobulinemia.

In some instances, the cancer is a hematologic malignancy (or premaligancy). As used herein, a hematologic malignancy refers to a tumor of the hematopoietic or lymphoid tissues, e.g., a tumor that affects blood, bone marrow, or lymph nodes. Exemplary hematologic malignancies include, but are not limited to, leukemia (e.g., acute lymphoblastic leukemia (ALL), acute myeloid leukemia (AML), chronic lymphocytic leukemia (CLL), chronic myelogenous leukemia (CML), hairy cell leukemia, acute monocytic leukemia (AMOL), chronic myelomonocytic leukemia (CMML), juvenile myelomonocytic leukemia (JMML), or large granular lymphocytic leukemia), lymphoma (e.g., AIDS-related lymphoma, cutaneous T-cell lymphoma, Hodgkin lymphoma (e.g., classical Hodgkin lymphoma or nodular lymphocyte-predominant Hodgkin lymphoma), mycosis fungoides, non-Hodgkin lymphoma (e.g., B-cell non-Hodgkin lymphoma (e.g., Burkitt lymphoma, small lymphocytic lymphoma (CLL/SLL), diffuse large B-cell lymphoma, follicular lymphoma, immunoblastic large cell lymphoma, precursor B-lymphoblastic lymphoma, or mantle cell lymphoma) or T-cell non-Hodgkin lymphoma (mycosis fungoides, anaplastic large cell lymphoma, or precursor T-lymphoblastic lymphoma)), primary central nervous system lymphoma, Sézary syndrome, Waldenström macroglobulinemia), chronic myeloproliferative neoplasm, Langerhans cell histiocytosis, multiple myeloma/plasma cell neoplasm, myelodysplastic syndrome, or myelodysplastic/myeloproliferative neoplasm.

### Nucleic Acid Extraction and Processing

DNA or RNA may be extracted from tissue samples, biopsy samples, blood samples, or other bodily fluid samples using any of a variety of techniques known to those of skill in the art (see, e.g., Example 1 of International Patent Application Publication No. WO 2012/092426; Tan, et al. (2009), “DNA, RNA, and Protein Extraction: The Past and The Present”, J. Biomed. Biotech. 2009:574398; the technical literature for the Maxwell® 16 LEV Blood DNA Kit (Promega Corporation, Madison, WI); and the Maxwell 16 Buccal Swab LEV DNA Purification Kit Technical Manual (Promega Literature #TM333, Jan. 1, 2011, Promega Corporation, Madison, WI)). Protocols for RNA isolation are disclosed in, e.g., the Maxwell® 16 Total RNA Purification Kit Technical Bulletin (Promega Literature #TB351, August 2009, Promega Corporation, Madison, WI).

A typical DNA extraction procedure, for example, comprises (i) collection of the fluid sample, cell sample, or tissue sample from which DNA is to be extracted, (ii) disruption of cell membranes (i.e., cell lysis), if necessary, to release DNA and other cytoplasmic components, (iii) treatment of the fluid sample or lysed sample with a concentrated salt solution to precipitate proteins, lipids, and RNA, followed by centrifugation to separate out the precipitated proteins, lipids, and RNA, and (iv) purification of DNA from the supernatant to remove detergents, proteins, salts, or other reagents used during the cell membrane lysis step.

Disruption of cell membranes may be performed using a variety of mechanical shear (e.g., by passing through a French press or fine needle) or ultrasonic disruption techniques. The cell lysis step often comprises the use of detergents and surfactants to solubilize lipids the cellular and nuclear membranes. In some instances, the lysis step may further comprise use of proteases to break down protein, and/or the use of an RNase for digestion of RNA in the sample.

Examples of suitable techniques for DNA purification include, but are not limited to, (i) precipitation in ice-cold ethanol or isopropanol, followed by centrifugation (precipitation of DNA may be enhanced by increasing ionic strength, e.g., by addition of sodium acetate), (ii) phenol-chloroform extraction, followed by centrifugation to separate the aqueous phase containing the nucleic acid from the organic phase containing denatured protein, and (iii) solid phase chromatography where the nucleic acids adsorb to the solid phase (e.g., silica or other) depending on the pH and salt concentration of the buffer.

In some instances, cellular and histone proteins bound to the DNA may be removed either by adding a protease or by having precipitated the proteins with sodium or ammonium acetate, or through extraction with a phenol-chloroform mixture prior to a DNA precipitation step.

In some instances, DNA may be extracted using any of a variety of suitable commercial DNA extraction and purification kits. Examples include, but are not limited to, the QIAamp (for isolation of genomic DNA from human samples) and DNAeasy (for isolation of genomic DNA from animal or plant samples) kits from Qiagen (Germantown, MD) or the Maxwell® and ReliaPrep™ series of kits from Promega (Madison, WI).

As noted above, in some instances the sample may comprise a formalin-fixed (also known as formaldehyde-fixed, or paraformaldehyde-fixed), paraffin-embedded (FFPE) tissue preparation. For example, the FFPE sample may be a tissue sample embedded in a matrix, e.g., an FFPE block. Methods to isolate nucleic acids (e.g., DNA) from formaldehyde- or paraformaldehyde-fixed, paraffin-embedded (FFPE) tissues are disclosed in, e.g., Cronin, et al., (2004) Am J Pathol. 164(1):35-42; Masuda, et al., (1999) Nucleic Acids Res. 27(22):4436-4443; Specht, et al., (2001) Am J Pathol. 158(2):419-429; the Ambion RecoverAll™ Total Nucleic Acid Isolation Protocol (Ambion, Cat. No. AM1975, September 2008); the Maxwell® 16 FFPE Plus LEV DNA Purification Kit Technical Manual (Promega Literature #TM349, February 2011); the E.Z.N.A.® FFPE DNA Kit Handbook (OMEGA bio-tek, Norcross, GA, product numbers D3399-00, D3399-01, and D3399-02, June 2009); and the QIAamp® DNA FFPE Tissue Handbook (Qiagen, Cat. No. 37625, October 2007). For example, the RecoverAll™ Total Nucleic Acid Isolation Kit uses xylene at elevated temperatures to solubilize paraffin-embedded samples and a glass-fiber filter to capture nucleic acids. The Maxwell® 16 FFPE Plus LEV DNA Purification Kit is used with the Maxwell® 16 Instrument for purification of genomic DNA from 1 to 10 μm sections of FFPE tissue. DNA is purified using silica-clad paramagnetic particles (PMPs), and eluted in low elution volume. The E.Z.N.A.® FFPE DNA Kit uses a spin column and buffer system for isolation of genomic DNA. QIAamp® DNA FFPE Tissue Kit uses QIAamp® DNA Micro technology for purification of genomic and mitochondrial DNA.

In some instances, the disclosed methods may further comprise determining or acquiring a yield value for the nucleic acid extracted from the sample and comparing the determined value to a reference value. For example, if the determined or acquired value is less than the reference value, the nucleic acids may be amplified prior to proceeding with library construction. In some instances, the disclosed methods may further comprise determining or acquiring a value for the size (or average size) of nucleic acid fragments in the sample, and comparing the determined or acquired value to a reference value, e.g., a size (or average size) of at least 100, 200, 300, 400, 500, 600, 700, 800, 900, or 1000 base pairs (bps). In some instances, one or more parameters described herein may be adjusted or selected in response to this determination.

After isolation, the nucleic acids are typically dissolved in a slightly alkaline buffer, e.g., Tris-EDTA (TE) buffer, or in ultra-pure water. In some instances, the isolated nucleic acids (e.g., genomic DNA) may be fragmented or sheared by using any of a variety of techniques known to those of skill in the art. For example, genomic DNA can be fragmented by physical shearing methods, enzymatic cleavage methods, chemical cleavage methods, and other methods known to those of skill in the art. Methods for DNA shearing are described in Example 4 in International Patent Application Publication No. WO 2012/092426. In some instances, alternatives to DNA shearing methods can be used to avoid a ligation step during library preparation.

### Library Preparation

In some instances, the nucleic acids isolated from the sample may be used to construct a library (e.g., a nucleic acid library as described herein). In some instances, the nucleic acids are fragmented using any of the methods described above, optionally subjected to repair of chain end damage, and optionally ligated to synthetic adapters, primers, and/or barcodes (e.g., amplification primers, sequencing adapters, flow cell adapters, substrate adapters, sample barcodes or indexes, and/or unique molecular identifier sequences), size-selected (e.g., by preparative gel electrophoresis), and/or amplified (e.g., using PCR, a non-PCR amplification technique, or an isothermal amplification technique). In some instances, the fragmented and adapter-ligated group of nucleic acids is used without explicit size selection or amplification prior to hybridization-based selection of target sequences. In some instances, the nucleic acid is amplified by any of a variety of specific or non-specific nucleic acid amplification methods known to those of skill in the art. In some instances, the nucleic acids are amplified, e.g., by a whole-genome amplification method such as random-primed strand-displacement amplification. Examples of nucleic acid library preparation techniques for next-generation sequencing are described in, e.g., van Dijk, et al. (2014), Exp. Cell Research 322:12-20, and Illumina's genomic DNA sample preparation kit.

In some instances, the resulting nucleic acid library may contain all or substantially all of the complexity of the genome. The term “substantially all” in this context refers to the possibility that there can in practice be some unwanted loss of genome complexity during the initial steps of the procedure. The methods described herein also are useful in cases where the nucleic acid library comprises a portion of the genome, e.g., where the complexity of the genome is reduced by design. In some instances, any selected portion of the genome can be used with a method described herein. For example, in certain embodiments, the entire exome or a subset thereof is isolated. In some instances, the library may include at least 95%, 90%, 80%, 70%, 60%, 50%, 40%, 30%, 20%, 10%, or 5% of the genomic DNA. In some instances, the library may consist of cDNA copies of genomic DNA that includes copies of at least 95%, 90%, 80%, 70%, 60%, 50%, 40%, 30%, 20%, 10%, or 5% of the genomic DNA. In certain instances, the amount of nucleic acid used to generate the nucleic acid library may be less than 5 micrograms, less than 1 microgram, less than 500 ng, less than 200 ng, less than 100 ng, less than 50 ng, less than 10 ng, less than 5 ng, or less than 1 ng.

In some instances, a library (e.g., a nucleic acid library) includes a collection of nucleic acid molecules. As described herein, the nucleic acid molecules of the library can include a target nucleic acid molecule (e.g., a tumor nucleic acid molecule, a reference nucleic acid molecule and/or a control nucleic acid molecule; also referred to herein as a first, second and/or third nucleic acid molecule, respectively). The nucleic acid molecules of the library can be from a single subject or individual. In some instances, a library can comprise nucleic acid molecules derived from more than one subject (e.g., 2, 3, 4, 5, 6, 7, 8, 9, 10, 20, 30 or more subjects). For example, two or more libraries from different subjects can be combined to form a library having nucleic acid molecules from more than one subject (where the nucleic acid molecules derived from each subject are optionally ligated to a unique sample barcode corresponding to a specific subject). In some instances, the subject is a human having, or at risk of having, a cancer or tumor.

In some instances, the library (or a portion thereof) may comprise one or more subgenomic intervals. In some instances, a subgenomic interval can be a single nucleotide position, e.g., a nucleotide position for which a variant at the position is associated (positively or negatively) with a tumor phenotype. In some instances, a subgenomic interval comprises more than one nucleotide position. Such instances include sequences of at least 2, 5, 10, 50, 100, 150, 250, or more than 250 nucleotide positions in length. Subgenomic intervals can comprise, e.g., one or more entire genes (or portions thereof), one or more exons or coding sequences (or portions thereof), one or more introns (or portion thereof), one or more microsatellite region (or portions thereof), or any combination thereof. A subgenomic interval can comprise all or a part of a fragment of a naturally occurring nucleic acid molecule, e.g., a genomic DNA molecule. For example, a subgenomic interval can correspond to a fragment of genomic DNA which is subjected to a sequencing reaction. In some instances, a subgenomic interval is a continuous sequence from a genomic source. In some instances, a subgenomic interval includes sequences that are not contiguous in the genome, e.g., subgenomic intervals in cDNA can include exon-exon junctions formed as a result of splicing. In some instances, the subgenomic interval comprises a tumor nucleic acid molecule. In some instances, the subgenomic interval comprises a non-tumor nucleic acid molecule.

### Targeting Gene Loci for Analysis

The methods described herein can be used in combination with, or as part of, a method for evaluating a plurality or set of subject intervals (e.g., target sequences), e.g., from a set of genomic loci (e.g., gene loci or fragments thereof), as described herein.

In some instances, the set of genomic loci evaluated by the disclosed methods comprises a plurality of, e.g., genes, which in mutant form, are associated with an effect on cell division, growth or survival, or are associated with a cancer, e.g., a cancer described herein.

In some instances, the set of gene loci evaluated by the disclosed methods comprises at least 1, at least 2, at least 3, at least 4, at least 5, at least 6, at least 7, at least 8, at least 9, at least 10, at least 20, at least 30, at least 40, at least 50, at least 60, at least 70, at least 80, at least 90, at least 100, or more than 100 gene loci.

In some instances, the selected gene loci (also referred to herein as target gene loci or target sequences), or fragments thereof, may include subject intervals comprising non-coding sequences, coding sequences, intragenic regions, or intergenic regions of the subject genome. For example, the subject intervals can include a non-coding sequence or fragment thereof (e.g., a promoter sequence, enhancer sequence, 5′ untranslated region (5′ UTR), 3′ untranslated region (3′ UTR), or a fragment thereof), a coding sequence of fragment thereof, an exon sequence or fragment thereof, an intron sequence or a fragment thereof.

### Target Capture Reagents

The methods described herein may comprise contacting a nucleic acid library with a plurality of target capture reagents in order to select and capture a plurality of specific target sequences (e.g., gene sequences or fragments thereof) for analysis. In some instances, a target capture reagent (i.e., a molecule which can bind to and thereby allow capture of a target molecule) is used to select the subject intervals to be analyzed. For example, a target capture reagent can be a bait molecule, e.g., a nucleic acid molecule (e.g., a DNA molecule or RNA molecule) which can hybridize to (i.e., is complementary to) a target molecule, and thereby allows capture of the target nucleic acid. In some instances, the target capture reagent, e.g., a bait molecule (or bait sequence), is a capture oligonucleotide (or capture probe). In some instances, the target nucleic acid is a genomic DNA molecule, an RNA molecule, a cDNA molecule derived from an RNA molecule, a microsatellite DNA sequence, and the like. In some instances, the target capture reagent is suitable for solution-phase hybridization to the target. In some instances, the target capture reagent is suitable for solid-phase hybridization to the target. In some instances, the target capture reagent is suitable for both solution-phase and solid-phase hybridization to the target. The design and construction of target capture reagents is described in more detail in, e.g., International Patent Application Publication No. WO 2020/236941, the entire content of which is incorporated herein by reference.

The methods described herein provide for optimized sequencing of a large number of genomic loci (e.g., genes or gene products (e.g., mRNA), microsatellite loci, etc.) from samples (e.g., cancerous tissue specimens, liquid biopsy samples, and the like) from one or more subjects by the appropriate selection of target capture reagents to select the target nucleic acid molecules to be sequenced. In some instances, a target capture reagent may hybridize to a specific target locus, e.g., a specific target gene locus or fragment thereof. In some instances, a target capture reagent may hybridize to a specific group of target loci, e.g., a specific group of gene loci or fragments thereof. In some instances, a plurality of target capture reagents comprising a mix of target-specific and/or group-specific target capture reagents may be used.

In some instances, the number of target capture reagents (e.g., bait molecules) in the plurality of target capture reagents (e.g., a bait set) contacted with a nucleic acid library to capture a plurality of target sequences for nucleic acid sequencing is greater than 10, greater than 50, greater than 100, greater than 200, greater than 300, greater than 400, greater than 500, greater than 600, greater than 700, greater than 800, greater than 900, greater than 1,000, greater than 1,250, greater than 1,500, greater than 1,750, greater than 2,000, greater than 3,000, greater than 4,000, greater than 5,000, greater than 10,000, greater than 25,000, or greater than 50,000.

In some instances, the overall length of the target capture reagent sequence can be between about 70 nucleotides and 1000 nucleotides. In one instance, the target capture reagent length is between about 100 and 300 nucleotides, 110 and 200 nucleotides, or 120 and 170 nucleotides, in length. In addition to those mentioned above, intermediate oligonucleotide lengths of about 70, 80, 90, 100, 110, 120, 130, 140, 150, 160, 170, 180, 190, 200, 210, 220, 230, 240, 250, 300, 400, 500, 600, 700, 800, and 900 nucleotides in length can be used in the methods described herein. In some embodiments, oligonucleotides of about 70, 80, 90, 100, 110, 120, 130, 140, 150, 160, 170, 180, 190, 200, 210, 220, or 230 bases can be used.

In some instances, each target capture reagent sequence can include: (i) a target-specific capture sequence (e.g., a gene locus or microsatellite locus-specific complementary sequence), (ii) an adapter, primer, barcode, and/or unique molecular identifier sequence, and (iii) universal tails on one or both ends. As used herein, the term “target capture reagent” can refer to the target-specific target capture sequence or to the entire target capture reagent oligonucleotide including the target-specific target capture sequence.

In some instances, the target-specific capture sequences in the target capture reagents are between about 40 nucleotides and 1000 nucleotides in length. In some instances, the target-specific capture sequence is between about 70 nucleotides and 300 nucleotides in length. In some instances, the target-specific sequence is between about 100 nucleotides and 200 nucleotides in length. In yet other instances, the target-specific sequence is between about 120 nucleotides and 170 nucleotides in length, typically 120 nucleotides in length. Intermediate lengths in addition to those mentioned above also can be used in the methods described herein, such as target-specific sequences of about 40, 50, 60, 70, 80, 90, 100, 110, 120, 130, 140, 150, 160, 170, 180, 190, 200, 210, 220, 230, 240, 250, 300, 400, 500, 600, 700, 800, and 900 nucleotides in length, as well as target-specific sequences of lengths between the above-mentioned lengths.

In some instances, the target capture reagent may be designed to select a subject interval containing one or more rearrangements, e.g., an intron containing a genomic rearrangement. In such instances, the target capture reagent is designed such that repetitive sequences are masked to increase the selection efficiency. In those instances where the rearrangement has a known juncture sequence, complementary target capture reagents can be designed to recognize the juncture sequence to increase the selection efficiency.

In some instances, the disclosed methods may comprise the use of target capture reagents designed to capture two or more different target categories, each category having a different target capture reagent design strategy. In some instances, the hybridization-based capture methods and target capture reagent compositions disclosed herein may provide for the capture and homogeneous coverage of a set of target sequences, while minimizing coverage of genomic sequences outside of the targeted set of sequences. In some instances, the target sequences may include the entire exome of genomic DNA or a selected subset thereof. In some instances, the target sequences may include, e.g., a large chromosomal region (e.g., a whole chromosome arm). The methods and compositions disclosed herein provide different target capture reagents for achieving different sequencing depths and patterns of coverage for complex sets of target nucleic acid sequences.

Typically, DNA molecules are used as target capture reagent sequences, although RNA molecules can also be used. In some instances, a DNA molecule target capture reagent can be single stranded DNA (ssDNA) or double-stranded DNA (dsDNA). In some instances, an RNA-DNA duplex is more stable than a DNA-DNA duplex and therefore provides for potentially better capture of nucleic acids.

In some instances, the disclosed methods comprise providing a selected set of nucleic acid molecules (e.g., a library catch) captured from one or more nucleic acid libraries. For example, the method may comprise: providing one or a plurality of nucleic acid libraries, each comprising a plurality of nucleic acid molecules (e.g., a plurality of target nucleic acid molecules and/or reference nucleic acid molecules) extracted from one or more samples from one or more subjects; contacting the one or a plurality of libraries (e.g., in a solution-based hybridization reaction) with one, two, three, four, five, or more than five pluralities of target capture reagents (e.g., oligonucleotide target capture reagents) to form a hybridization mixture comprising a plurality of target capture reagent/nucleic acid molecule hybrids; separating the plurality of target capture reagent/nucleic acid molecule hybrids from said hybridization mixture, e.g., by contacting said hybridization mixture with a binding entity that allows for separation of said plurality of target capture reagent/nucleic acid molecule hybrids from the hybridization mixture, thereby providing a library catch (e.g., a selected or enriched subgroup of nucleic acid molecules from the one or a plurality of libraries).

In some instances, the disclosed methods may further comprise amplifying the library catch (e.g., by performing PCR). In other instances, the library catch is not amplified.

In some instances, the target capture reagents can be part of a kit which can optionally comprise instructions, standards, buffers or enzymes or other reagents.

### Hybridization Conditions

As noted above, the methods disclosed herein may include the step of contacting the library (e.g., the nucleic acid library) with a plurality of target capture reagents to provide a selected library target nucleic acid sequences (i.e., the library catch). The contacting step can be effected in, e.g., solution-based hybridization. In some instances, the method includes repeating the hybridization step for one or more additional rounds of solution-based hybridization. In some instances, the method further includes subjecting the library catch to one or more additional rounds of solution-based hybridization with the same or a different collection of target capture reagents.

In some instances, the contacting step is effected using a solid support, e.g., an array. Suitable solid supports for hybridization are described in, e.g., Albert, T. J. et al. (2007) Nat. Methods 4(11):903-5; Hodges, E. et al. (2007) Nat. Genet. 39(12):1522-7; and Okou, D. T. et al. (2007) Nat. Methods 4(11):907-9, the contents of which are incorporated herein by reference in their entireties.

Hybridization methods that can be adapted for use in the methods herein are described in the art, e.g., as described in International Patent Application Publication No. WO 2012/092426. Methods for hybridizing target capture reagents to a plurality of target nucleic acids are described in more detail in, e.g., International Patent Application Publication No. WO 2020/236941, the entire content of which is incorporated herein by reference.

### Sequencing Methods

The methods and systems disclosed herein can be used in combination with, or as part of, a method or system for sequencing nucleic acids (e.g., a next-generation sequencing system) to generate a plurality of sequence reads that overlap one or more gene loci within a subgenomic interval in the sample and thereby determine, e.g., gene allele sequences at a plurality of gene loci. “Next-generation sequencing” (or “NGS”) as used herein may also be referred to as “massively parallel sequencing” (or “MPS”), and refers to any sequencing method that determines the nucleotide sequence of either individual nucleic acid molecules (e.g., as in single molecule sequencing) or clonally expanded proxies for individual nucleic acid molecules in a high throughput fashion (e.g., wherein greater than 10³, 10⁴, 10⁵ or more than 10⁵ molecules are sequenced simultaneously).

Next-generation sequencing methods are known in the art, and are described in, e.g., Metzker, M. (2010) Nature Biotechnology Reviews 11:31-46, which is incorporated herein by reference. Other examples of sequencing methods suitable for use when implementing the methods and systems disclosed herein are described in, e.g., International Patent Application Publication No. WO 2012/092426. In some instances, the sequencing may comprise, for example, whole genome sequencing (WGS), whole exome sequencing, targeted sequencing, or direct sequencing. In some instances, sequencing may be performed using, e.g., Sanger sequencing. In some instances, the sequencing may comprise a paired-end sequencing technique that allows both ends of a fragment to be sequenced and generates high-quality, alignable sequence data for detection of, e.g., genomic rearrangements, repetitive sequence elements, gene fusions, and novel transcripts.

The disclosed methods and systems may be implemented using sequencing platforms such as the Roche/454 Genome Sequencer (GS) FLX System, Illumina/Solexa Genome Analyzer (GA), Illumina's HiSeq® 2500, HiSeq® 3000, HiSeq® 4000 and NovaSeq® 6000 Sequencing Systems, Life/APG's Support Oligonucleotide Ligation Detection (SOLID) system, Polonator's G.007 system, Helicos BioSciences' HeliScope Gene Sequencing system, or Pacific Biosciences' PacBio® RS platform. In some instances, sequencing may comprise Illumina MiSeq™ sequencing. In some instances, sequencing may comprise Illumina HiSeq® sequencing. In some instances, sequencing may comprise Illumina NovaSeq® sequencing. Optimized methods for sequencing a large number of target genomic loci in nucleic acids extracted from a sample are described in more detail in, e.g., International Patent Application Publication No. WO 2020/236941, the entire content of which is incorporated herein by reference.

In certain instances, the disclosed methods comprise one or more of the steps of: (a) acquiring a library comprising a plurality of normal and/or tumor nucleic acid molecules from a sample; (b) simultaneously or sequentially contacting the library with one, two, three, four, five, or more than five pluralities of target capture reagents under conditions that allow hybridization of the target capture reagents to the target nucleic acid molecules, thereby providing a selected set of captured normal and/or tumor nucleic acid molecules (i.e., a library catch); (c) separating the selected subset of the nucleic acid molecules (e.g., the library catch) from the hybridization mixture, e.g., by contacting the hybridization mixture with a binding entity that allows for separation of the target capture reagent/nucleic acid molecule hybrids from the hybridization mixture, (d) sequencing the library catch to acquiring a plurality of reads (e.g., sequence reads) that overlap one or more subject intervals (e.g., one or more target sequences) from said library catch that may comprise a mutation (or alteration), e.g., a variant sequence comprising a somatic mutation or germline mutation; (e) aligning said sequence reads using an alignment method as described elsewhere herein; and/or (f) assigning a nucleotide value for a nucleotide position in the subject interval (e.g., calling a mutation using, e.g., a Bayesian method or other method described herein) from one or more sequence reads of the plurality.

In some instances, acquiring sequence reads for one or more subject intervals may comprise sequencing at least 1, at least 5, at least 10, at least 20, at least 30, at least 40, at least 50, at least 100, at least 150, at least 200, at least 250, at least 300, at least 350, at least 400, at least 450, at least 500, at least 550, at least 600, at least 650, at least 700, at least 750, at least 800, at least 850, at least 900, at least 950, at least 1,000, at least 1,250, at least 1,500, at least 1,750, at least 2,000, at least 2,250, at least 2,500, at least 2,750, at least 3,000, at least 3,500, at least 4,000, at least 4,500, or at least 5,000 loci, e.g., genomic loci, gene loci, microsatellite loci, etc. In some instances, acquiring a sequence read for one or more subject intervals may comprise sequencing a subject interval for any number of loci within the range described in this paragraph, e.g., for at least 2,850 gene loci.

In some instances, acquiring a sequence read for one or more subject intervals comprises sequencing a subject interval with a sequencing method that provides a sequence read length (or average sequence read length) of at least 20 bases, at least 30 bases, at least 40 bases, at least 50 bases, at least 60 bases, at least 70 bases, at least 80 bases, at least 90 bases, at least 100 bases, at least 120 bases, at least 140 bases, at least 160 bases, at least 180 bases, at least 200 bases, at least 220 bases, at least 240 bases, at least 260 bases, at least 280 bases, at least 300 bases, at least 320 bases, at least 340 bases, at least 360 bases, at least 380 bases, or at least 400 bases. In some instances, acquiring a sequence read for the one or more subject intervals may comprise sequencing a subject interval with a sequencing method that provides a sequence read length (or average sequence read length) of any number of bases within the range described in this paragraph, e.g., a sequence read length (or average sequence read length) of 56 bases.

In some instances, acquiring a sequence read for one or more subject intervals may comprise sequencing with at least 100× or more coverage (or depth) on average. In some instances, acquiring a sequence read for one or more subject intervals may comprise sequencing with at least 100×, at least 150×, at least 200×, at least 250×, at least 500×, at least 750×, at least 1,000×, at least 1,500×, at least 2,000×, at least 2,500×, at least 3,000×, at least 3,500×, at least 4,000×, at least 4,500×, at least 5,000×, at least 5,500×, or at least 6,000× or more coverage (or depth) on average. In some instances, acquiring a sequence read for one or more subject intervals may comprise sequencing with an average coverage (or depth) having any value within the range of values described in this paragraph, e.g., at least 160×.

In some instances, acquiring a read for the one or more subject intervals comprises sequencing with an average sequencing depth having any value ranging from at least 100× to at least 6,000× for greater than about 90%, 92%, 94%, 95%, 96%, 97%, 98%, or 99% of the gene loci sequenced. For example, in some instances acquiring a read for the subject interval comprises sequencing with an average sequencing depth of at least 125× for at least 99% of the gene loci sequenced. As another example, in some instances acquiring a read for the subject interval comprises sequencing with an average sequencing depth of at least 4,100× for at least 95% of the gene loci sequenced.

In some instances, the relative abundance of a nucleic acid species in the library can be estimated by counting the relative number of occurrences of their cognate sequences (e.g., the number of sequence reads for a given cognate sequence) in the data generated by the sequencing experiment.

In some instances, the disclosed methods and systems provide nucleotide sequences for a set of subject intervals (e.g., gene loci), as described herein. In certain instances, the sequences are provided without using a method that includes a matched normal control (e.g., a wild-type control) and/or a matched tumor control (e.g., primary versus metastatic).

In some instances, the level of sequencing depth as used herein (e.g., an X-fold level of sequencing depth) refers to the number of reads (e.g., unique reads) obtained after detection and removal of duplicate reads (e.g., PCR duplicate reads). In other instances, duplicate reads are evaluated, e.g., to support detection of copy number alteration (CNAs).

### Alignment

Alignment is the process of matching a read with a location, e.g., a genomic location or locus. In some instances, NGS reads may be aligned to a known reference sequence (e.g., a wild-type sequence). In some instances, NGS reads may be assembled de novo. Methods of sequence alignment for NGS reads are described in, e.g., Trapnell, C. and Salzberg, S. L. Nature Biotech., 2009, 27:455-457. Examples of de novo sequence assemblies are described in, e.g., Warren R., et al., Bioinformatics, 2007, 23:500-501; Butler, J. et al., Genome Res., 2008, 18:810-820; and Zerbino, D. R. and Birney, E., Genome Res., 2008, 18:821-829. Optimization of sequence alignment is described in the art, e.g., as set out in International Patent Application Publication No. WO 2012/092426. Additional description of sequence alignment methods is provided in, e.g., International Patent Application Publication No. WO 2020/236941, the entire content of which is incorporated herein by reference.

Misalignment (e.g., the placement of base-pairs from a short read at incorrect locations in the genome), e.g., misalignment of reads due to sequence context (e.g., the presence of repetitive sequence) around an actual cancer mutation can lead to reduction in sensitivity of mutation detection, can lead to a reduction in sensitivity of mutation detection, as reads for the alternate allele may be shifted off the histogram peak of alternate allele reads. Other examples of sequence context that may cause misalignment include short-tandem repeats, interspersed repeats, low complexity regions, insertions-deletions (indels), and paralogs. If the problematic sequence context occurs where no actual mutation is present, misalignment may introduce artifactual reads of “mutated” alleles by placing reads of actual reference genome base sequences at the wrong location. Because mutation-calling algorithms for multigene analysis should be sensitive to even low-abundance mutations, sequence misalignments may increase false positive discovery rates and/or reduce specificity.

In some instances, the methods and systems disclosed herein may integrate the use of multiple, individually-tuned, alignment methods or algorithms to optimize base-calling performance in sequencing methods, particularly in methods that rely on massively parallel sequencing (MPS) of a large number of diverse genetic events at a large number of diverse genomic loci. In some instances, the disclosed methods and systems may comprise the use of one or more global alignment algorithms. In some instances, the disclosed methods and systems may comprise the use of one or more local alignment algorithms. Examples of alignment algorithms that may be used include, but are not limited to, the Burrows-Wheeler Alignment (BWA) software bundle (see, e.g., Li, et al. (2009), “Fast and Accurate Short Read Alignment with Burrows-Wheeler Transform”, Bioinformatics 25:1754-60; Li, et al. (2010), Fast and Accurate Long-Read Alignment with Burrows-Wheeler Transform”, Bioinformatics epub. PMID: 20080505), the Smith-Waterman algorithm (see, e.g., Smith, et al. (1981), “Identification of Common Molecular Subsequences”, J. Molecular Biology 147(1):195-197), the Striped Smith-Waterman algorithm (see, e.g., Farrar (2007), “Striped Smith-Waterman Speeds Database Searches Six Times Over Other SIMD Implementations”, Bioinformatics 23(2):156-161), the Needleman-Wunsch algorithm (Needleman, et al. (1970) “A General Method Applicable to the Search for Similarities in the Amino Acid Sequence of Two Proteins”, J. Molecular Biology 48(3):443-53), or any combination thereof.

In some instances, the methods and systems disclosed herein may also comprise the use of a sequence assembly algorithm, e.g., the Arachne sequence assembly algorithm (see, e.g., Batzoglou, et al. (2002), “ARACHNE: A Whole-Genome Shotgun Assembler”, Genome Res. 12:177-189).

In some instances, the alignment method used to analyze sequence reads is not individually customized or tuned for detection of different variants (e.g., point mutations, insertions, deletions, and the like) at different genomic loci. In some instances, different alignment methods are used to analyze reads that are individually customized or tuned for detection of at least a subset of the different variants detected at different genomic loci. In some instances, different alignment methods are used to analyze reads that are individually customized or tuned to detect each different variant at different genomic loci. In some instances, tuning can be a function of one or more of: (i) the genetic locus (e.g., gene loci, microsatellite locus, or other subject interval) being sequenced, (ii) the tumor type associated with the sample, (iii) the variant being sequenced, or (iv) a characteristic of the sample or the subject. The selection or use of alignment conditions that are individually tuned to a number of specific subject intervals to be sequenced allows optimization of speed, sensitivity, and specificity. The method is particularly effective when the alignment of reads for a relatively large number of diverse subject intervals are optimized.

In some instances, the method includes the use of an alignment method optimized for rearrangements in combination with other alignment methods optimized for subject intervals not associated with rearrangements.

In some instances, the methods disclosed herein further comprise selecting or using an alignment method for analyzing, e.g., aligning, a sequence read, wherein said alignment method is a function of, is selected responsive to, or is optimized for, one or more of: (i) tumor type, e.g., the tumor type in the sample; (ii) the location (e.g., a gene locus) of the subject interval being sequenced; (iii) the type of variant (e.g., a point mutation, insertion, deletion, substitution, copy number variation (CNV), rearrangement, or fusion) in the subject interval being sequenced; (iv) the site (e.g., nucleotide position) being analyzed; (v) the type of sample (e.g., a sample described herein); and/or (vi) adjacent sequence(s) in or near the subject interval being evaluated (e.g., according to the expected propensity thereof for misalignment of the subject interval due to, e.g., the presence of repeated sequences in or near the subject interval).

In some instances, the methods disclosed herein allow for the rapid and efficient alignment of troublesome reads, e.g., a read having a rearrangement. Thus, in some instances where a read for a subject interval comprises a nucleotide position with a rearrangement, e.g., a translocation, the method can comprise using an alignment method that is appropriately tuned and that includes: (i) selecting a rearrangement reference sequence for alignment with a read, wherein said rearrangement reference sequence aligns with a rearrangement (in some instances, the reference sequence is not identical to the genomic rearrangement); and (ii) comparing, e.g., aligning, a read with said rearrangement reference sequence.

In some instances, alternative methods may be used to align troublesome reads. These methods are particularly effective when the alignment of reads for a relatively large number of diverse subject intervals is optimized. By way of example, a method of analyzing a sample can comprise: (i) performing a comparison (e.g., an alignment comparison) of a read using a first set of parameters (e.g., using a first mapping algorithm, or by comparison with a first reference sequence), and determining if said read meets a first alignment criterion (e.g., the read can be aligned with said first reference sequence, e.g., with less than a specific number of mismatches); (ii) if said read fails to meet the first alignment criterion, performing a second alignment comparison using a second set of parameters, (e.g., using a second mapping algorithm, or by comparison with a second reference sequence); and (iii) optionally, determining if said read meets said second criterion (e.g., the read can be aligned with said second reference sequence, e.g., with less than a specific number of mismatches), wherein said second set of parameters comprises use of, e.g., said second reference sequence, which, compared with said first set of parameters, is more likely to result in an alignment with a read for a variant (e.g., a rearrangement, insertion, deletion, or translocation).

In some instances, the alignment of sequence reads in the disclosed methods may be combined with a mutation calling method as described elsewhere herein. As discussed herein, reduced sensitivity for detecting actual mutations may be addressed by evaluating the quality of alignments (manually or in an automated fashion) around expected mutation sites in the genes or genomic loci (e.g., gene loci) being analyzed. In some instances, the sites to be evaluated can be obtained from databases of the human genome (e.g., the HG19 human reference genome) or cancer mutations (e.g., COSMIC). Regions that are identified as problematic can be remedied with the use of an algorithm selected to give better performance in the relevant sequence context, e.g., by alignment optimization (or re-alignment) using slower, but more accurate alignment algorithms such as Smith-Waterman alignment. In cases where general alignment algorithms cannot remedy the problem, customized alignment approaches may be created by, e.g., adjustment of maximum difference mismatch penalty parameters for genes with a high likelihood of containing substitutions; adjusting specific mismatch penalty parameters based on specific mutation types that are common in certain tumor types (e.g. C→T in melanoma); or adjusting specific mismatch penalty parameters based on specific mutation types that are common in certain sample types (e.g. substitutions that are common in FFPE).

Reduced specificity (increased false positive rate) in the evaluated subject intervals due to misalignment can be assessed by manual or automated examination of all mutation calls in the sequencing data. Those regions found to be prone to spurious mutation calls due to misalignment can be subjected to alignment remedies as discussed above. In cases where no algorithmic remedy is found possible, “mutations” from the problem regions can be classified or screened out from the panel of targeted loci.

### Mutation Calling

Base calling refers to the raw output of a sequencing device, e.g., the determined sequence of nucleotides in an oligonucleotide molecule. Mutation calling refers to the process of selecting a nucleotide value, e.g., A, G, T, or C, for a given nucleotide position being sequenced. Typically, the sequence reads (or base calling) for a position will provide more than one value, e.g., some reads will indicate a T and some will indicate a G. Mutation calling is the process of assigning a correct nucleotide value, e.g., one of those values, to the sequence. Although it is referred to as “mutation” calling, it can be applied to assign a nucleotide value to any nucleotide position, e.g., positions corresponding to mutant alleles, wild-type alleles, alleles that have not been characterized as either mutant or wild-type, or to positions not characterized by variability.

In some instances, the disclosed methods may comprise the use of customized or tuned mutation calling algorithms or parameters thereof to optimize performance when applied to sequencing data, particularly in methods that rely on massively parallel sequencing (MPS) of a large number of diverse genetic events at a large number of diverse genomic loci (e.g., gene loci, microsatellite regions, etc.) in samples, e.g., samples from a subject having cancer. Optimization of mutation calling is described in the art, e.g., as set out in International Patent Application Publication No. WO 2012/092426.

Methods for mutation calling can include one or more of the following: making independent calls based on the information at each position in the reference sequence (e.g., examining the sequence reads; examining the base calls and quality scores; calculating the probability of observed bases and quality scores given a potential genotype; and assigning genotypes (e.g., using Bayes' rule)); removing false positives (e.g., using depth thresholds to reject SNPs with read depth much lower or higher than expected; local realignment to remove false positives due to small indels); and performing linkage disequilibrium (LD)/imputation-based analysis to refine the calls.

Equations used to calculate the genotype likelihood associated with a specific genotype and position are described in, e.g., Li, H. and Durbin, R. Bioinformatics, 2010; 26(5): 589-95. The prior expectation for a particular mutation in a certain cancer type can be used when evaluating samples from that cancer type. Such likelihood can be derived from public databases of cancer mutations, e.g., Catalogue of Somatic Mutation in Cancer (COSMIC), HGMD (Human Gene Mutation Database), The SNP Consortium, Breast Cancer Mutation Data Base (BIC), and Breast Cancer Gene Database (BCGD).

Examples of LD/imputation based analysis are described in, e.g., Browning, B. L. and Yu, Z. Am. J. Hum. Genet. 2009, 85(6):847-61. Examples of low-coverage SNP calling methods are described in, e.g., Li, Y., et al., Annu. Rev. Genomics Hum. Genet. 2009, 10:387-406.

After alignment, detection of substitutions can be performed using a mutation calling method (e.g., a Bayesian mutation calling method) which is applied to each base in each of the subject intervals, e.g., exons of a gene or other locus to be evaluated, where presence of alternate alleles is observed. This method will compare the probability of observing the read data in the presence of a mutation with the probability of observing the read data in the presence of base-calling error alone. Mutations can be called if this comparison is sufficiently strongly supportive of the presence of a mutation.

An advantage of a Bayesian mutation detection approach is that the comparison of the probability of the presence of a mutation with the probability of base-calling error alone can be weighted by a prior expectation of the presence of a mutation at the site. If some reads of an alternate allele are observed at a frequently mutated site for the given cancer type, then presence of a mutation may be confidently called even if the amount of evidence of mutation does not meet the usual thresholds. This flexibility can then be used to increase detection sensitivity for even rarer mutations/lower purity samples, or to make the test more robust to decreases in read coverage. The likelihood of a random base-pair in the genome being mutated in cancer is ~1e-6. The likelihood of specific mutations occurring at many sites in, for example, a typical multigenic cancer genome panel can be orders of magnitude higher. These likelihoods can be derived from public databases of cancer mutations (e.g., COSMIC).

Indel calling is a process of finding bases in the sequencing data that differ from the reference sequence by insertion or deletion, typically including an associated confidence score or statistical evidence metric. Methods of indel calling can include the steps of identifying candidate indels, calculating genotype likelihood through local re-alignment, and performing LD-based genotype inference and calling. Typically, a Bayesian approach is used to obtain potential indel candidates, and then these candidates are tested together with the reference sequence in a Bayesian framework.

Algorithms to generate candidate indels are described in, e.g., McKenna, A., et al., Genome Res. 2010; 20(9):1297-303; Ye, K., et al., Bioinformatics, 2009; 25(21):2865-71; Lunter, G., and Goodson, M., Genome Res. 2011; 21(6):936-9; and Li, H., et al. (2009), Bioinformatics 25(16):2078-9.

Methods for generating indel calls and individual-level genotype likelihoods include, e.g., the Dindel algorithm (Albers, C. A., et al., Genome Res. 2011; 21(6):961-73). For example, the Bayesian EM algorithm can be used to analyze the reads, make initial indel calls, and generate genotype likelihoods for each candidate indel, followed by imputation of genotypes using, e.g., QCALL (Le S. Q. and Durbin R. Genome Res. 2011; 21(6):952-60). Parameters, such as prior expectations of observing the indel can be adjusted (e.g., increased or decreased), based on the size or location of the indels.

Methods have been developed that address limited deviations from allele frequencies of 50% or 100% for the analysis of cancer DNA. (see, e.g., SNVMix—Bioinformatics. 2010 Mar. 15; 26(6): 730-736.) Methods disclosed herein, however, allow consideration of the possibility of the presence of a mutant allele at frequencies (or allele fractions) ranging from 1% to 100% (i.e., allele fractions ranging from 0.01 to 1.0), and especially at levels lower than 50%. This approach is particularly important for the detection of mutations in, for example, low-purity FFPE samples of natural (multi-clonal) tumor DNA.

In some instances, the mutation calling method used to analyze sequence reads is not individually customized or fine-tuned for detection of different mutations at different genomic loci. In some instances, different mutation calling methods are used that are individually customized or fine-tuned for at least a subset of the different mutations detected at different genomic loci. In some instances, different mutation calling methods are used that are individually customized or fine-tuned for each different mutant detected at each different genomic loci. The customization or tuning can be based on one or more of the factors described herein, e.g., the type of cancer in a sample, the gene or locus in which the subject interval to be sequenced is located, or the variant to be sequenced. This selection or use of mutation calling methods individually customized or fine-tuned for a number of subject intervals to be sequenced allows for optimization of speed, sensitivity and specificity of mutation calling.

In some instances, a nucleotide value is assigned for a nucleotide position in each of X unique subject intervals using a unique mutation calling method, and X is at least 2, at least 3, at least 4, at least 5, at least 10, at least 15, at least 20, at least 30, at least 40, at least 50, at least 60, at least 70, at least 80, at least 90, at least 100, at least 200, at least 300, at least 400, at least 500, at least 1000, at least 1500, at least 2000, at least 2500, at least 3000, at least 3500, at least 4000, at least 4500, at least 5000, or greater. The calling methods can differ, and thereby be unique, e.g., by relying on different Bayesian prior values.

In some instances, assigning said nucleotide value is a function of a value which is or represents the prior (e.g., literature) expectation of observing a read showing a variant, e.g., a mutation, at said nucleotide position in a tumor of type.

In some instances, the method comprises assigning a nucleotide value (e.g., calling a mutation) for at least 10, 20, 40, 50, 60, 70, 80, 90, 100, 200, 300, 400, 500, 600, 700, 800, 900, or 1,000 nucleotide positions, wherein each assignment is a function of a unique value (as opposed to the value for the other assignments) which is or represents the prior (e.g., literature) expectation of observing a read showing a variant, e.g., a mutation, at said nucleotide position in a tumor of type.

In some instances, assigning said nucleotide value is a function of a set of values which represent the probabilities of observing a read showing said variant at said nucleotide position if the variant is present in the sample at a specified frequency (e.g., 1%, 5%, 10%, etc.) and/or if the variant is absent (e.g., observed in the reads due to base-calling error alone).

In some instances, the mutation calling methods described herein can include the following: (a) acquiring, for a nucleotide position in each of said X subject intervals: (i) a first value which is or represents the prior (e.g., literature) expectation of observing a read showing a variant, e.g., a mutation, at said nucleotide position in a tumor of type X; and (ii) a second set of values which represent the probabilities of observing a read showing said variant at said nucleotide position if the variant is present in the sample at a frequency (e.g., 1%, 5%, 10%, etc.) and/or if the variant is absent (e.g., observed in the reads due to base-calling error alone); and (b) responsive to said values, assigning a nucleotide value (e.g., calling a mutation) from said reads for each of said nucleotide positions by weighing, e.g., by a Bayesian method described herein, the comparison among the values in the second set using the first value (e.g., computing the posterior probability of the presence of a mutation), thereby analyzing said sample.

Additional description of exemplary nucleic acid sequencing methods, mutation calling methods, and methods for analysis of genetic variants is provided in, e.g., U.S. Pat. Nos. 9,340,830, 9,792,403, 11,136,619, 11,118,213, and International Patent Application Publication No. WO 2020/236941, the entire contents of each of which is incorporated herein by reference.

### Systems

Also disclosed herein are systems designed to implement any of the disclosed methods for detecting HJBs in a sample from a subject. The systems may comprise, e.g., one or more processors, and a memory unit communicatively coupled to the one or more processors and configured to store instructions that, when executed by the one or more processors, cause the system to: receive, using the one or more processors, genomic data from a sample from a subject; determine, using the one or more processors, a copy number at one or more genomic loci based on the genomic data; determine, using the one or more processors, a distance for each of the one or more genomic loci from a centromere of a chromosome; and determine a correlation, using the one or more processors, wherein the correlation comprises a change in the determined copy number as a function of the determined distances. In some aspects, the system comprises further instructions that, when executed by the one or more processors, cause the system to: detect a presence or an absence of a disease marker in the sample based on the correlation. In some aspects, the disease marker comprises a Howell-Jolly Body (HJB).

In some aspects, the system comprises further instructions that, when executed by the one or more processors, cause the system to: diagnose a disease in the subject based on the presence or the absence of the disease marker in the sample from the subject. In some aspects, the system comprises further instructions that, when executed by the one or more processors, cause the system to: treat the subject for the disease based on presence or the absence of the disease marker in the sample from the subject. In some aspects, the system comprises further instructions that, when executed by the one or more processors, cause the system to: perform quality control analysis on the received genomic data.

In some instances, the disclosed systems may further comprise a sequencer, e.g., a next generation sequencer (also referred to as a massively parallel sequencer). Examples of next generation (or massively parallel) sequencing platforms include, but are not limited to, Roche/454's Genome Sequencer (GS) FLX system, Illumina/Solexa's Genome Analyzer (GA), Illumina's HiSeq® 2500, HiSeq® 3000, HiSeq® 4000 and NovaSeq® 6000 sequencing systems, Life/APG's Support Oligonucleotide Ligation Detection (SOLID) system, Polonator's G.007 system, Helicos BioSciences' HeliScope Gene Sequencing system, ThermoFisher Scientific's Ion Torrent Genexus system, or Pacific Biosciences' PacBio® RS system.

In some instances, the disclosed systems may be used for detecting HJBs in any of a variety of samples as described herein (e.g., a tissue sample, biopsy sample, hematological sample, or liquid biopsy sample derived from the subject).

In some instances, the plurality of gene loci for which sequencing data is processed to detect HJBs may comprise at least 1, 2, 3, 4, 5, 6, 7, 8, 9, 10, 20, 30, 40, 50, 60, 70, 80, 0, 100, 200, 300, 400, 500, 600, 700, 800, 900, 1000, or more than 1000 gene loci (or any number of gene loci within the range of 1 to more than 1000 gene loci).

In some instance, the nucleic acid sequence data is acquired using a next generation sequencing technique (also referred to as a massively parallel sequencing technique) having a read-length of less than 400 bases, less than 300 bases, less than 200 bases, less than 150 bases, less than 100 bases, less than 90 bases, less than 80 bases, less than 70 bases, less than 60 bases, less than 50 bases, less than 40 bases, or less than 30 bases.

In some instances, the detection of HJBs is used to select, initiate, adjust, or terminate a treatment for cancer in the subject (e.g., a patient) from which the sample was derived, as described elsewhere herein.

In some instances, the disclosed systems may further comprise sample processing and library preparation workstations, microplate-handling robotics, fluid dispensing systems, temperature control modules, environmental control chambers, additional data storage modules, data communication modules (e.g., Bluetooth®, WiFi, intranet, or internet communication hardware and associated software), display modules, one or more local and/or cloud-based software packages (e.g., instrument/system control software packages, sequencing data analysis software packages), etc., or any combination thereof. In some instances, the systems may comprise, or be part of, a computer system or computer network as described elsewhere herein.

### Machine Learning

Any of a variety of machine learning approaches & algorithms (where a machine learning model, as referred to herein, comprises a trained machine learning algorithm) may be used in implementing the disclosed methods. For example, the machine learning model may comprise a supervised learning model (i.e., a model trained using labeled sets of training data), an unsupervised learning model (i.e., a model trained using unlabeled sets of training data), a semi-supervised learning model (i.e., a model trained using a combination of labeled and unlabeled training data), a self-supervised learning model, or any combination thereof. In some examples, the machine learning model can comprise a deep learning model (i.e., a model comprising many layers of coupled “nodes” that may be trained in a supervised, unsupervised, or semi-supervised manner).

In some instances, one or more machine learning models (e.g., 1, 2, 3, 4, 5, 6, 7, 8, 9, 10, or more than 10 machine learning models), or a combination thereof, may be utilized to implement the disclosed methods.

In some instances, the one or more machine learning models may comprise statistical methods for analyzing data. The machine learning models may be used for classification and/or regression of data. The machine learning models can include, for example, neural networks, support vector machines, decision trees, ensemble learning (e.g., bagging-based learning, such as random forest, and/or boosting-based learning), k-nearest neighbors algorithms, linear regression-based models, and/or logistic regression-based models. The machine learning models can comprise regularization, such as L1 regularization and/or L2 regularization. The machine learning models can include the use of dimensionality reduction techniques (e.g., principal component analysis, matrix factorization techniques, and/or autoencoders) and/or clustering techniques (e.g., hierarchical clustering, k-means clustering, distribution-based clustering, such as Gaussian mixture models, or density-based clustering, such as DBSCAN or OPTICS). The one or more machine learning models can comprise solving, e.g., optimizing, an objective function over multiple iterations based on a training data set. The iterative solving approach can be used even when the machine learning model comprises a model for which there exists a closed-form solution (e.g., linear regression).

In some instances, the machine learning models can comprise artificial neural networks (ANNs), e.g., deep learning models. For example, the one or more machine learning models/algorithms used for implementing the disclosed methods may include an ANN which can comprise any of a variety of computational motifs/architectures known to those of skill in the art, including, but not limited to, feedforward connections (e.g., skip connections), recurrent connections, fully connected layers, convolutional layers, and/or pooling functions (e.g., attention, including self-attention). The artificial neural networks can comprise differentiable non-linear functions trained by backpropagation.

Artificial neural networks, e.g., deep learning models, generally comprise an interconnected group of nodes organized into multiple layers of nodes. For example, the ANN architecture may comprise at least an input layer, one or more hidden layers (i.e., intermediate layers), and an output layer. The ANN or deep learning model may comprise any total number of layers (e.g., 3, 4, 5, 6, 7, 8, 9, 10, 15, 20, or more than 20 layers in total), and any number of hidden layers (e.g., 1, 2, 3, 4, 5, 6, 7, 8, 9, 10, 15, 20, or more than 20 hidden layers), where the hidden layers function as trainable feature extractors that allow mapping of a set of input data to a preferred output value or set of output values. Each layer of the neural network comprises a plurality of nodes (e.g., at least 10, 25, 50, 75 100, 200, 300, 400, 500, 600, 700, 800, 900, 1000, 2000, 3000, 4000, 5000, 6000, 7000, 8000, 9000, 10,000, or more than 10,000 nodes). A node receives input data (e.g., genomic feature data (such as variant sequence data, methylation status data, etc.), non-genomic feature data (e.g., digital pathology image feature data), or other types of input data (e.g., patient-specific clinical data)) that comes either directly from one or more input data nodes or from the output of one or more nodes in previous layers, and performs a specific operation, e.g., a summation operation. In some cases, a connection from an input to a node is associated with a weight (or weighting factor). In some cases, the node may, for example, sum up the products of all pairs of inputs, Xᵢ, and their associated weights, Wᵢ. In some cases, the weighted sum is offset with a bias, b. In some cases, the output of a node may be gated using a threshold or activation function, ƒ, where ƒ may be a linear or non-linear function. The activation function may be, for example, a rectified linear unit (ReLU) activation function or other function such as a saturating hyperbolic tangent, identity, binary step, logistic, arcTan, softsign, parameteric rectified linear unit, exponential linear unit, softPlus, bent identity, softExponential, Sinusoid, Sine, Gaussian, or sigmoid function, or any combination thereof.

The weighting factors, bias values, and threshold values, or other computational parameters of the neural network (or other machine learning architecture), can be “taught” or “learned” in a training phase using one or more sets of training data (e.g., 1, 2, 3, 4, 5, or more than 5 sets of training data) and a specified training approach configured to solve, e.g., minimize, a loss function. For example, the adjustable parameters for an ANN (e.g., deep learning model) may be determined based on input data from a training data set using an iterative solver (such as a gradient-based method, e.g., backpropagation), so that the output value(s) that the ANN computes (e.g., a classification of a sample or a prediction of a disease outcome) are consistent with the examples included in the training data set. The training of the model (i.e., determination of the adjustable parameters of the model using an iterative solver) may or may not be performed using the same hardware as that used for deployment of the trained model.

In some instances, the disclosed methods may comprise retraining any of the machine learning models (e.g., iteratively retraining a previously trained model using one or more training data sets that differ from those used to train the model initially). In some instances, retraining the machine learning model may comprise using a continuous, e.g., online, machine learning model, i.e., where the model is periodically or continuously updated or retrained based on new training data. The new training data may be provided by, e.g., a single deployed local operational system, a plurality of deployed local operational systems, or a plurality of deployed, geographically-distributed operational systems. In some instances, the disclosed methods may employ, for example, pre-trained ANNs, and the pre-trained ANNs can be fine-tuned according to an additional dataset that is inputted into the pre-trained ANN.

### Computer Systems and Networks

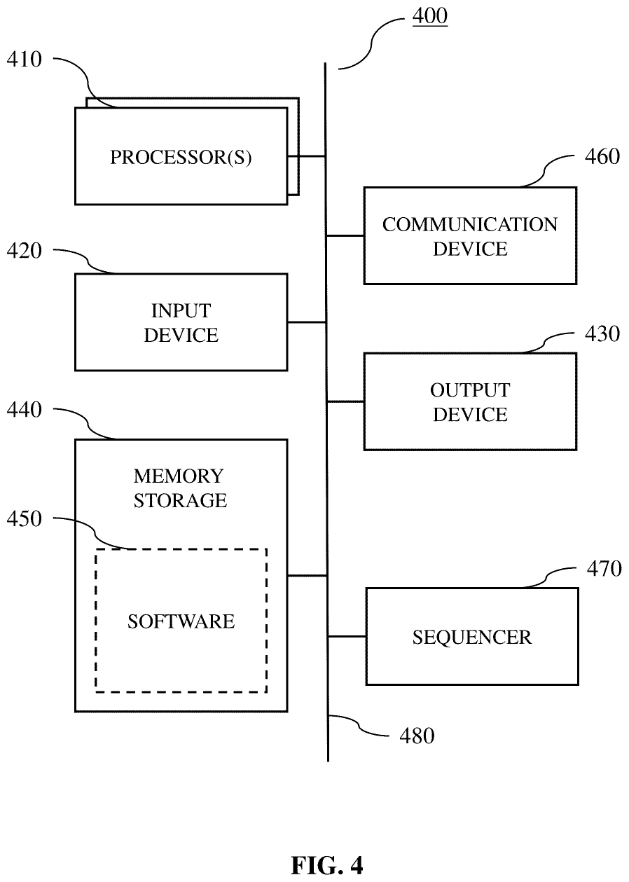

FIG. 4 illustrates an example of a computing device or system in accordance with one embodiment. Device 400 can be a host computer connected to a network. Device 400 can be a client computer or a server. As shown in FIG. 4, device 400 can be any suitable type of microprocessor-based device, such as a personal computer, workstation, server or handheld computing device (portable electronic device) such as a phone or tablet. The device can include, for example, one or more processor(s) 410, input devices 420, output devices 430, memory or storage devices 440, communication devices 460, and nucleic acid sequencers 470. Software 450 residing in memory or storage device 440 may comprise, e.g., an operating system as well as software for executing the methods described herein. Input device 420 and output device 430 can generally correspond to those described herein, and can either be connectable or integrated with the computer.

Input device 420 can be any suitable device that provides input, such as a touch screen, keyboard or keypad, mouse, or voice-recognition device. Output device 430 can be any suitable device that provides output, such as a touch screen, haptics device, or speaker.

Storage 440 can be any suitable device that provides storage (e.g., an electrical, magnetic or optical memory including a RAM (volatile and non-volatile), cache, hard drive, or removable storage disk). Communication device 460 can include any suitable device capable of transmitting and receiving signals over a network, such as a network interface chip or device. The components of the computer can be connected in any suitable manner, such as via a wired media (e.g., a physical system bus 480, Ethernet connection, or any other wire transfer technology) or wirelessly (e.g., Bluetooth®, Wi-Fi®, or any other wireless technology).

Software module 450, which can be stored as executable instructions in storage 440 and executed by processor(s) 410, can include, for example, an operating system and/or the processes that embody the functionality of the methods of the present disclosure (e.g., as embodied in the devices as described herein).

Software module 450 can also be stored and/or transported within any non-transitory computer-readable storage medium for use by or in connection with an instruction execution system, apparatus, or device, such as those described herein, that can fetch instructions associated with the software from the instruction execution system, apparatus, or device and execute the instructions. In the context of this disclosure, a computer-readable storage medium can be any medium, such as storage 440, that can contain or store processes for use by or in connection with an instruction execution system, apparatus, or device. Examples of computer-readable storage media may include memory units like hard drives, flash drives and distribute modules that operate as a single functional unit. Also, various processes described herein may be embodied as modules configured to operate in accordance with the embodiments and techniques described above. Further, while processes may be shown and/or described separately, those skilled in the art will appreciate that the above processes may be routines or modules within other processes.

Software module 450 can also be propagated within any transport medium for use by or in connection with an instruction execution system, apparatus, or device, such as those described above, that can fetch instructions associated with the software from the instruction execution system, apparatus, or device and execute the instructions. In the context of this disclosure, a transport medium can be any medium that can communicate, propagate or transport programming for use by or in connection with an instruction execution system, apparatus, or device. The transport readable medium can include, but is not limited to, an electronic, magnetic, optical, electromagnetic or infrared wired or wireless propagation medium.

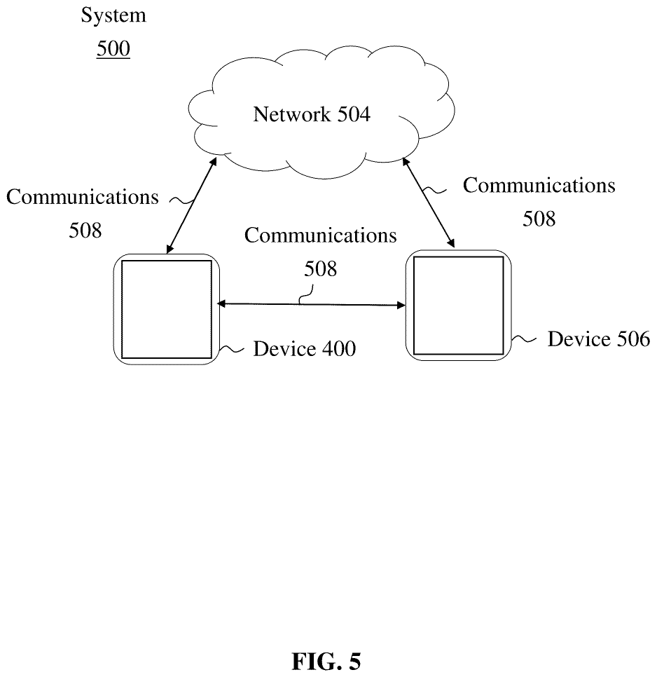

Device 400 may be connected to a network (e.g., network 504, as shown in FIG. 5 and/or described below), which can be any suitable type of interconnected communication system. The network can implement any suitable communications protocol and can be secured by any suitable security protocol. The network can comprise network links of any suitable arrangement that can implement the transmission and reception of network signals, such as wireless network connections, T1 or T3 lines, cable networks, DSL, or telephone lines.

Device 400 can be implemented using any operating system, e.g., an operating system suitable for operating on the network. Software module 450 can be written in any suitable programming language, such as C, C++, Java or Python. In various embodiments, application software embodying the functionality of the present disclosure can be deployed in different configurations, such as in a client/server arrangement or through a Web browser as a Web-based application or Web service, for example. In some embodiments, the operating system is executed by one or more processors, e.g., processor(s) 410.

Device 400 can further include a sequencer 470, which can be any suitable nucleic acid sequencing instrument.

FIG. 5 illustrates an example of a computing system in accordance with one embodiment. In system 500, device 400 (e.g., as described above and illustrated in FIG. 4) is connected to network 504, which is also connected to device 506. In some embodiments, device 506 is a sequencer. Exemplary sequencers can include, without limitation, Roche/454's Genome Sequencer (GS) FLX System, Illumina/Solexa's Genome Analyzer (GA), Illumina's HiSeq® 2500, HiSeq® 3000, HiSeq® 4000 and NovaSeq® 6000 Sequencing Systems, Life/APG's Support Oligonucleotide Ligation Detection (SOLID) system, Polonator's G.007 system, Helicos BioSciences' HeliScope Gene Sequencing system, Pacific Biosciences' PacBio® RS system, Ultima Genomics UG 50™ platform, or the Illumina NovaSeq X series platform.

Devices 400 and 506 may communicate, e.g., using suitable communication interfaces via network 504, such as a Local Area Network (LAN), Virtual Private Network (VPN), or the Internet. In some embodiments, network 504 can be, for example, the Internet, an intranet, a virtual private network, a cloud network, a wired network, or a wireless network. Devices 400 and 506 may communicate, in part or in whole, via wireless or hardwired communications, such as Ethernet, IEEE 802.11b wireless, or the like. Additionally, devices 400 and 506 may communicate, e.g., using suitable communication interfaces, via a second network, such as a mobile/cellular network. Communication between devices 400 and 506 may further include or communicate with various servers such as a mail server, mobile server, media server, telephone server, and the like. In some embodiments, Devices 400 and 506 can communicate directly (instead of, or in addition to, communicating via network 504), e.g., via wireless or hardwired communications, such as Ethernet, IEEE 802.11b wireless, or the like. In some embodiments, devices 400 and 506 communicate via communications 508, which can be a direct connection or can occur via a network (e.g., network 504).

One or all of devices 400 and 506 generally include logic (e.g., http web server logic) or are programmed to format data, accessed from local or remote databases or other sources of data and content, for providing and/or receiving information via network 504 according to various examples described herein.

### EXEMPLARY IMPLEMENTATIONS

Exemplary implementations of the methods and systems described herein include:
1. A method comprising:
   providing a plurality of nucleic acid molecules obtained from a sample from a subject;
   ligating one or more adapters onto one or more nucleic acid molecules from the plurality of nucleic acid molecules;
   amplifying the one or more ligated nucleic acid molecules from the plurality of nucleic acid molecules;
   capturing amplified nucleic acid molecules from the amplified nucleic acid molecules;
   sequencing, by a sequencer, the captured nucleic acid molecules to obtain a plurality of sequence reads that represent the captured nucleic acid molecules;
   receiving, using one or more processors, sequence read data for the plurality of sequence reads;
   determining, using the one or more processors, a copy number at one or more genomic loci based on the sequence read data;
   determining, using the one or more processors, a distance for each of the one or more genomic loci from a centromere of a chromosome; and
   determining a correlation, using the one or more processors, wherein the correlation comprises a change in the determined copy number as a function of the determined distances.
2. The method of clause 1, further comprising:
   detecting, using the one or more processors, a presence or an absence of a disease marker in the sample based on the correlation.
3. The method of clause 2, wherein the disease marker comprises a Howell-Jolly Body (HJB).
4. The method of clause 2 or clause 3, further comprising:
   diagnosing a disease in the subject based on the detecting of the presence or the absence of the disease marker in the sample from the subject.
5. The method of any of clauses 2-4, further comprising:
   treating the subject for the disease based on the detecting of the presence or the absence of the disease marker in the sample from the subject.
6. The method of clause 4 or clause 5, wherein the disease comprises asplenia, hyposplenia, or leukopenia.
7. The method of any of clauses 3-6, wherein the presence or the absence of HJBs informs a decision regarding treatment of the subject with an immunotherapy.
8. The method of clause 7, wherein the presence of HJBs informs a contraindication for an immunotherapy.
9. The method of any of clauses 1-8, wherein the correlation is a non-linear correlation.
10. The method of clause 9, wherein the non-linear correlation is an exponential correlation.
11. The method of any of clauses 1-10, wherein the correlation comprises an exponential increase in the copy number as a function of the distance for each of the one or more genomic loci from the centromere of the chromosome.
12. The method of any of clauses 1-8, wherein the correlation is a linear correlation.
13. The method of any of clauses 1-8 or 12, wherein the correlation comprises a linear increase in the copy number as a function of the distance for each of the one or more genomic loci from the centromere of the chromosome.
14. The method of any of clauses 1-13, wherein the subject is suspected of having or is determined to have cancer.
15. The method of clause 14, wherein the cancer is a B cell cancer (multiple myeloma), a melanoma, breast cancer, lung cancer, bronchus cancer, colorectal cancer, prostate cancer, pancreatic cancer, stomach cancer, ovarian cancer, urinary bladder cancer, brain cancer, central nervous system cancer, peripheral nervous system cancer, esophageal cancer, cervical cancer, uterine cancer, endometrial cancer, cancer of an oral cavity, cancer of a pharynx, liver cancer, kidney cancer, testicular cancer, biliary tract cancer, small bowel cancer, appendix cancer, salivary gland cancer, thyroid gland cancer, adrenal gland cancer, osteosarcoma, chondrosarcoma, a cancer of hematological tissue, an adenocarcinoma, an inflammatory myofibroblastic tumor, a gastrointestinal stromal tumor (GIST), colon cancer, multiple myeloma (MM), myelodysplastic syndrome (MDS), myeloproliferative disorder (MPD), acute lymphocytic leukemia (ALL), acute myelocytic leukemia (AML), chronic myelocytic leukemia (CML), chronic lymphocytic leukemia (CLL), polycythemia Vera, Hodgkin lymphoma, non-Hodgkin lymphoma (NHL), soft-tissue sarcoma, fibrosarcoma, myxosarcoma, liposarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, squamous cell carcinoma, basal cell carcinoma, adenocarcinoma, sweat gland carcinoma, sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, seminoma, embryonal carcinoma, Wilms' tumor, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, neuroblastoma, retinoblastoma, follicular lymphoma, diffuse large B-cell lymphoma, mantle cell lymphoma, hepatocellular carcinoma, thyroid cancer, gastric cancer, head and neck cancer, small cell cancer, essential thrombocythemia, agnogenic myeloid metaplasia, hypereosinophilic syndrome, systemic mastocytosis, familiar hypereosinophilia, chronic eosinophilic leukemia, neuroendocrine cancers, or a carcinoid tumor.
16. The method of clause 14, wherein the cancer comprises acute lymphoblastic leukemia (Philadelphia chromosome positive), acute lymphoblastic leukemia (precursor B-cell), acute myeloid leukemia (FLT3+), acute myeloid leukemia (with an IDH2 mutation), anaplastic large cell lymphoma, basal cell carcinoma, B-cell chronic lymphocytic leukemia, bladder cancer, breast cancer (HER2 overexpressed/amplified), breast cancer (HER2+), breast cancer (HR+, HER2−), cervical cancer, cholangiocarcinoma, chronic lymphocytic leukemia, chronic lymphocytic leukemia (with 17p deletion), chronic myelogenous leukemia, chronic myelogenous leukemia (Philadelphia chromosome positive), classical Hodgkin lymphoma, colorectal cancer, colorectal cancer (dMMR/MSI-H), colorectal cancer (KRAS wild type), cryopyrin-associated periodic syndrome, a cutaneous T-cell lymphoma, dermatofibrosarcoma protuberans, a diffuse large B-cell lymphoma, fallopian tube cancer, a follicular B-cell non-Hodgkin lymphoma, a follicular lymphoma, gastric cancer, gastric cancer (HER2+), gastroesophageal junction (GEJ) adenocarcinoma, a gastrointestinal stromal tumor, a gastrointestinal stromal tumor (KIT+), a giant cell tumor of the bone, a glioblastoma, granulomatosis with polyangiitis, a head and neck squamous cell carcinoma, a hepatocellular carcinoma, Hodgkin lymphoma, juvenile idiopathic arthritis, lupus erythematosus, a mantle cell lymphoma, medullary thyroid cancer, melanoma, a melanoma with a BRAF V600 mutation, a melanoma with a BRAF V600E or V600K mutation, Merkel cell carcinoma, multicentric Castleman's disease, multiple hematologic malignancies including Philadelphia chromosome-positive ALL and CML, multiple myeloma, myelofibrosis, a non-Hodgkin's lymphoma, a nonresectable subependymal giant cell astrocytoma associated with tuberous sclerosis, a non-small cell lung cancer, a non-small cell lung cancer (ALK+), a non-small cell lung cancer (PD-L1+), a non-small cell lung cancer (with ALK fusion or ROS1 gene alteration), a non-small cell lung cancer (with BRAF V600E mutation), a non-small cell lung cancer (with an EGFR exon 19 deletion or exon 21 substitution (L858R) mutations), a non-small cell lung cancer (with an EGFR T790M mutation), ovarian cancer, ovarian cancer (with a BRCA mutation), pancreatic cancer, a pancreatic, gastrointestinal, or lung origin neuroendocrine tumor, a pediatric neuroblastoma, a peripheral T-cell lymphoma, peritoneal cancer, prostate cancer, a renal cell carcinoma, rheumatoid arthritis, a small lymphocytic lymphoma, a soft tissue sarcoma, a solid tumor (MSI-H/dMMR), a squamous cell cancer of the head and neck, a squamous non-small cell lung cancer, thyroid cancer, a thyroid carcinoma, urothelial cancer, a urothelial carcinoma, or Waldenstrom's macroglobulinemia.
17. The method of clause 14, further comprising treating the subject with an anti-cancer therapy.
18. The method of clause 17, wherein the anti-cancer therapy comprises a targeted anti-cancer therapy.
19. The method of clause 18, wherein the targeted anti-cancer therapy comprises abemaciclib (Verzenio), abiraterone acetate (Zytiga), acalabrutinib (Calquence), ado-trastuzumab emtansine (Kadcyla), afatinib dimaleate (Gilotrif), alectinib (Alecensa), alemtuzumab (Campath), alitretinoin (Panretin), alpelisib (Piqray), amivantamab-vmjw (Rybrevant), anastrozole (Arimidex), apalutamide (Erleada), asciminib hydrochloride (Scemblix), atezolizumab (Tecentriq), avapritinib (Ayvakit), avelumab (Bavencio), axicabtagene ciloleucel (Yescarta), axitinib (Inlyta), belantamab mafodotin-blmf (Blenrep), belimumab (Benlysta), belinostat (Beleodaq), belzutifan (Welireg), bevacizumab (Avastin), bexarotene (Targretin), binimetinib (Mektovi), blinatumomab (Blincyto), bortezomib (Velcade), bosutinib (Bosulif), brentuximab vedotin (Adcetris), brexucabtagene autoleucel (Tecartus), brigatinib (Alunbrig), cabazitaxel (Jevtana), cabozantinib (Cabometyx), cabozantinib (Cabometyx, Cometriq), canakinumab (Ilaris), capmatinib hydrochloride (Tabrecta), carfilzomib (Kyprolis), cemiplimab-rwlc (Libtayo), ceritinib (LDK378/Zykadia), cetuximab (Erbitux), cobimetinib (Cotellic), crizotinib (Xalkori), dabrafenib (Tafinlar), dacomitinib (Vizimpro), daratumumab (Darzalex), daratumumab and hyaluronidase-fihj (Darzalex Faspro), darolutamide (Nubeqa), dasatinib (Sprycel), denileukin diftitox (Ontak), denosumab (Xgeva), dinutuximab (Unituxin), dostarlimab-gxly (Jemperli), durvalumab (Imfinzi), duvelisib (Copiktra), elotuzumab (Empliciti), enasidenib mesylate (Idhifa), encorafenib (Braftovi), enfortumab vedotin-ejfv (Padcev), entrectinib (Rozlytrek), enzalutamide (Xtandi), erdafitinib (Balversa), erlotinib (Tarceva), everolimus (Afinitor), exemestane (Aromasin), fam-trastuzumab deruxtecan-nxki (Enhertu), fedratinib hydrochloride (Inrebic), fulvestrant (Faslodex), gefitinib (Iressa), gemtuzumab ozogamicin (Mylotarg), gilteritinib (Xospata), glasdegib maleate (Daurismo), hyaluronidase-zzxf (Phesgo), ibrutinib (Imbruvica), ibritumomab tiuxetan (Zevalin), idecabtagene vicleucel (Abecma), idelalisib (Zydelig), imatinib mesylate (Gleevec), infigratinib phosphate (Truseltiq), inotuzumab ozogamicin (Besponsa), ipilimumab (Yervoy), isatuximab-irfc (Sarclisa), ivosidenib (Tibsovo), ixazomib citrate (Ninlaro), lanreotide acetate (Somatuline Depot), lapatinib (Tykerb), larotrectinib sulfate (Vitrakvi), lenvatinib mesylate (Lenvima), letrozole (Femara), lisocabtagene maraleucel (Breyanzi), loncastuximab tesirine-lpyl (Zynlonta), lorlatinib (Lorbrena), lutetium Lu 177-dotatate (Lutathera), margetuximab-cmkb (Margenza), midostaurin (Rydapt), mobocertinib succinate (Exkivity), mogamulizumab-kpkc (Poteligeo), moxetumomab pasudotox-tdfk (Lumoxiti), naxitamab-gqgk (Danyelza), necitumumab (Portrazza), neratinib maleate (Nerlynx), nilotinib (Tasigna), niraparib tosylate monohydrate (Zejula), nivolumab (Opdivo), obinutuzumab (Gazyva), ofatumumab (Arzerra), olaparib (Lynparza), olaratumab (Lartruvo), osimertinib (Tagrisso), palbociclib (Ibrance), panitumumab (Vectibix), pazopanib (Votrient), pembrolizumab (Keytruda), pemigatinib (Pemazyre), pertuzumab (Perjeta), pexidartinib hydrochloride (Turalio), polatuzumab vedotin-piiq (Polivy), ponatinib hydrochloride (Iclusig), pralatrexate (Folotyn), pralsetinib (Gavreto), radium 223 dichloride (Xofigo), ramucirumab (Cyramza), regorafenib (Stivarga), ribociclib (Kisqali), ripretinib (Qinlock), rituximab (Rituxan), rituximab and hyaluronidase human (Rituxan Hycela), romidepsin (Istodax), rucaparib camsylate (Rubraca), ruxolitinib phosphate (Jakafi), sacituzumab govitecan-hziy (Trodelvy), seliciclib, selinexor (Xpovio), selpercatinib (Retevmo), selumetinib sulfate (Koselugo), siltuximab (Sylvant), sirolimus protein-bound particles (Fyarro), sonidegib (Odomzo), sorafenib (Nexavar), sotorasib (Lumakras), sunitinib (Sutent), tafasitamab-cxix (Monjuvi), tagraxofusp-erzs (Elzonris), talazoparib tosylate (Talzenna), tamoxifen (Nolvadex), tazemetostat hydrobromide (Tazverik), tebentafusp-tebn (Kimmtrak), temsirolimus (Torisel), tepotinib hydrochloride (Tepmetko), tisagenlecleucel (Kymriah), tisotumab vedotin-tftv (Tivdak), tocilizumab (Actemra), tofacitinib (Xeljanz), tositumomab (Bexxar), trametinib (Mekinist), trastuzumab (Herceptin), tretinoin (Vesanoid), tivozanib hydrochloride (Fotivda), toremifene (Fareston), tucatinib (Tukysa), umbralisib tosylate (Ukoniq), vandetanib (Caprelsa), vemurafenib (Zelboraf), venetoclax (Venclexta), vismodegib (Erivedge), vorinostat (Zolinza), zanubrutinib (Brukinsa), ziv-aflibercept (Zaltrap), or any combination thereof.
20. The method of any of clauses 1-19, further comprising obtaining the sample from the subject.
21. The method of any of clauses 1-20, wherein the sample comprises a tissue biopsy sample, a liquid biopsy sample, or a normal control.
22. The method of clause 21, wherein the sample is a liquid biopsy sample and comprises blood, plasma, cerebrospinal fluid, sputum, stool, urine, or saliva.
23. The method of clause 21, wherein the sample is a liquid biopsy sample and comprises circulating tumor cells (CTCs).
24 The method of clause 21, wherein the sample is a liquid biopsy sample and comprises cell-free DNA (cfDNA).
25. The method of clause 24, wherein the cell-free DNA (cfDNA) or a portion thereof comprises circulating tumor DNA (ctDNA).
26. The method of any of clauses 1-25, wherein the plurality of nucleic acid molecules comprises a mixture of tumor nucleic acid molecules and non-tumor nucleic acid molecules.
27. The method of clause 26, wherein the tumor nucleic acid molecules are derived from a tumor portion of a heterogeneous tissue biopsy sample, and the non-tumor nucleic acid molecules are derived from a normal portion of the heterogeneous tissue biopsy sample.
28. The method of clause 26, wherein the sample comprises a liquid biopsy sample, and wherein the tumor nucleic acid molecules are derived from a circulating tumor DNA (ctDNA) fraction of the liquid biopsy sample, and the non-tumor nucleic acid molecules are derived from a non-tumor, cell-free DNA (cfDNA) fraction of the liquid biopsy sample.
29. The method of any of clauses 1-28, wherein the one or more adapters comprise amplification primers, flow cell adaptor sequences, substrate adapter sequences, or sample index sequences.
30. The method of any of clauses 1-29, wherein the captured nucleic acid molecules are captured from the amplified nucleic acid molecules by hybridization to one or more bait molecules.
31 The method of clause 30, wherein the one or more bait molecules comprise one or more nucleic acid molecules, each comprising a region that is complementary to a region of a captured nucleic acid molecule.
32 The method of any of clauses 1-31, wherein amplifying nucleic acid molecules comprises performing a polymerase chain reaction (PCR) amplification technique, a non-PCR amplification technique, or an isothermal amplification technique.
33 The method of any of clauses 1-32, wherein the sequencing comprises use of a massively parallel sequencing (MPS) technique, whole genome sequencing (WGS), whole exome sequencing, targeted sequencing, direct sequencing, or Sanger sequencing technique.
34. The method of clause 33, wherein the sequencing comprises massively parallel sequencing, and the massively parallel sequencing technique comprises next generation sequencing (NGS).
35. The method of any of clauses 1-34, wherein the sequencer comprises a next generation sequencer.
36. The method of any of clauses 1-35, wherein one or more of the plurality of sequencing reads overlap one or more gene loci within one or more subgenomic intervals in the sample.
37 The method of clause 36, wherein the one or more genomic loci comprises between 10 and 20 loci, between 10 and 40 loci, between 10 and 60 loci, between 10 and 80 loci, between 10 and 100 loci, between 10 and 150 loci, between 10 and 200 loci, between 10 and 250 loci, between 10 and 300 loci, between 10 and 350 loci, between 10 and 400 loci, between 10 and 450 loci, between 10 and 500 loci, between 20 and 40 loci, between 20 and 60 loci, between 20 and 80 loci, between 20 and 100 loci, between 20 and 150 loci, between 20 and 200 loci, between 20 and 250 loci, between 20 and 300 loci, between 20 and 350 loci, between 20 and 400 loci, between 20 and 500 loci, between 40 and 60 loci, between 40 and 80 loci, between 40 and 100 loci, between 40 and 150 loci, between 40 and 200 loci, between 40 and 250 loci, between 40 and 300 loci, between 40 and 350 loci, between 40 and 400 loci, between 40 and 500 loci, between 60 and 80 loci, between 60 and 100 loci, between 60 and 150 loci, between 60 and 200 loci, between 60 and 250 loci, between 60 and 300 loci, between 60 and 350 loci, between 60 and 400 loci, between 60 and 500 loci, between 80 and 100 loci, between 80 and 150 loci, between 80 and 200 loci, between 80 and 250 loci, between 80 and 300 loci, between 80 and 350 loci, between 80 and 400 loci, between 80 and 500 loci, between 100 and 150 loci, between 100 and 200 loci, between 100 and 250 loci, between 100 and 300 loci, between 100 and 350 loci, between 100 and 400 loci, between 100 and 500 loci, between 150 and 200 loci, between 150 and 250 loci, between 150 and 300 loci, between 150 and 350 loci, between 150 and 400 loci, between 150 and 500 loci, between 200 and 250 loci, between 200 and 300 loci, between 200 and 350 loci, between 200 and 400 loci, between 200 and 500 loci, between 250 and 300 loci, between 250 and 350 loci, between 250 and 400 loci, between 250 and 500 loci, between 300 and 350 loci, between 300 and 400 loci, between 300 and 500 loci, between 350 and 400 loci, between 350 and 500 loci, or between 400 and 500 loci.
38. The method of clause 36 or 37, wherein the one or more genomic loci comprise ABL1, ACVR1B, AKT1, AKT2, AKT3, ALK, ALOX12B, AMER1, APC, AR, ARAF, ARFRP1, ARID1A, ASXL1, ATM, ATR, ATRX, AURKA, AURKB, AXIN1, AXL, BAP1, BARD1, BCL2, BCL2L1, BCL2L2, BCL6, BCOR, BCORL1, BCR, BRAF, BRCA1, BRCA2, BRD4, BRIP1, BTG1, BTG2, BTK, CALR, CARD11, CASP8, CBFB, CBL, CCND1, CCND2, CCND3, CCNE1, CD22, CD274, CD70, CD74, CD79A, CD79B, CDC73, CDH1, CDK12, CDK4, CDK6, CDK8, CDKN1A, CDKN1B, CDKN2A, CDKN2B, CDKN2C, CEBPA, CHEK1, CHEK2, CIC, CREBBP, CRKL, CSF1R, CSF3R, CTCF, CTNNA1, CTNNB1, CUL3, CUL4A, CXCR4, CYP17A1, DAXX, DDR1, DDR2, DIS3, DNMT3A, DOT1L, EED, EGFR, EMSY (C11orf30), EP300, EPHA3, EPHB1, EPHB4, ERBB2, ERBB3, ERBB4, ERCC4, ERG, ERRFI1, ESR1, ETV4, ETV5, ETV6, EWSR1, EZH2, EZR, FAM46C, FANCA, FANCC, FANCG, FANCL, FAS, FBXW7, FGF10, FGF12, FGF14, FGF19, FGF23, FGF3, FGF4, FGF6, FGFR1, FGFR2, FGFR3, FGFR4, FH, FLCN, FLT1, FLT3, FOXL2, FUBP1, GABRA6, GATA3, GATA4, GATA6, GID4 (C17orf39), GNA11, GNA13, GNAQ, GNAS, GRM3, GSK3B, H3F3A, HDAC1, HGF, HNF1A, HRAS, HSD3B1, ID3, IDH1, IDH2, IGF1R, IKBKE, IKZF1, INPP4B, IRF2, IRF4, IRS2, JAK1, JAK2, JAK3, JUN, KDM5A, KDM5C, KDM6A, KDR, KEAP1, KEL, KIT, KLHL6, KMT2A (MLL), KMT2D (MLL2), KRAS, LTK, LYN, MAF, MAP2K1, MAP2K2, MAP2K4, MAP3K1, MAP3K13, MAPK1, MCL1, MDM2, MDM4, MED12, MEF2B, MEN1, MERTK, MET, MITF, MKNK1, MLH1, MPL, MRE11A, MSH2, MSH3, MSH6, MST1R, MTAP, MTOR, MUTYH, MYB, MYC, MYCL, MYCN, MYD88, NBN, NF1, NF2, NFE2L2, NFKB1A, NKX2-1, NOTCH1, NOTCH2, NOTCH3, NPM1, NRAS, NT5C2, NTRK1, NTRK2, NTRK3, NUTM1, P2RY8, PALB2, PARK2, PARP1, PARP2, PARP3, PAX5, PBRM1, PDCD1, PDCD1LG2, PDGFRA, PDGFRB, PDK1, PIK3C2B, PIK3C2G, PIK3CA, PIK3CB, PIK3R1, PIM1, PMS2, POLD1, POLE, PPARG, PPP2R1A, PPP2R2A, PRDM1, PRKAR1A, PRKC1, PTCH1, PTEN, PTPN11, PTPRO, QKI, RAC1, RAD21, RAD51, RAD51B, RAD51C, RAD51D, RAD52, RAD54L, RAF1, RARA, RB1, RBM10, REL, RET, RICTOR, RNF43, ROS1, RPTOR, RSPO2, SDC4, SDHA, SDHB, SDHC, SDHD, SETD2, SF3B1, SGK1, SLC34A2, SMAD2, SMAD4, SMARCA4, SMARCB1, SMO, SNCAIP, SOCS1, SOX2, SOX9, SPEN, SPOP, SRC, STAG2, STAT3, STK11, SUFU, SYK, TBX3, TEK, TERC, TERT, TET2, TGFBR2, TIPARP, TMPRSS2, TNFAIP3, TNFRSF14, TP53, TSC1, TSC2, TYRO3, U2AF1, VEGFA, VHL, WHSC1, WHSC1L1, WT1, XPO1, XRCC2, ZNF217, ZNF703, or any combination thereof.
39 The method of clause 36 or 37, wherein the one or more genomic loci comprise ABL, ALK, ALL, B4GALNT1, BAFF, BCL2, BRAF, BRCA, BTK, CD19, CD20, CD3, CD30, CD319, CD38, CD52, CDK4, CDK6, CML, CRACC, CS1, CTLA-4, dMMR, EGFR, ERBB1, ERBB2, FGFR1-3, FLT3, GD2, HDAC, HER1, HER2, HR, IDH2, IL-1β, IL-6, IL-6R, JAK1, JAK2, JAK3, KIT, KRAS, MEK, MET, MSI-H, mTOR, PARP, PD-1, PDGFR, PDGFRα, PDGFRB, PD-L1, PI3Kδ, PIGF, PTCH, RAF, RANKL, RET, ROS1, SLAMF7, VEGF, VEGFA, VEGFB, or any combination thereof.
40. The method of any of clauses 1-39, further comprising generating, by the one or more processors, a report indicating the presence or absence of the correlation.
41 The method of clause 40, further comprising transmitting the report to a healthcare provider.
42. The method of clause 41, wherein the report is transmitted via a computer network or a peer-to-peer connection.
43. A method comprising:
   receiving, using one or more processors, genomic data derived from a sample from a subject;
   determining, using the one or more processors, a copy number at one or more genomic loci based on the genomic data;
   determining, using the one or more processors, a distance for each of the one or more genomic loci from a centromere of a chromosome; and
   determining a correlation, using the one or more processors, wherein the correlation comprises a change in the determined copy number as a function of the determined distances.
44. The method of clause 43, further comprising:
detecting, using the one or more processors, a presence or an absence of a disease marker in the sample based on the correlation.
45. The method of clause 44, wherein the disease marker comprises a Howell-Jolly Body (HJB).
46. The method of clause 44 or clause 45, further comprising:
diagnosing a disease in the subject based on the presence or the absence of the disease marker in the sample from the subject.
47. The method of any of clauses 44-46, further comprising:
treating the subject for the disease based on the presence or the absence of the disease marker in the sample from the subject.
48. The method of any of clauses 43-47, wherein the disease comprises asplenia, hyposplenia, or leukopenia.
49. The method of any of clauses 45-48, wherein the presence or the absence of HJBs informs a decision regarding treatment of the subject with an immunotherapy.
50. The method of clause 49, wherein the presence of HJBs informs a contraindication for an immunotherapy.
51. The method of any of clauses 43-50, wherein the correlation is a non-linear correlation.
52 The method of clause 51, wherein the non-linear correlation is an exponential correlation.
53 The method of any of clauses 43-52, wherein the correlation comprises an exponential increase in the copy number as a function of the distance for each of the one or more genomic loci from the centromere of the chromosome.
54 The method of any of clauses 43-50, wherein the correlation is a linear correlation.
55. The method of any of clauses 43-50 or 54, wherein the correlation comprises a linear increase in the copy number as a function of the distance for each of the one or more genomic loci from the centromere of the chromosome.
56 The method of any of clauses 43-55, wherein the correlation is a positive correlation.
57. The method of clause 56, wherein the correlation is positive when the one or more genomic loci are on a q-arm of the chromosome.
58 The method of any of clauses 43-55, wherein the correlation is a negative correlation.
59. The method of clause 58, wherein the correlation is negative when the one or more genomic loci are on a p-arm of the chromosome.
60. The method of any of clauses 43-59, wherein the copy number at the one or more genomic loci comprises a gain or a loss in copy number relative to a baseline copy number.
61 The method of clause 60, wherein the baseline copy number is 2.
62. The method of clause 60 or 61, wherein the gain or the loss in copy number relative to the baseline copy number is a log-normalized gain or loss in the copy number relative to the baseline copy number.
63 The method of clause 62, wherein the log-normalized gain or loss in copy number is log-normalized to base 2.
64. The method of any of clauses 43-63, wherein determining the correlation comprises visualizing the change in the determined copy number as a function of the distance for each of the one or more genomic loci from the centromere of the chromosome in a plot of copy number versus chromosomal position.
65. The method of clause 64, wherein the plot of copy number versus chromosomal position comprises a plot of log-normalized gain or loss in copy number as a function of the distance for each of the one or more genomic loci from the centromere of the chromosome.
66. The method of any of clauses 43-65, wherein determining the correlation comprises fitting a regression model to the change in the determined copy number as a function of the distance for each of the one or more genomic loci from the centromere of the chromosome.
67. The method of clause 66, wherein the fitted regression model comprises a linear regression model.
68. The method of clause 67, wherein the linear regression model comprises a linear regression of a change in log-normalized gain or loss in the copy number as a function of the distance for each of the one or more genomic loci from the centromere of the chromosome.
69. The method of clause 67 or 68, wherein the correlation is indicative of the presence of HJBs in the sample when the linear regression model comprises a slope greater than a predetermined slope or a y-intercept greater than a predetermined y-intercept.
70. The method of clause 66 wherein the fitted regression model comprises a non-linear regression model.
71. The method of clause 70, wherein the non-linear regression model comprises an exponential regression model, a logarithmic regression model, or a logistic regression model.
72. The method of clause 70 or 71, wherein the non-linear regression model comprises a non-linear regression of the change in the gain or loss in the copy number, as the function of the determined distances.
73. The method of any of clauses 43-72, further comprising performing quality control analysis on the received genomic data.
74 The method of clause 73, wherein performing the quality control analysis comprises analyzing a tumor cell fraction for the sample, a genome ploidy for the sample, or a size of a subclonal population of the sample containing copy number variants (CNVs).
75. The method of clause 74, wherein the analyzing the quality control analysis comprises filtering the received genomic data based on the tumor cell fraction for the sample, the genome ploidy for the sample, or the fraction of the subclonal population of the sample containing the CNVs.
76 The method of any of clauses 1-75, wherein the determined distances comprise negative or positive values relative to the centromere.
77. The method of any of clauses 1-76, wherein the determined distances absolute values of the determined distances relative to the centromere.
78. The method of any of clauses 43-77, wherein the received genomic data comprises data for one or more on-target genomic regions and/or one or more off-target genomic regions.
79. The method of any of clauses 43-78, further comprising:
normalizing the received genomic data.
80 The method of clause 79, wherein the normalizing the received genomic data is normalized based on a synthetic normal set of sequence read count data.
81. The method of clause 80, wherein the synthetic normal set of sequence read count data is based on a non-subject profile based on a plurality of non-subject normal samples.
82. The method of any of clauses 79-81, wherein the normalizing the received genomic data comprises normalizing the received genomic data based on the tumor cell fraction for the sample, the genome ploidy for the sample, or the fraction of the subclonal population of the sample containing the CNVs.
83. The method of any of clauses 43-82, wherein the received genomic data comprises sequence read data obtained from next-generation sequencing of the sample.
84. The method of any of clauses 43-83, wherein the received genomic data is obtained from a microarray assay, a PCR-based assay, or a probe displacement-based assay.
85. The method of any of clauses 43-84, further comprising:
   receiving a histopathology image from a blood sample from the subject; and
   detecting a HJB from the received histopathology image.
86. The method of any of clauses 3-85, wherein detecting the HJB in the sample is used to diagnose or confirm a diagnosis of disease in the subject.
87. The method of clause 86, wherein the subject previously underwent a splenectomy, a bone marrow transplant, chemotherapy, or radiation therapy.
88. The method of clause 86 or 87, wherein the disease comprises asplenia, hyposplenia, or leukopenia.
89. The method of clause 88, wherein the disease further comprises sepsis, anemia, immune dysfunction, and/or amyloidosis.
90. The method of any of clauses 86-89, wherein the disease is cancer.
91 The method of clause 90, wherein the cancer is a hematological cancer.
92. The method of any of clauses 1-91, further comprising selecting a therapy to administer to the subject based on the determination of the correlation.
93. The method of clause 92, further comprising determining an effective amount of a therapy to administer to the subject based on the determination of the correlation.
94. The method of clause 93, further comprising administering a therapy to the subject based on the determination of the correlation.
95. The method of any of clauses 92-94, wherein the therapy is an anti-cancer therapy and comprises chemotherapy, radiation therapy, immunotherapy, a targeted therapy, or surgery.
96 The method of any of clauses 92-94, wherein the therapy is a therapy for a disease comprising asplenia, hyposplenia, or leukopenia.
97. A method for diagnosing a disease, the method comprising:
diagnosing that a subject has the disease based on a determination of a correlation between copy number at one or more genomic loci and distance for each of the one or more genomic loci from a centromere of a chromosome for a sample from the subject, wherein the correlation is determined according to the method of any of clauses 1-96.
98. A method of selecting a disease therapy, the method comprising:
responsive to determining a correlation for a sample from a subject, selecting a disease therapy for the subject, wherein the correlation is determined according to the method of any of clauses 1-97.
99. A method of treating a disease in a subject, comprising:
responsive to determining a correlation for a sample from the subject, administering an effective amount of a disease therapy to the subject, wherein the correlation is determined according to the method of any of clauses 1-97.
100. A method for monitoring disease progression or recurrence in a subject, the method comprising:
   determining a first correlation in a first sample obtained from the subject at a first time point according to the method of any of clauses 1-99;
   determining a second correlation in a second sample obtained from the subject at a second time point; and comparing the first correlation to the second correlation, thereby monitoring the disease progression or recurrence.
101. The method of clause 100, wherein the second correlation for the second sample is determined according to the method of any of clauses 1-100.
102. The method of clause 100 or 101, further comprising selecting a therapy for the subject in response to the disease progression.
103. The method of clause 100 or 101, further comprising administering a therapy to the subject in response to the disease progression.
104. The method of clause 100 or 101, further comprising adjusting a disease therapy for the subject in response to the disease progression.
105. The method of any of clauses 102-104, further comprising adjusting a dosage of the disease therapy or selecting a different disease therapy in response to the disease progression.
106. The method of clause 105, further comprising administering the adjusted disease therapy to the subject.
107. The method of any of clauses 100-106, wherein the first time point is before the subject has been administered a disease therapy, and wherein the second time point is after the subject has been administered the disease therapy.
108. The method of any of clauses 100-107, wherein the subject has a disease, is at risk of having a disease, is being routine tested for disease, or is suspected of having a disease.
109. The method of any of clauses 100-108, wherein the disease comprises asplenia, hyposplenia, or leukopenia.
110. The method of clause 109, wherein the disease further comprises sepsis, anemia, immune dysfunction, and/or amyloidosis.
111. The method of clause 109 or 110, wherein the disease is a hematological cancer.
112. The method of any of clauses 100-111 wherein the therapy is a therapy for a disease comprising asplenia, hyposplenia, or leukopenia.
113. The method of any of clauses 100-111, wherein the therapy comprises chemotherapy, radiation therapy, immunotherapy, a targeted therapy, or surgery.
114. The method of any of clauses 1-113, further comprising generating a genomic profile for the subject based on the determination of the correlation.
115. The method of clause 114, wherein the genomic profile for the subject further comprises results from a comprehensive genomic profiling (CGP) test, a gene expression profiling test, a cancer hotspot panel test, a DNA methylation test, a DNA fragmentation test, an RNA fragmentation test, or any combination thereof.
116. The method of clause 114 or 115, wherein the genomic profile for the subject further comprises results from a nucleic acid sequencing-based test.
117. The method of any of clauses 114-116, further comprising selecting a therapy, administering a therapy, or applying a therapy to the subject based on the generated genomic profile.
118. The method of any of clauses 1-117, wherein the determination of the correlation for the sample is used in making suggested treatment decisions for the subject.
119. The method of any of clauses 1-118, wherein the determination of the correlation for the sample is used in applying or administering a treatment to the subject.
120. A system comprising:
   one or more processors; and
   a memory communicatively coupled to the one or more processors and configured to store instructions that, when executed by the one or more processors, cause the system to:
   receive, using the one or more processors, genomic data from a sample from a subject;
   determine, using the one or more processors, a copy number at one or more genomic loci based on the genomic data;
   determine, using the one or more processors, a distance for each of the one or more genomic loci from a centromere of a chromosome; and
   determine a correlation, using the one or more processors, wherein the correlation comprises a change in the determined copy number as a function of the determined distances.
121. The system of clause 120, comprising further instructions that, when executed by the one or more processors, cause the system to:
   detect a presence or an absence of a disease marker in the sample based on the correlation.
122. The system of clause 121, wherein the disease marker comprises a Howell-Jolly Body (HJB).
123. The system of clause 121 or 122, comprising further instructions that, when executed by the one or more processors, cause the system to:
   diagnose a disease in the subject based on the presence or the absence of the disease marker in the sample from the subject.
124. The system of any of clauses 121-123, comprising further instructions that, when executed by the one or more processors, cause the system to:
   treat the subject for the disease based on presence or the absence of the disease marker in the sample from the subject.
125. The system of any of clauses 121-124, comprising further instructions that, when executed by the one or more processors, cause the system to:
   perform quality control analysis on the received genomic data.
126. A non-transitory computer-readable storage medium storing one or more programs, the one or more programs comprising instructions, which when executed by one or more processors of a system, cause the system to:
   receive genomic data from a sample from a subject;
   determine a copy number at one or more genomic loci based on the genomic data;
   determine a distance for each of the one or more genomic loci from a centromere of a chromosome; and
   determine a correlation, wherein the correlation comprises a change in the determined copy number as a function of the determined distances.
127. The non-transitory computer-readable storage medium of clause 126, comprising further instructions that, when executed by the one or more processors, cause the system to:
detect a presence or an absence of a disease marker in the sample based on the correlation.
128. The non-transitory computer-readable storage medium of clause 127, wherein the disease marker comprises a Howell-Jolly Body (HJB).
129. The non-transitory computer-readable storage medium of clause 127 or 128, comprising further instructions that, when executed by the one or more processors, cause the system to:
diagnose a disease in the subject based on the presence of the HJB in the sample from the subject.
130. The non-transitory computer-readable storage medium of any of clauses 127-129, comprising further instructions that, when executed by the one or more processors, cause the system to:
   treat the subject for the disease based on the presence or the absence of the disease marker in the sample from the subject.
131. The non-transitory computer-readable storage medium of any of clauses 127-130, comprising further instructions that, when executed by the one or more processors, cause the system to:
   perform quality control analysis on the received genomic data.

It should be understood from the foregoing that, while particular implementations of the disclosed methods and systems have been illustrated and described, various modifications can be made thereto and are contemplated herein. It is also not intended that the invention be limited by the specific examples provided within the specification. While the invention has been described with reference to the aforementioned specification, the descriptions and illustrations of the preferable embodiments herein are not meant to be construed in a limiting sense. Furthermore, it shall be understood that all aspects of the invention are not limited to the specific depictions, configurations or relative proportions set forth herein which depend upon a variety of conditions and variables. Various modifications in form and detail of the embodiments of the invention will be apparent to a person skilled in the art. It is therefore contemplated that the invention shall also cover any such modifications, variations and equivalents.

### EXAMPLES

The following examples further demonstrate to one skilled in the art how to make and use the methods and systems described herein and are not intended to limit the scope of the claimed invention.

### Example 1—Detecting Characteristic Genomic Signatures of Howell-Jolly Bodies

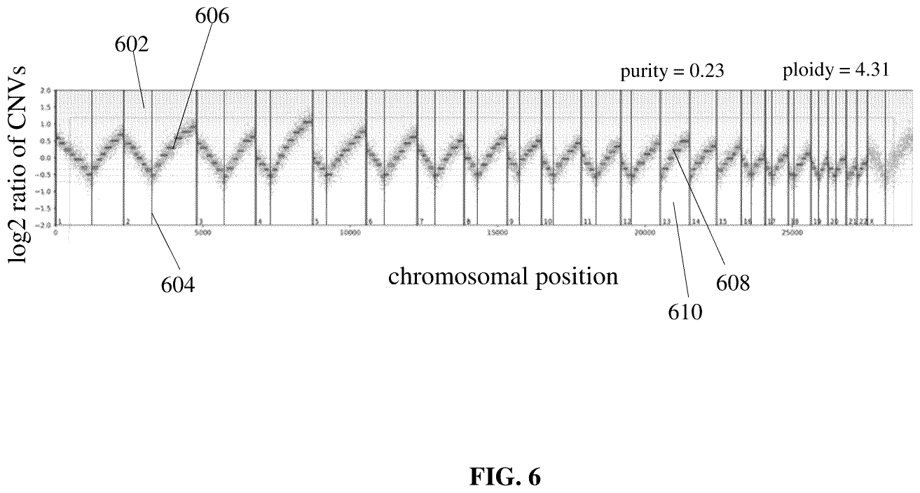

A single blood sample was obtained from one subject with myelodysplastic syndrome. The sample was sequenced and libraries were prepared according to standard protocols. The sample was assessed for its tumor cell fraction, e.g., purity, and its ploidy. The sample was determined to possess a purity of 0.23 and a ploidy of 4.31. FIG. 6 depicts a plot visualizing the log 2 ratio of normalized copy number variant (CNV) data as a function of chromosomal position, for each chromosome, where the CNV data was derived from genomic (e.g., sequencing) data. Each box within the plot, such as box 602, depicts the log 2 ratio of normalized CNV data for a particular chromosome. For example, box 602 depicts the log 2 ratio of normalized CNV data for chromosome 2. For each box within the plot, such as box 602, a vertical line depicts the position of the centromere for the particular chromosome. For example, for box 602, vertical line 604 depicts the position of the centromere for chromosome 2. Data 606 for chromosome 2 depicts a “V-shaped” trend where the plot of the log 2 ratio of the normalized CNV data resembles a “V” centered on the position of the centromere. That is, from the chromosome's distal end on the p-arm to approximately the centromere, a downward trend in the data 606 can be observed, and from approximately the centromere to the chromosome's distal end on the q-arm of the centromere, an upward trend in the data 606 can be observed. This “V-shaped” trend (e.g., the genomic signature indicating a presence of a Howell-Jolly Body) has only been observed in patient samples for which the presence of Howell-Jolly Bodies (HJBs) was confirmed in pathology images. In this example, the “V-shaped” trend arises due to a linear correlation between the log 2 ratio of normalized CNV data and chromosomal position or the chromosomal distance from the centromere. In some instances, the correlation between the log 2 ratio of normalized CNV data and chromosomal position or the chromosomal distance from the centromere can be a non-linear, e.g., exponential, correlation.

In contrast to box 604 which depicts data for chromosome 2, a metacentric chromosome, some of the boxes in FIG. 6 depict data for acrocentric chromosomes. For example, box 610 depicts the log 2 ratio of normalized CNV data for chromosome 13, which is an acrocentric chromosome. For data related to acrocentric chromosomes, such as the data depicted in box 610, a vertical line depicting the position of the centromere is not readily visible because acrocentric chromosomes possess centromeres located at or near one of the distal ends of the chromosome. Although the data 608 plotted in box 610 for chromosome 13 does not depict a ‘V-shaped’ trend (because the chromosome is acrocentric), the data 608 still shows a trend that is in accordance with a linear correlation between the log 2 ratio of the normalized CNV data and chromosomal position or chromosomal distance from the centromere. Again, the trend observed in data 608 (e.g., an alternative genomic signature indicating a presence of a HJB) is indicative of the presence of HJBs in the sample. In some instances, the correlation between the log 2 ratio of normalized CNV data and chromosomal position or the chromosomal distance from the centromere can be a non-linear, e.g., exponential, correlation.

It should be understood from the foregoing that, while particular implementations of the disclosed methods and systems have been illustrated and described, various modifications can be made hereto and are contemplated herein. It is also not intended that the invention be limited by the specific examples provided within the specification. While the invention has been described with reference to the aforementioned specification, the descriptions and illustrations of the preferable embodiments herein are not meant to be construed in a limiting sense. Furthermore, it shall be understood that all aspects of the invention are not limited to the specific depictions, configurations, or relative proportions set forth herein which depend upon a variety of conditions and variables. Various modifications in form and detail of the embodiments of the invention will be apparent to a person skilled in the art. It is therefore contemplated that the invention shall also cover any such modifications, variations, and equivalents.

1-42. (canceled)

43. A method of detecting a Howell-Jolly Body (HJB) in a subject, comprising:
receiving, at one or more processors, genomic data derived from a sample from the subject;
determining, using the one or more processors, a copy number at one or more genomic loci based on the genomic data;
determining, using the one or more processors, a distance for each of the one or more genomic loci from a centromere of a chromosome; and
detecting, using the one or more processors, the HJB in the subject based on a change in the determined copy number as a function of the determined distances.

44-45. (canceled)

46. The method of claim 43, further comprising: diagnosing a disease in the subject based on the detection of the HJB.

47. The method of claim 46, further comprising: treating the subject for the disease based on the detection of the HJB.

48. The method of claim 46, wherein the disease comprises asplenia, hyposplenia, leukopenia, sepsis, anemia, immune dysfunction, and/or amyloidosis.

49. The method of claim 46, wherein the detection of the HJB informs a decision regarding treatment of the subject with an immunotherapy.

50. The method of claim 49, wherein the detection of the HJB informs a contraindication for an immunotherapy.

51-61. (canceled)

62. The method of claim 43, wherein the change in the determined copy number is detected using a log-normalized count of sequencing reads.

63. The method of claim 62, wherein the log-normalized count of sequencing reads is log-normalized to base 2.

64. The method of claim 43, wherein detecting the HJB comprises visualizing the change in the determined copy number as a function of the distance for each of the one or more genomic loci from the centromere of the chromosome in a plot of copy number versus chromosomal position.

65. The method of claim 64, wherein the plot of copy number versus chromosomal position comprises a plot of a log-normalized count of sequencing reads as a function of the distance for each of the one or more genomic loci from the centromere of the chromosome.

66. The method of claim 43, wherein detecting the HJB comprises fitting a regression model to the change in the determined copy number as a function of the distance for each of the one or more genomic loci from the centromere of the chromosome.

67. The method of claim 66, wherein the fitted regression model comprises a linear regression model.

68. The method of claim 67, wherein the linear regression model comprises a linear regression of the change in copy number as a function of the distance for each of the one or more genomic loci from the centromere of the chromosome.

69. The method of claim 67, wherein the HJB is detected when the linear regression model comprises a slope greater than a predetermined slope or a y-intercept greater than a predetermined y-intercept.

70. The method of claim 66, wherein the fitted regression model comprises a non-linear regression model.

71. The method of claim 70, wherein the non-linear regression model comprises an exponential regression model, a logarithmic regression model, or a logistic regression model.

72. The method of claim 70, wherein the non-linear regression model comprises a non-linear regression of the change in copy number, as the function of the determined distances.

73-84. (canceled)

85. The method of claim 43, further comprising:
receiving a histopathology image from a blood sample from the subject; and
detecting the HJB from the received histopathology image.

86-98. (canceled)

99. A method of treating a disease in a subject, comprising: responsive to detecting the HJB in the subject, administering an effective amount of a disease therapy to the subject, wherein the HJB is detected according to the method of claim 43.

100-119. (canceled)

120. A system for detecting a Howell-Jolly Body (HJB) in a subject, comprising:
one or more processors; and
a memory communicatively coupled to the one or more processors and configured to store instructions that, when executed by the one or more processors, cause the system to:
receive, at the one or more processors, genomic data from a sample from the subject;
determine, using the one or more processors, a copy number at one or more genomic loci based on the genomic data;
determine, using the one or more processors, a distance for each of the one or more genomic loci from a centromere of a chromosome; and
detecting, using the one or more processors, the HJB in the subject based on a change in the determined copy number as a function of the determined distances.

121-131. (canceled)
